SEQUENCE LISTING (132 sequences)

SEQ ID NO: 1 - DNA, 2578 bp, Artificial sequence
  Tol1 transposase plasmid clone sequence
tatcgatgccaccatggaaaagaagcggagcaagccttctggcgctcaatttagaaagaaaagaaaggaagaggaagaaaagagagataaggaaaagggcgccctgctgagatactttggcagctccaccaccgcccaggacgagaccagcaccagcctgccagctatctctagcgcaaccgtgaccgtgtcccctccacaggatgagctgcctagcacaagctctgccacccacgtggtgcctcagcttctgcctgagcagtctttcgatagcgaggccgaagatgtggtcccctctacgtccacccagctggaaaccagcgagatgcctggcgacgagacacctctgacccctaccgccgaggaccagcctctgcctactgatcctgccaagtggcctagccccctgaccgatagaatcagaatggaactggttagaagaggaccttctagtatcccccccgacttcgtgttccccagaaacgacagcgatggcagatcatgccaccatcactacttccggaagaccctggtgagcggcgagaagatcgccagaacctggctgatgtacagcaaggtgaagaatagcctgttttgcttctgttgtaagctgttctctaacaagaatatcaacctcaccacctctggaacagcgaactggaagcacgccagcacttacctgaccgcccacgagaagagccccgagcacctgaactgcatgaaagcctggaaagaactgtccggccggattcggagcggaaaaaccatcgacaagcaggagatggccctgctggaagaggaacgggtgcgctggcgggccgtgctcacacggctgatcgccatcgtgcagtctctggctgtgcgcaacctggccctgagaggccacacagagacactgttcaccagcagcaacggcaacttcctgaaggaagtggagctgatggctagattcgaccctatcatgaaggaccacctgaacagagtgctgagaggcaccgcctcccataattcttatatcggacaccacgtacaaaacgagctgatcgacctgctgagcagcaaaatcctgtctgccatcgttgacgacatcaagaaggccaagtactttagcatcatcctggattgcaccctggacatcagccacaccgagcagctgagcgtgatcatcagagtcgtgagcctgatggaaaagccccagataagagagcacttcatgggcttcctggaggccgaggaaagtacaggccagcacctggcctctatgatcctgaacagactggaagaactgggcatcagcttcgaggactgtcggggccagagctatgacaacggcgctaatatgaaaggcaagaacaagggcgtccaggccagactgctggagaagaacccccgggccctgtttctgccctgtggcgctcataccctgaatctggtggtgtgcgatgcagctaagagatccgtggacgccatgagctacttcggcgtgctccagaaactgtacaccctgttctctgcgagcgcccagcggtgggccatcctgaagagccaggtgtctattacactgaagagctggaccgaaaccagatgggagtctaagatcaagagcatcgagcctatgcggtaccagggagccgccgtgagggaagctctcatcgaggtgcgggacaaaaccaaggaccccgtgatcaaagctgaggcccagagcctgtctgaggaagttggaagctacagattcaacatctgcacagtggtgtggcacgacatcctatccacaatcaagcacgtgagtaagctgatgcagtctcctaacatgcacgtggatctggccgtgagcctgctaaaaaaaaccgagcagagccttcagtcttacagagccaatggattcgtgaacgcccaaatggccgccaaggaaatgtgcaaggaaatgaacgtggaagctatcctgaaacagaagcggattaggagcacaaagtgccagttctcgtacgagagccacgatgagcccttcagcgacgccctcaagaagctggaagtggagttcttcaacgtggtggtggacgaggctctgtccgccatcgctgagcgcttcagcacactggaagtggtccagaacagattcggcgtgctgaccaacttcccttccctgggcgatgaggagcttacagagcagtgcgaggccctgggcaacattctgcacttcgagaaaaattgggacctggacagcagagagctggtgcaggagatcaagaacctgccgaacttaccgagcaccacaccttccctgctggaactgatcagcttcatgtccgacaaggacctgtcagaaatttaccctaacttttggaccgccctgagaatcgccctgacactccctgtgaccgtggcccaggccgagagaagcttctccaagctgaagctgatcaagtcatacctgagaagcaccatgtctcaggagagactgacaaacctggccgtggtgtctatcaaccacagcgtgggcgaacaaatcagctacgatgatgtgatcgacgagttcgccagcagaaaagccagaaaagtgcggttttgataactcgag

SEQ ID NO: 2 - protein, 851 aa, Artificial sequence
  Tol1 transposase amino acid sequence
MEKKRSKPSGAQFRKKRKEEEEKRDKEKGALLRYFGSSTTAQDETSTSLPAISSATVTVSPPQDELPSTSSATHVVPQLLPEQSFDSEAEDVVPSTSTQLETSEMPGDETPLTPTAEDQPLPTDPAKWPSPLTDRIRMELVRRGPSSIPPDFVFPRNDSDGRSCHHHYFRKTLVSGEKIARTWLMYSKVKNSLFCFCCKLFSNKNINLTTSGTANWKHASTYLTAHEKSPEHLNCMKAWKELSGRIRSGKTIDKQEMALLEEERVRWRAVLTRLIAIVQSLAVRNLALRGHTETLFTSSNGNFLKEVELMARFDPIMKDHLNRVLRGTASHNSYIGHHVQNELIDLLSSKILSAIVDDIKKAKYFSIILDCTLDISHTEQLSVIIRVVSLMEKPQIREHFMGFLEAEESTGQHLASMILNRLEELGISFEDCRGQSYDNGANMKGKNKGVQARLLEKNPRALFLPCGAHTLNLVVCDAAKRSVDAMSYFGVLQKLYTLFSASAQRWAILKSQVSITLKSWTETRWESKIKSIEPMRYQGAAVREALIEVRDKTKDPVIKAEAQSLSEEVGSYRFNICTVVWHDILSTIKHVSKLMQSPNMHVDLAVSLLKKTEQSLQSYRANGFVNAQMAAKEMCKEMNVEAILKQKRIRSTKCQFSYESHDEPFSDALKKLEVEFFNVVVDEALSAIAERFSTLEVVQNRFGVLTNFPSLGDEELTEQCEALGNILHFEKNWDLDSRELVQEIKNLPNLPSTTPSLLELISFMSDKDLSEIYPNFWTALRIALTLPVTVAQAERSFSKLKLIKSYLRSTMSQERLTNLAVVSINHSVGEQISYDDVIDEFASRKARKVRF

SEQ ID NO: 3 - DNA, 200 bp, Artificial sequence
  Tol1 transposon 5′ terminal inverted repeat
cagtagcggttctaggcacgggccgtccgggcggtggcctggggcggaaaactgaaggggggcggcaccggcggctcagccctttgtaatatattaatatgcaccactattggtttacttatgtcacagtttgtaagtttgtaacagcctgaacctggccgcgccgccgccctcgccccgcagctgcgctctcctgtctt

SEQ ID NO: 4 - DNA, 505 bp, Artificial sequence
  Tol1 transposon 3′ terminal inverted repeat
atatttttagccaatagaatttccataaatctgtaggtagttttaaaaatgaatatttaccatttactgcaactctatggggacaaaacataatgtaacaggtcataactaaaaatgtgccaatcaaaggattgaagacggaaaacatgagttaatttttcttctctgaagtagagttcgatatagaacatgacaatttaaatttccaattcataaatgtttttaaaatatttattttatattatttatttaacattgagtttgattcaatattttcttagctaactgtatttttgccatgcttatggtcttttattttttgtgttctgataacttttataatgcttttcagaattttgacatcttttgtatccacttcttaatttcaatgacaataaaacatttcagttgacgaagacaaacaaagttctgttgtgactatggggggggggggcgcctggggatggtctcgcccggggagtaattcagggtagaaccgccactg

SEQ ID NO: 5 - DNA, 764 bp, Artificial sequence
  Tol1 transposon plasmid clone sequence
gcggccgccctttagccagtagcggttctaggcacgggccgtccgggcggtggcctggggcggaaaactgaaggggggcggcaccggcggctcagccctttgtaatatattaatatgcaccactattggtttacttatgtcacagtttgtaagtttgtaacagcctgaacctggccgcgccgccgccctcgccccgcagctgcgctctcctgtcttcctgcaggtacgatcaagcggcgcgccatatttttagccaatagaatttccataaatctgtaggtagttttaaaaatgaatatttaccatttactgcaactctatggggacaaaacataatgtaacaggtcataactaaaaatgtgccaatcaaaggattgaagacggaaaacatgagttaatttttcttctctgaagtagagttcgatatagaacatgacaatttaaatttccaattcataaatgtttttaaaatatttattttatattatttatttaacattgagtttgattcaatattttcttagctaactgtatttttgccatgcttatggtcttttattttttgtgttctgataacttttataatgcttttcagaattttgacatcttttgtatccacttcttaatttcaatgacaataaaacatttcagttgacgaagacaaacaaagttctgttgtgactatggggggggggggcgcctggggatggtctcgcccggggagtaattcagggtagaaccgccactgcctttagcgcgatcgc

SEQ ID NO: 6 - DNA, 1972 bp, Artificial sequence
  Tol2 transposase plasmid clone sequence
tatcgatgccaccatggaagaggtgtgcgacagctctgctgccgcctcttcaacagtgcagaaccagcctcaggaccaggagcacccctggccttacctgcgggagttcttcagtctgagcggagtgaacaaggacagcttcaagatgaagtgcgtgctgtgcctgccactgaacaaggaaattagcgccttcaagtcttctccaagcaacctgcggaagcacatcgagagaatgcaccctaactacctgaagaactacagcaaattgacagctcagaagcggaagatcggcacctccacacacgccagcagcagcaagcagctgaaagtcgatagcgtgtttcctgtgaagcacgtgtcgcccgtgaccgtgaataaggccattctgagatacatcatccagggcctgcatccattcagcaccgtggacctgccttccttcaaagaactgatctctaccctgcagcccggcatcagcgtgatcacccggcctaccctgagatccaagatcgcggaggccgctctgatcatgaagcaaaaggtgaccgccgctatgagcgaagtggaatggatcgccaccacaaccgactgctggacggccagaagaaagtccttcatcggcgtgaccgcccattggattaaccctggctcactggagagacacagcgccgccctggcctgcaagcggctgatgggctctcataccttcgaagtcctggcctccgccatgaacgacatccacagcgagtacgagatccgggacaaagttgtgtgtaccaccaccgacagcggcagcaatttcatgaaagcctttagagtattcggcgtggagaacaatgacatcgagacagaggcccggaggtgcgagagcgacgacacagatagtgaaggatgtggcgaaggctccgacggcgtggaatttcaggatgcctctagggtcctggatcaggacgacggatttgagttccagctgcccaagcaccaaaaatgcgcctgtcacctgctgaacctggtgagctctgtggatgcccagaaggccctgagcaacgagcactacaagaagctttatagaagcgtgttcggaaagtgtcaggctctgtggaacaaaagcagcagaagcgccctggccgctgaggccgtggagagcgagagccggctgcagctgctgcggcctaaccagacaagatggaatagcaccttcatggccgtggatagaatcctccaaatctgcaaggaggccggcgagggcgccctgcgcaacatctgcacctctctggaggtgcctatgttcaaccccgctgaaatgctgttcctgaccgagtgggccaacaccatgagacccgtggccaaggtgctggacatcctgcaggctgagacaaacacccagctgggctggttgctgcctagcgtgcaccagctgagcctgaaactgcaaagactgcaccactctctgagatactgcgaccccctggtggacgccctgcagcagggtatccagaccagattcaagcacatgtttgaagaccctgagatcatcgccgctgccatcctgctgcctaagttccggacaagctggacaaatgatgaaaccatcatcaagagaggcatggactacatcagagttcacctggaacctctggatcacaagaaagagctggcaaacagcagtagcgatgacgaggacttcttcgccagcctcaagcctacaacccacgaggccagcaaagaactggacggctacctggcctgcgtgtccgacaccagagagagcctgctgaccttccccgctatctgtagcctgagcatcaagacaaatacccctctgcctgccagcgccgcctgcgagagactgttctccaccgctggcctgcttttcagccctaagagagccagactggatacaaacaactttgagaaccagctcctgctgaaactcaatctgcggttctacaacttcgaatgataactcgag

SEQ ID NO: 7 - protein, 649 aa, Artificial sequence
  Tol2 transposase amino acid sequence
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 8 - DNA, 200 bp, Artificial sequence
  Tol2 transposon 5′ terminal inverted repeat
cagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcactt

SEQ ID NO: 9 - DNA, 150 bp, Artificial sequence
  Tol2 transposon 3′ terminal inverted repeat
aatactcaagtacaattttaatggagtacttttttacttttactcaagtaagattctagccagatacttttacttttaattgagtaaaattttccctaagtacttgtactttcacttgagtaaaatttttgagtactttttacacctctg

SEQ ID NO: 10 - DNA, 459 bp, Artificial sequence
  Tol2 transposon plasmid clone sequence
gcggccgcggtaatcccagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcctgcaggtacgatcaagcggcgcgcccttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctgggtaatccgcgatcgc

SEQ ID NO: 11 - DNA, 1045 bp, Artificial sequence
  Frog prince transposase plasmid clone sequence
aatcgatgccaccatgcctcggcccaaggaaatccaagaacagctgcggaagaaagtgattgagatttaccagtctggcaagggctacaaggctatcagcaaggccctgggcatccagcggaccacagtgcgggccatcatccacaagtggagacggcacggcaccgtggtgaacctgcctagaagcggcagacctccaaaaatcacccctagagcccagagaagactgatccaggaggtgaccaaagatcctaccaccacaagcaaggagctgcaggccagcctcgcctctgtgaaggtgtccgtgcacgccagcaccatcagaaagcggcttggaaagaacggcctgcacggccgcgtgcccagacggaagcctctgctgagcaagaagaatatcaaggctagactgaacttcagcaccacccacctggatgaccctcaggacttctgggacaacatcctgtggaccgacgagacaaaggtggaactgttcggccggtgcgtgtctaaatacatctggagacggagaaacaccgccttccataagaagaatatcatccccacagtcaagtacggcggcggaagcgtgatggtgtggggctgcttcgccgccagcggccctggcagactggccgtgatcaagggcacaatgaacagcgccgtgtaccaagagatcctgaaggaaaacgtgaggccttctgtcagggtgctgaagctgaagagaacctgggtgctgcagcaggataacgaccccaagcacaccagcaagtcaacaacagagtggctgaagaagaacaagatgaagaccctggaatggcccagccagagccctgatctgaaccctatcgagatgctgtggtacgacctgaaaaaggccgtgcacgccagaaaaccctccaatgtcaccgagctgggccagttttgtaaagacgagtgggccaagatcccaccaggcagatgcaagtccctgatcgccagatatagaaaaagactggtggccgttgtggctgctaagggaggacctactagctactgataactcgag

SEQ ID NO: 12 - protein, 340 aa, Artificial sequence
  Frog prince transposase amino acid sequence
MPRPKEIQEQLRKKVIEIYQSGKGYKAISKALGIQRTTVRAIIHKWRRHGTVVNLPRSGRPPKITPRAQRRLIQEVTKDPTTTSKELQASLASVKVSVHASTIRKRLGKNGLHGRVPRRKPLLSKKNIKARLNFSTTHLDDPQDFWDNILWTDETKVELFGRCVSKYIWRRRNTAFHKKNIIPTVKYGGGSVMVWGCFAASGPGRLAVIKGTMNSAVYQEILKENVRPSVRVLKLKRTWVLQQDNDPKHTSKSTTEWLKKNKMKTLEWPSQSPDLNPIEMLWYDLKKAVHARKPSNVTELGQFCKDEWAKIPPGRCKSLIARYRKRLVAVVAAKGGPTSY

SEQ ID NO: 13 - DNA, 250 bp, Artificial sequence
  Frog prince transposon terminal inverted repeat
cagtggtgtgaaaaagtgtttgcccccttcctcatttcctgttcctttgcatgtttgtcacacttaagtgtttcggaacatcaaaccaatttaaacaatagtcaaggacaacacaagtaaacacaaaatgcaatttgtaaatgaaggtgtttattattaaaggtgaaaaaaaatccaaaccatcatggccctgtgtgaaaaagtgattgccccccttgttaaaacatactataactgtggttgtccacac

SEQ ID NO: 14 - DNA, 551 bp, Artificial sequence
  Frog prince transposon plasmid clone sequence
gcggccgctatacagtggtgtgaaaaagtgtttgcccccttcctcatttcctgttcctttgcatgtttgtcacacttaagtgtttcggaacatcaaaccaatttaaacaatagtcaaggacaacacaagtaaacacaaaatgcaatttgtaaatgaaggtgtttattattaaaggtgaaaaaaaatccaaaccatcatggccctgtgtgaaaaagtgattgccccccttgttaaaacatactataactgtggttgtccacaccctgcaggtacgatcaagcggcgcgccgtgtggacaaccacagttatagtatgttttaacaaggggggcaatcactttttcacacagggccatgatggtttggattttttttcacctttaataataaacaccttcatttacaaattgcattttgtgtttacttgtgttgtccttgactattgtttaaattggtttgatgttccgaaacacttaagtgtgacaaacatgcaaaggaacaggaaatgaggaagggggcaaacactttttcacaccactgtatagcgatcgc

SEQ ID NO: 15 - DNA, 1048 bp, Artificial sequence
  Minos transposase plasmid clone sequence
tatcgatgccaccatggtgcgcggaaagcctatcagcaaggaaatccgggtgctgatcagagattactttaagtccggcaaaaccctgaccgagatcagcaagcagctgaatctgcccaagagctctgtgcacggcgtgattcaaatcttcaagaagaacggcaacattgaaaacaacatcgccaacagaggcagaaccagcgctatcacacctagagacaagcggcagctcgccaagatcgtgaaagccgatagaagacagagcctgcggaacctggcctccaagtggtcccagaccatcggaaagaccgttaagagagagtggacaagacagcagctgaaaagcatcggctacggcttctacaaggccaaggaaaagcctctgctgaccctgagacaaaagaagaaacggctgcagtgggccagagaaagaatgagctggacccagcggcagtgggacaccatcatcttcagcgacgaggccaaattcgacgtgtccgtgggcgacacaagaaagcgcgtgatcagaaagcggagcgagacatatcacaaggactgcctgaagaggaccacaaaattccccgccagcaccatggtctggggctgcatgagcgccaaaggtctgggaaagctgcatttcatcgagggcaccgtgaacgccgaaaagtacatcaacatcctgcaggacagcctgctgccttctatccccaagctgtctgattgcggcgagttcaccttccagcaagacggcgcttcttctcacaccgccaagcggacaaagaattggctgcaatacaaccagatggaagtgctggactggcccagcaacagccctgatctgagcccaatcgagaacatctggtggctgatgaaaaaccagctcagaaacgagcctcagagaaatattagcgacctgaagatcaagctgcaggagatgtgggatagcatctctcaggagcactgtaaaaatctgctgagcagcatgcctaagcgggtgaagtgcgtgatgcaagctaagggcgacgtgacacagttctgataactcgag

SEQ ID NO: 16 - protein, 341 aa, Artificial sequence
  Minos transposase amino acid sequence
MVRGKPISKEIRVLIRDYFKSGKTLTEISKQLNLPKSSVHGVIQIFKKNGNIENNIANRGRTSAITPRDKRQLAKIVKADRRQSLRNLASKWSQTIGKTVKREWTRQQLKSIGYGFYKAKEKPLLTLRQKKKRLQWARERMSWTQRQWDTIIFSDEAKFDVSVGDTRKRVIRKRSETYHKDCLKRTTKFPASTMVWGCMSAKGLGKLHFIEGTVNAEKYINILQDSLLPSIPKLSDCGEFTFQQDGASSHTAKRTKNWLQYNQMEVLDWPSNSPDLSPIENIWWLMKNQLRNEPQRNISDLKIKLQEMWDSISQEHCKNLLSSMPKRVKCVMQAKGDVTQF

SEQ ID NO: 17 - DNA, 257 bp, Artificial sequence
  Minos transposon terminal inverted repeat
atacgagccccaaccactattaattcgaacagcatgttttttttgcagtgcgcaatgtttaacacactatattatcaatactactaaagataacacataccaatgcatttcgtctcaaagagaattttattctcttcacgacgaaaaaaaaagttttgctctatttccaacaacaacaaaaatatgagtaatttattcaaacggtttgcttaagagataagaaaaaagtgaccactattaattcgaacgcggcgtaa

SEQ ID NO: 18 - DNA, 561 bp, Artificial sequence
  Minos transposon plasmid clone sequence
gcggccgctaatacgagccccaaccactattaattcgaacagcatgttttttttgcagtgcgcaatgtttaacacactatattatcaatactactaaagataacacataccaatgcatttcgtctcaaagagaattttattctcttcacgacgaaaaaaaaagttttgctctatttccaacaacaacaaaaatatgagtaatttattcaaacggtttgcttaagagataagaaaaaagtgaccactattaattcgaacgcggcgtaacctgcaggtacgatcaagcggcgcgccttacgccgcgttcgaattaatagtggtcacttttttcttatctcttaagcaaaccgtttgaataaattactcatatttttgttgttgttggaaatagagcaaaactttttttttcgtcgtgaagagaataaaattctctttgagacgaaatgcattggtatgtgttatctttagtagtattgataatatagtgtgttaaacattgcgcactgcaaaaaaaacatgctgttcgaattaatagtggttggggctcgtattagcgatcgc

SEQ ID NO: 19 - DNA, 1054 bp, Artificial sequence
  Hsmar1 transposase plasmid clone sequence
aatcgatgccaccatggaaatgatgctggacaagaagcagatccgggccatcttcctgttcgagttcaagatgggcagaaaggccgccgaaaccaccaggaacatcaacaacgcctttggccctggaacagccaatgagcggaccgtgcagtggtggttcaaaaagttccggaagggcgacgagagcctggaagatgaggaacggagcggcagacccagcgaagtggacaacgaccagctgagagccattatcgaggctgatcctctgaccaccactagagaggttgccgaggaactgaacgtggaccactctacagtggtccggcacctgaagcagatcggcaaggtgaagaaactggacaagtgggtcccccacgagctgagcgagaaccagaaaaacagaagattcgaggtgtccagcagcctgctgctgagaaacaacaatgagcctttcctggatagaatcgtgacctgtgacgaaaagtggatcctgtacgacaacagacgccggagcgcccagtggctggatagagaagaagcccctaagcactttccaaagccaaacctgcaccagaagaaagtgatggtgacagtgtggtggtccgccgctggcgtgatccattattcttttctgaatcctggagaaaccatcaccagcgagaagtactgccagcagatcgacgagatgcacagaaagctccaaagactgcagcctgccctggtgaatcggaagggccccatcctgctgcacgacaacgccagacctcatgtggctcaacctacactgcaaaaactgaatgagctgggctacgaggtgctgcctcaccccccctacagccctgacctgtctcctaccgattaccacttcttcaagcacctcgataacttcctgcagggcaagcggttccacaaccagcaggacgccgagaacgcttttcaggagttcgtggaaagcagaagcacagatttctacgccaccggaatcaacaagctgatctctagatggcagaaatgcgtggactgcaacggcagctacttcgactgataactcgag

SEQ ID NO: 20 - protein, 343 aa, Artificial sequence
  Hsmar1 transposase amino acid sequence
MEMMLDKKQIRAIFLFEFKMGRKAAETTRNINNAFGPGTANERTVQWWFKKFRKGDESLEDEERSGRPSEVDNDQLRAIIEADPLTTTREVAEELNVDHSTVVRHLKQIGKVKKLDKWVPHELSENQKNRRFEVSSSLLLRNNNEPFLDRIVTCDEKWILYDNRRRSAQWLDREEAPKHFPKPNLHQKKVMVTVWWSAAGVIHYSFLNPGETITSEKYCQQIDEMHRKLQRLQPALVNRKGPILLHDNARPHVAQPTLQKLNELGYEVLPHPPYSPDLSPTDYHFFKHLDNFLQGKRFHNQQDAENAFQEFVESRSTDFYATGINKLISRWQKCVDCNGSYFD

SEQ ID NO: 21 - DNA, 30 bp, Artificial sequence
  Hsmar1 transposon terminal inverted repeat
ttaggttggtgcaaaagtaattgcggtttt

SEQ ID NO: 22 - DNA, 302 bp, Artificial sequence
  Hsmar1 transposon plasmid clone sequence
gcggccgctattaggttggtgcaaaagtaattgcggtttttgcattgttggaatttgccgtttgatattggaatacattcttaaataaatgtggttatgttatacatcattttaatgcgcatttctcgctttacgtttttttgctaatgacttattacttgctgtttattttatgtttattttagactcctgcaggtacgatcaagcggcgcgcctaaagatgtgtttgagcctagttataatgatttaaaattcacggtccaaaaccgcaattacttttgcaccaacctaatagcgatcgc

SEQ ID NO: 23 - DNA, 4514 bp, Artificial sequence
  Helraiser transposase plasmid clone sequence
aatcgatgccaccatgagcaaggaacagctcctaatccagagaagctctgccgccgaaagatgcagaagatacagacaaaagatgagcgcagagcaaagagcctctgacctggaaagaagacggcggctgcagcaaaacgtgagcgaggaacaactgctggagaagcggcgaagcgaggccgagaagcagcggcggcaccggcagaagatgagcaaagaccagagagccttcgaggtggaaaggagaaggtggcggagacaaaatatgtctcgggaacagagcagcacaagcacgaccaacaccggcagaaactgcctgctgagcaagaacggcgtgcatgaggatgctatcctggaacacagctgcggcggcatgaccgtgcggtgcgagttttgcctgtctctgaatttcagcgacgagaagccaagcgatggaaagttcaccagatgctgcagcaaaggcaaggtgtgccccaatgacatacacttcccagactacccggcctatctgaagcggctgatgaccaatgaggactcagattctaagaatttcatggaaaacatcagaagcattaactccagcttcgcttttgccagcatgggcgccaatatcgccagcccttctggctacggcccttattgcttcagaatccacggccaggtctaccacagaacaggcactttgcaccctagcgatggagtgtcccggaagtttgcccagctgtacatcctggatacagccgaggctacaagcaagcggctggccatgcccgagaaccagggctgtagcgagcgcctgatgatcaacatcaataacctgatgcacgagatcaacgagctgacaaagagctacaagatgctgcacgaggtcgagaaagaggctcagagcgaagccgctgccaagggaattgcccctactgaggtgacaatggccatcaagtacgacagaaactctgaccctggccggtataacagccctcgggtgaccgaagtggctgtgatatttaggaacgaggacggcgaaccccccttcgagagagacctgctgatccactgcaagcccgaccctaacaatcctaatgccaccaagatgaagcagatcagcatcctgttccctaccctggacgcaatsgacctaccctatcttgttcccccacggcgagaagggctggggcaccgacatcgcactgagactgagagacaactccgtgatcgacaacaacacgagacagaatgtgcgcacccgggtgacccagatgcagtactacggcttccacctgtctgtgcgggatacattcaatcctatcctcaacgcaggcaagctgacacagcagttcatcgtcgacagctacagcaagatggaagccaacagaattaacttcatcaaagccaatcagtctaagctgagagtcgaaaaatatagcggcctgatggactacctgaagagcagatccgaaaacgacaacgtgcctatcggtaagatgattatcctgccctctagtttcgaaggcagccctagaaacatgcagcagcggtaccaggatgccatggccatcgtgaccaaatacggcaagcctgacctgtttatcacaatgacctgtaaccctaagtgggccgatatcaccaacaaccttcaaagatggcagaaagtggaaaatcgtcctgatctggtggccagagtgttcaacatcaaacttaacgccctgctcaatgatatctgcaagttccacctgtttggcaaggtgatcgccaagatccacgtgatcgagttccagaagagaggcctgcctcacgcccacatcctgctcatcctggacagcgagagcaagctgcggtctgaggacgatattgacagaatagtgaaagccgagatccctgacgaggaccagtgccctagactgtttcagatcgtgaagagcaacatggtgcacggcccctgtggtatccagaatcctaactccccttgcatggaaaacggcaaatgcagcaagggatatcccaaggaatttcagaacgccaccatcggcaacatagacggctaccccaagtacaagagaagaagcggtagcacaatgagcattggaaacaaggttgtcgacaacacctggatcgttccttacaacccttacctgtgcctgaagtacaactgccacatcaacgtggaagtgtgtgccagtatcaagtctgtgaagtacctgttcaagtacatctacaaaggccacgactgcgcgaacatccaaataagcgagaaaaacatcatcaaccacgatgaagtgcaggactttatcgacagtagatacgtgtccgcccctgaggccgtgtggagactgttcgccatgcggatgcacgaccaatctcacgccatcacccgtctggctattcacctgcccaacgaccagaacttgtacttccataccgatgatttcgccgaggtgctggacagagccaaaaggcacaacagcaccctgatggcttggttccttctgaacagggaagatagtgacgcaagaaattactactactgggagatcccacagcactacgtgttcaacaacagcctgtggaccaagcgcagaaagggcgggaacaaggtcctgggcagactgttcaccgtctctttccgggaaccggaacggtactacctgagactgctgctgctgcacgtgaagggcgccatcagctttgaagatctgcggacagtcggcggcgtgacgtacgacaccttccacgaggccgctaaacaccgtggactgctgctcgatgacaccatctggaaggacaccatcgacgacgccatcattctgaatatgcccaagcagctgcggcagctgttcgcctacatctgcgtatttggctgcccctcagccgccgacaagctgtgggacgaaaacaagtcccatttcatcgaggacttctgctggaagctgcacagacgggaaggtgcctgcgtgaactgtgaaatgcacgccctgaatgagatccaggaggtgttcacactgcatggaatgaaatgcagccacttcaagctccctgactaccccctgctgatgaacgccaacacatgcgaccagctgtacgagcagcagcaggccgaggtgctgatcaacagcctgaacgatgagcagctggccgcgttccaaacaatcacgagcgccatcgaggaccagaccgtgcaccccaaatgcttcttcctggacggccctggtggcagcggcaagacatacctgtacaaagtgctgacccactacatccggggcagaggaggcaccgtgctgcccacagccagcaccggcatcgccgctaatctcctgctgggcggacggaccttccactctcagtacaagctaccaattcctctgaatgagacatctataagcagactggacatcaaaagcgaggtggccaagaccatcaagaaggcccagctgctgatcattgatgagtgcaccatggctagctcccacgccatcaacgccatcgaccggttgttaagagagatcatgaaccttaacgtggccttcggaggcaaggttctgctgcttggaggcgactttcggcagtgtctgagcatcgtgcctcacgccatgagaagcgccatcgtccagacctccctgaagtattgtaatgtgtggggctgtttcaggaagctgagcctaaagaccaacatgagaagcgaggactccgcctactccgaatggctggtgaagctgggcgacggcaagctggacagcagcttccatctgggcatggacatcatcgagatcccccatgaaatgatctgcaacggctcgatcatcgaagctacgttcggaaattccatctccatcgacaacatcaaaaacatcagcaaacgcgctatcctgtgtcctaaaaacgagcacgtgcagaagctgaacgaggaaatcctggacattctggacggagattttcacacatacctgtcagatgattccatcgacagcaccgacgacgccgagaaggaaaatttccctatcgagttcctgaacagcatcacccctagcggcatgccttgtcacaagctgaaactgaaggtgggggctatcatcatgctcctgagaaacctgaacagcaagtggggactgtgcaacggcaccagattcatcatcaagagactgcggcctaacatcatcgaggctgaagtattgaccggctctgctgagggcgaggtggtgctgatccctagaattgacctgagtcctagcgacacaggcctgccattcaagctgatcagacggcaatttcccgtgatgcctgctttcgccatgacaatcaacaagtctcagggccagacactggatcgggtgggcatcttcctgcctgagcctgtgttcgcccacggccagctgtatgtggccttcagccgggtgagaagagcctgtgacgtgaaggtgaaggtggttaatacctccagccagggcaaactggtgaagcacagcgaaagcgtgttcacgctgaacgtggtgtaccgcgagatcctagaatgataactcgag

SEQ ID NO: 24 - protein, 1496 aa, Artificial sequence
  Helraiser transposase amino acid sequence
MSKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 25 - DNA, 150 bp, Artificial sequence
  Helitron LTS left terminal sequence
tcctatataataaaagagaaacatgcaaattgaccatccctccgctacgctcaagccacgcccaccagccaatcagaagtgactatgcaaattaacccaacaaagatggcagttaaatttgcatacgcaggtgtcaagcgccccaggagg

SEQ ID NO: 26 - DNA, 150 bp, Artificial sequence
  Helitron RTS right terminal sequence
aaatttatgtattattttcatatacattttactcatttcctttcatctctcacacttctattatagagaaagggcaaatagcaatattaaaatatttcctctaattaattccctttcaatgtgcacgaatttcgtgcaccgggccactag

SEQ ID NO: 27 - DNA, 875 bp, Artificial sequence
  Helitron transposon plasmid clone sequence
aggccttgtcttccatatcctatataataaaagagaaacatgcaaattgaccatccctccgctacgctcaagccacgcccaccagccaatcagaagtgactatgcaaattaacccaacaaagatggcagttaaatttgcatacgcaggtgtcaagcgccccaggaggcaacggcggccgcgggctcccaggaccttcgctggccccgggaggcgaggccggccgcgcctagccacacccgcgggctcccgggaccttcgccagcagagagcagagcgggagagcgggcggagagcgggaggtttggaggacttggcagagcaggaggccgctggacatagagcagagcgagagagagggtggcttggagggcgtggctccctctgtcaccccagcttcctcatcacagctgtggaaactgacagcagggaggaggaagtcccacccccacagaatcagccagaatcagccgttggtcagacagctctcagcggcctgacagccaggactctcattcacctgcatctcagaccgtgacagtagagaggtgggactcctgcaggtacgatcaagcggcgcgcctttaatcactttatcagtcattgtttgcatcaatgttgtttttatatcatgtttttgttgtttttatatcatgtctttgttgttgttatatcatgttgttattgtttatttattaataaatttatgtattattttcatatacattttactcatttcctttcatctctcacacttctattatagagaaagggcaaatagcaatattaaaatatttcctctaattaattccctttcaatgtgcacgaatttcgtgcaccgggccactagtatttaaataaaaagcaaagcgatcgc

SEQ ID NO: 28 - DNA, 1048 bp, Artificial sequence
  ZB transposase plasmid clone sequence
aatcgatgccaccatgatgggcaagaacaaagagctgagccaagacctgcggagcctgatcgtggaaaagcatttcgacggcaacggttatcggagaatcagcagaatgctgaatgtgcccgtctccaccgtgggcgctatcatccggaagtggaagaagcacaaattcaccatcaacagacctagatctggcgcccctagaaagatccctgtgcggggcgtgcagcggatcatcagacgggtgctgcaagagcctagaacaaccagagctgaactgcaggaagatctggcctctgccggcacaatcgtgtccaagaagacaatttctaatgccctgaaccaccacggcatccacgcccggagccctcggaaaacccctctgctgaacaagaaacacgtggaagccagactgaaattcgccaaacagcacctggagaagcctgtggactactgggagacaattgtgtggagcgacgagagcaagatcgagttgtttggaagccacagcacccaccacgtctggagaaggaacggcaccgcccaccatcctaagaacacaatccccaccgttaagttcggaggcggcagcatcatggtgtggggctgttttagcgctagaggaaccggcaggctgcacatcatcgagggaagaatgaacggcgagatgtacagagatatcctggataagaacctgctgccttccacaagaaaactgaagatgaagcgcggctggaccttccagcaggataacgaccccaagcacaaggccaaggaaaccatgaaatggttccagagaaagaaaatcaagctgctggagtggcctagccagagcccagacctgaacccaatcgagaacctgtggcgggaactgaagatcaaggtgcacaagagaggcccccggaatctgcaggacctgaagaccgtgtgcgtggaggaatgggccagaatcacccccgagcagtgcagaagactggtgtctccatacaagcggagactggaagccgtgatcaccaacaagggcttcagcacaaagtactgataactcgag

SEQ ID NO: 29 - protein, 341 aa, Artificial sequence
  ZB transposase amino acid sequence
MMGKNKELSQDLRSLIVEKHFDGNGYRRISRMLNVPVSTVGAIIRKWKKHKFTINRPRSGAPRKIPVRGVQRIIRRVLQEPRTTRAELQEDLASAGTIVSKKTISNALNHHGIHARSPRKTPLLNKKHVEARLKFAKQHLEKPVDYWETIVWSDESKIELFGSHSTHHVWRRNGTAHHPKNTIPTVKFGGGSIMVWGCFSARGTGRLHIIEGRMNGEMYRDILDKNLLPSTRKLKMKRGWTFQQDNDPKHKAKETMKWFQRKKIKLLEWPSQSPDLNPIENLWRELKIKVHKRGPRNLQDLKTVCVEEWARITPEQCRRLVSPYKRRLEAVITNKGFSTKY

SEQ ID NO: 30 - DNA, 201 bp, Artificial sequence
  ZB transposon terminal inverted repeat
cagcggggaaaataagtatttgacacatcagcatttttatcagtaaggggatttctaagtgggctactgacacaaaattcctaccagatgtagccatcaagccaaatattgaattcatacaaagaaatcagaacatttaagtatacaagttgagtcataataaataaagtgaaatgacacagggaataagtattgaacaca

SEQ ID NO: 31 - DNA, 455 bp, Artificial sequence
  ZB transposon plasmid clone sequence
gcggccgcatatacagcggggaaaataagtatttgacacatcagcatttttatcagtaaggggatttctaagtgggctactgacacaaaattcctaccagatgtagccatcaagccaaatattgaattcatacaaagaaatcagaacatttaagtatacaagttgagtcataataaataaagtgaaatgacacagggaataagtattgaacacacctgcaggtacgatcaagcggcgcgcctgtgttcaatacttattccctgtgtcatttcactttatttattatgactcaacttgtatacttaaatgttctgatttctttgtatgaattcaatatttggcttgatggctacatctggtaggaattttgtgtcagtagcccacttagaaatccccttactgataaaaatgctgatgtgtcaaatacttattttccccgctgtatatgcgatcgc

SEQ ID NO: 32 - DNA, 1048 bp, Artificial sequence
  Intruder (IT) transposase plasmid clone sequence
aatcgatgccaccatggccagactgagcaccgccacaagacacaaggtggtgatcctgcaccagcaaggactttcccaggccgaaatcagccggcagaccggcgtgtccagatgcgccgtgcaggccctgctgaagaaacataaggaaaccggcaatgtggaagatagacggcggagcggcagacctagaaagctgacagctgccgacgagcgccacatcatgctgaccagcctgcggaaccggaagatgagctccagcgccatcagcagcgagctggctgagaacagcggtacactggtgcaccctagcacagtgcgcagatctctggtcagatctggactgcacggcagactggccgctaagaagccttacctgagaaggggcaacaaggccaaaagactgaattacgccagaaagcacaggaactggggcgccgaaaagtggcagcaggttctgtggaccgacgagtctaagttcgagattttcggatgcagcagaagacagttcgtgcggcggcgggccggcgagagatacaccaacgagtgcctgcaggccaccgtgaagcatggcggaggatctctgcaggtgtggggctgtatcagcgctaatggcgtgggcgacctcgtgcggatcaacggcctgctgaacgccgagaagtacagacagatcctgatccaccacgccatcccctctggcagacacctgatcggccctaagttcatcctgcaacacgacaacgaccccaagcacaccgctaaagtgattaagaactacctgcagagaaaagaggaacagggcgtgctggaagtgatggtgtggcccccccagagccctgatctgaacatcatcgagagcgtgtgggattacatgaagcgggaaaaacagctgagactgcctaaaagcaccgaggaactgtggctggtcctgcaggacgtgtgggccaacctgccagctgagtttctgcaaaagctgtgcgccagcgtgcctcggagaatcgacgccgtgctgaaggccaagggcggccacacaaagtattgataactcgag

SEQ ID NO: 33 - protein, 341 aa, Artificial sequence
  Intruder (IT) transposase amino acid sequence
MARLSTATRHKVVILHQQGLSQAEISRQTGVSRCAVQALLKKHKETGNVEDRRRSGRPRKLTAADERHIMLTSLRNRKMSSSAISSELAENSGTLVHPSTVRRSLVRSGLHGRLAAKKPYLRRGNKAKRLNYARKHRNWGAEKWQQVLWTDESKFEIFGCSRRQFVRRRAGERYTNECLQATVKHGGGSLQVWGCISANGVGDLVRINGLLNAEKYRQILIHHAIPSGRHLIGPKFILQHDNDPKHTAKVIKNYLQRKEEQGVLEVMVWPPQSPDLNIIESVWDYMKREKQLRLPKSTEELWLVLQDVWANLPAEFLQKLCASVPRRIDAVLKAKGGHTKY

SEQ ID NO: 34 - DNA, 210 bp, Artificial sequence
  Intruder (IT) transposon terminal inverted repeat
cagtactgtgcaaaagttttaggcaggtgtgaaaaaatgctgtaaagtaagaatgctttcaaaaatagacatgttaatagtttatatttatcaattaacaaaatgcaaagtgagtgaacagaagaaaaatctacatcaaatcaatatttggtgtgaccaccctttgccttcaaaacagcatcaattcttctaggtacacttgcacacagt

SEQ ID NO: 35 - DNA, 467 bp, Artificial sequence
  Intruder (IT) transposon plasmid clone sequence
gcggccgctacagtactgtgcaaaagttttaggcaggtgtgaaaaaatgctgtaaagtaagaatgctttcaaaaatagacatgttaatagtttatatttatcaattaacaaaatgcaaagtgagtgaacagaagaaaaatctacatcaaatcaatatttggtgtgaccaccctttgccttcaaaacagcatcaattcttctaggtacacttgcacacagtcctgcaggtacgatcaagcggcgcgccactgtgtgcaagtgtacctagaagaattgatgctgttttgaaggcaaagggtggtcacaccaaatattgatttgatgtagatttttcttctgttcactcactttgcattttgttaattgataaatataaactattaacatgtctatttttgaaagcattcttactttacagcattttttcacacctgcctaaaacttttgcacagtactgtagcgatcgc

SEQ ID NO: 36 - DNA, 1831 bp, Artificial sequence
  SPINon transposase plasmid clone sequence
aatcgatgccaccatgaccatggacagagtcgagaagaacgtgaagaagcggaagtacagcgaggatttccttcagtacggctttaccagcatcatcacagctggcatcgagaagcctcaatgtgtgatctgctgcgaggtgctgtctgctgaaagcatgaagcctaacaagctgaagcggcacttcgacagcaagcaccccagcttcgccggaaaggacaccaactactttagatccaaggccgatggcctgaaaaaggccagactggatacaggcggcaagtaccacaaacagaacgtggccgccatcgaagcctcttacctggtggccctgagaatcgcccgagctatgaagccccacacaatcgccgaggacctgctgctgcctgctgccaaggacatcgtgagggtgatgatcggagatgaattcgtgaccaagctgagcgcaatctctctgtccaacgacaccgtgcggagaagaattgacgacatgagcgccgacatcctggaccaggtgatccaggaaatcaagtctgccccactgcccatcttcagcatccagctggacgaatctaccgacgtggccaactgcagccagctgctggtgtacgtgcggtacatcaatgacggcgacttcaaggacgagttcctgttctgcaagcccctggaaatgacaaccaccgccagagacgttttcgacacagtgggcagctttctgaaggaacacaagatcagctgggaaaaggtctgtggcgtgtgcaccgatggagcccccgccatgctgggctgcagatcaggcttccaaagactggtactgaatgagagccctaaggtgatcggcacccactgcatgatccacagacagatcctggccaccaaaacactcccacaggagctgcaagaggtgatgaagagcgtgatttcctccgtgaacttcgtgaaggccagcaccctgaacagcagactgtttagccagctgtgcaacgagctcgacgcccctaacaacgccctgctctttcacaccgaggtgcgctggctgtccagaggcaaggtgctgaaacgggtgttcgagctcagagatgaactgaagaccttcttcaaccagaaagccagacctcagttcgaggccctgttcagcgacaagagcgagctgcagaaaatcgcctacctggtggacatcttcgccattctgaatgagctgaacctgtccctgcagggccctaatgccacatgtctggatctgagcgagaagatcagatcttttcagatgaaactgcagctgtggcagaagaagctggatgagaacaagatctacatgctgcctacactgagcgcttttttcgaggaacatgacatcgagcctgacaagcggatcaccatgatcatcagcgtgaaagagcacctgcacatgctggctgatgagatcagcagctacttcccaaatctgccagatacacctttcgccctggccagatctcctttcactgtgaaggtggaagatgtgcccgagaccgctcaggaggaattcatcgagctgatcaacagcgacgccgctaggaccgacttcagcaccatgcctgtgaccaagttctggatcaagtgcctgcagtcttatcctgtgctgagcgaaaccgtgctgcggctgctgctgcccttccccaccacctacctgtgtgaaacaggattttcttctctgctcgttatcaagtcgaaatatagaagccggctggtggtcgaagatgaccttcggtgcgcgctggccaaaacagcccctagaatctccgatctggtccggaagaaacagagccagcctagccactgataactcgag

SEQ ID NO: 37 - protein, 602 aa, Artificial sequence
  SPINon transposase amino acid sequence
MTMDRVEKNVKKRKYSEDFLQYGFTSIITAGIEKPQCVICCEVLSAESMKPNKLKRHFDSKHPSFAGKDTNYFRSKADGLKKARLDTGGKYHKQNVAAIEASYLVALRIARAMKPHTIAEDLLLPAAKDIVRVMIGDEFVTKLSAISLSNDTVRRRIDDMSADILDQVIQEIKSAPLPIFSIQLDESTDVANCSQLLVYVRYINDGDFKDEFLFCKPLEMTTTARDVFDTVGSFLKEHKISWEKVCGVCTDGAPAMLGCRSGFQRLVLNESPKVIGTHCMIHRQILATKTLPQELQEVMKSVISSVNFVKASTLNSRLFSQLCNELDAPNNALLFHTEVRWLSRGKVLKRVFELRDELKTFFNQKARPQFEALFSDKSELQKIAYLVDIFAILNELNLSLQGPNATCLDLSEKIRSFQMKLQLWQKKLDENKIYMLPTLSAFFEEHDIEPDKRITMIISVKEHLHMLADEISSYFPNLPDTPFALARSPFTVKVEDVPETAQEEFIELINSDAARTDFSTMPVTKFWIKCLQSYPVLSETVLRLLLPFPTTYLCETGFSSLLVIKSKYRSRLVVEDDLRCALAKTAPRISDLVRKKQSQPSH

SEQ ID NO: 38 - DNA, 250 bp, Artificial sequence
  SPINon transposon 5′ terminal inverted repeat
cagcggttctcaacctgtgggtcgcgacccctttgggggtcaaacgaccctttcacaggggtcgcctaagaccatcggaaaacacatatttccgatggtcttaggaaccgagacaccgctcctctatccgtctccaggcgggtccgcccacatgcagatacgcccacatabgagtacccggcgtgatgacatcatcgcgccaaccccatcacatacaccccgtacaaatacaggtgtatgtgacagggtt

SEQ ID NO: 39 - DNA, 250 bp, Artificial sequence
  SPINon transposon 3′ terminal inverted repeat
gtaattagaaataaatatttcacaatatataattacatattgtttttgtgattaatcactatgctttaattatgttcaatttgtaacaatgaaaatacatcctgcatatcagatatttacattacgattcataacagtagcaaaattacagttatgaagtagcaacgaaaataattttatggttgggggtcaccacaacatgaggaactgtattaaagggtcgcggcattaggaaggttgagaaccactg

SEQ ID NO: 40 - DNA, 808 bp, Artificial sequence
  SPINon transposon plasmid clone sequence
gcggccgcctttaacacagcggttctcaacctgtgggtcgcgacccctttgggggtcaaacgaccctttcacaggggtcgcctaagaccatcggaaaacacatatttccgatggtcttaggaaccgagacaccgctcctctatccgtctccaggcgggtccgcccacatgcagatacgcccacatacgagtacccggcgtgatgacatcatcgcgccaaccccatcacatacaccccgtacaaatacaggtgtatgtgacagggttggcgccataatgtacttatgcggaccagtcacacatgtgtagagagcagctactgtgtagaaagcagctactgtgttgaaagcagctactctgttaaaagcagctactgtgttgaaagcagcagtattggaggtaaaacctgcaggtacgatcaagcggcgcgcccgttggctttttacgcatactgttgcaaaatgtagcaatgtagtttactgttgttatattaagactgttacccatgctacaccatgcttcaagacaaaatttcatttatttgtaattagaaataaatatttcacaatatataattacatattgtttttgtgattaatcactatgctttaattatgttcaatttgtaacaatgaaaatacatcctgcatatcagatatttacattacgattcataacagtagcaaaattacagttatgaagtagcaacgaaaataattttatggttgggggtcaccacaacatgaggaactgtattaaagggtcgcggcattaggaaggttgagaaccactgctttaacagcgatcgc

SEQ ID NO: 41 - DNA, 1933 bp, Artificial sequence
  TcBusterM3 (D189A/V377T/E469K) transposase plasmid clone sequence
aatcgatgccaccatgatgctgaattggctgaagtctggaaagctggaaagccagagccaggagcagagcagctgctacctggaaaattctaactgcctgccccccacactggacagcaccgacatcatcggcgaagagaacaaggccggtacaaccagccggaagaaaagaaagtacgacgaggactacctgaacttcggattcacctggaccggcgacaaggacgagcctaacggcctgtgtgtgatctgcgaacaggtggtgaacaacagctccctgaatcctgccaaactgaaacggcacctggatacgaaacaccctaccctgaagggcaagtcagagtacttcaagagaaagtgcaacgagctgaaccagaaaaagcacacctttgagagatacgtcagagatgacaacaagaatctgttaaaagcctcttatctggtgtccctgagaatcgctaagcagggcgaagcgtacaccatcgccgagaaactgatcaagccttgcaccaaggacctgaccacctgcgtgttcggcgagaagttcgcaagcaaggtggacctggtgcccctgagcgccaccaccatcagccgaagaatcgaggacatgagctacttctgtgaggccgtgctggtcaacaggctgaagaatgccaagtgtggctttacccttcagatggacgagtccaccgacgtggccggcctggctatcctgctggtgtttgtgcggtacatccacgagagctctttcgaagaggatatgctgttctgcaaggccctgcctacacagaccacaggcgaggaaatcttcaacctgctgaacgcttactttgagaagcacagcatcccttggaacctgtgttaccacatctgtaccgatggagccaaggctatggtgggcgtgatcaagggagttatcgctagaatcaagaagctggtgccagatatcaaagctagccactgctgcctgcacagacacgccctggccgtcaagcggatccccaatgccctgcatgaggtgctgaacgatgccgtgaagatgatcaacttcatcaagtctagacctctgaacgccagagtgttcgccctgctgtgcgacgatctgggctccctgcataagaacctgctgctgcacacagaaacccggtggctcagccggggcaaggtgctgacaagattctgggagctgagagacgagatccgcatctttttcaacgagcgggagttcgccggcaagctgaacgacaccagctggctgcagaacctagcctacatcgccgatatcttcagctatctgaatgaagtgaacctgagcctgcaaggccctaattccaccattttcaaggtgaacagcagaatcaatagcatcaaaagcaagctgaagctctgggaggaatgcatcacaaagaacaacaccaagtgcttcgccaacctgaacgatttcttagaaacctccaacacagccctggaccccaatctcaaaagcaatatcctggagcacctgaacggcctgaaaaacaccttcctggaatactttccacctacatgcaacaacatcagctgggtggaaaaccccttcaacgagtgcggcaacgtggacacactgcctatcaaggaacgggagcagctaatcgacatcagaaccgacaccacactgaagtcttcttttgttcctgacggcatcggacctttctggatcaagctgatggatgaattccccgagatttctaagagagccgtgaaggaactgatgccttttgtgaccacttacctgtgcgagaagagcttcagcgtgtacgtggccacaaagaccaaatacagaaaccggctggacgccgaggacgacatgagactgcaactgaccacaatccacccagatattgataacctgtgtaacaacaaacaggcccagaagtctcactgataactcgag

SEQ ID NO: 42 - protein, 636 aa, Artificial sequence
  TcBuster transposase TcBusterCO amino acid sequence
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLKNAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 43 - DNA, 328 bp, Artificial sequence
  TcBuster transposon 5′ terminal inverted repeat #1
cagtgttcttcaacctgtgttccgcggaaccctagggttccacccaaaggctttcggggttccgcgagtcattgcttcaattcgagagacgtcggccgcgccgctcttcagaatgcacatgcgtcaatcggagtttcatgttgaaacatgttatccattcgcatagttgacttacactgcacttaaccttaattttcaaaaatatgtaactgtacttgtggtcgtagttttgttgttgtttttggtttagacaagcaaaggtaagttaacttacagttttaaaataaattgtattttgtttgatcctaacctagaatcgttcagaaat

SEQ ID NO: 44 - DNA, 458 bp, Artificial sequence
  TcBuster transposon 3′ terminal inverted repeat #1
tatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggctagaccaaagcacgggctcaccttgttcgtaacaagtcaacgcagctgtccctaaaatctcatctgggtgtattactaaatgaagggttccataaaaaaaaatatctcgacaaagggttccgccggatggcaaaggttgaagaacactg

SEQ ID NO: 45 - DNA, 845 bp, Artificial sequence
  TcBuster transposon plasmid clone sequence #1
gcggccgctcctagatcagtgttcttcaacctgtgttccgcggaaccctagggttccacccaaaggctttcggggttccgcgagtcattgcttcaattcgagagacgtcggccgcgccgctcttcagaatgcacatgcgtcaatcggagtttcatgttgaaacatgttatccattcgcatagttgacttacactgcacttaaccttaattttcaaaaatatgtaactgtacttgtggtcgtagttttgttgttgtttttggtttagacaagcaaaggtaagttaacttacagttttaaaataaattgtattttgtttgatcctaacctagaatcgttcagaaatcctgcaggtacgatcaagcggcgcgcctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggctagaccaaagcacgggctcaccttgttcgtaacaagtcaacgcagctgtccctaaaatctcatctgggtgtattactaaatgaagggttccataaaaaaaaatatctcgacaaagggttccgccggatggcaaaggttgaagaacactgtcctagatgcgatcgc

SEQ ID NO: 46 - DNA, 328 bp, Artificial sequence
  TcBuster transposon 5′ terminal inverted repeat #2
cagtgttcttcaacctgtgttccgcggaaccctagggttccacccaaaggctttcggggttccgcgagtcattgcttcaattcgagagacgtcggccgcgccgctcttcagaatgcacatgcgtcaatcggagtttcatgttgaaacatgttatccattcgcatagttgacttacactgcacttaaccttaattttcaaaaatatgtaactgtacttgtggtcgtagttttgttgttgttttaggtttagacaagcaaaggtaagttaacttacagttttaaaataaattgtattttgtttgatcctaacctagaatcgttcagaaat

SEQ ID NO: 47 - DNA, 250 bp, Artificial sequence
  TcBuster transposon 3′ terminal inverted repeat #2
tttttattttttattttatatattattttatttggaatatttatctttttgtttaaagtttgtttaaataaatgcaaatttaaatcttatttagagtttttattaccaaagcacgggctcaccttgttcgtaacaagtcaacgcagctgtccctaaaatctcatctgggtgtattactaaatgaagggttccataaaaaaaaatatctcgacaaagggttccgccggatggcaaaggttgaagaacactg

SEQ ID NO: 48 - DNA, 637 bp, Artificial sequence
  TcBuster transposon plasmid clone sequence #2
gcggccgctcctagatcagtgttcttcaacctgtgttccgcggaaccctagggttccacccaaaggctttcggggttccgcgagtcattgcttcaattcgagagacgtcggccgcgccgctcttcagaatgcacatgcgtcaatcggagtttcatgttgaaacatgttatccattcgcatagttgacttacactgcacttaaccttaattttcaaaaatatgtaactgtacttgtggtcgtagttttgttgttgttttaggtttagacaagcaaaggtaagttaacttacagttttaaaataaattgtattttgtttgatcctaacctagaatcgttcagaaatcctgcaggtacgatcaagcggcgcgcctttttattttttattttatatattattttatttggaatatttatctttttgtttaaagtttgtttaaataaatgcaaatttaaatcttatttagagtttttattaccaaagcacgggctcaccttgttcgtaacaagtcaacgcagctgtccctaaaatctcatctgggtgtattactaaatgaagggttccataaaaaaaaatatctcgacaaagggttccgccggatggcaaaggttgaagaacactgtcctagatgcgatcgc

SEQ ID NO: 49 - DNA, 1042 bp, Artificial sequence
  Passport (PT) transposase plasmid clone sequence
aatcgatgccaccatgaagacaaaggaactgacaaagcaagtgagagataaggtggtggaaaagtatgaggccggcctgggctacaagaagatcagcagagccctgaatatcagcctgtctacaattaagtctatcatccggaagtggaaagagtacggcaccacagccaacctgcctagaggcggacggccaccaaagctcaagagcagaacccggagaaaactcatcagagaggccacaagacggcctatggtcaccctggaagagctgcagagaagcacagccgaggtgggcgagtctgtgcaccggaccaccatctcccgcctgctgcataagtccggcctgtacggcagagtggcccggcggaagcctctgctgaaaggcatccacaagaagagcagactggaattcgccagaagccacgtgggcgacaccgctaacatgtggaagaaagtgctgtggagcgacgagacaaagatcgagctgtttggcctgaacgctaagcggtacgtgtggcggaaacctaacaccgcccaccaccccgagcacaccatccccaccgtgaaacacggaggaggaagcattatgctgtggggctgcttcagcagcgccggaaccggcaagctggttagaatcgagggcaaaatggacggcgccaagtaccgggaaatcctggaagagaacctgatgcagtccgccaaagacctgagactgggcagaaggtttatcttccagcaggacaacgaccccaagcacactgctagagccaccaaggaatggttcggactgaagaacgtgaacgtgctgaagtggcctagccagagccctgatctgaatcctatcgagaatctgtggcaggatctgaagatcgctgtccacagaagatctcctagcaacctgaccgagctgcacctgttctgccaggaggaatggaccaacctcagcatctctaggtgtgccaagctggtggaaacctaccccaagagactggccgccgtgatcgccgctaagggcggcagcaccaaatactgataactcgag

SEQ ID NO: 50 - protein, 339 aa, Artificial sequence
  Passport (PT) transposase amino acid sequence
MKTKELTKQVRDKVVEKYEAGLGYKKISRALNISLSTIKSIIRKWKEYGTTANLPRGGRPPKLKSRTRRKLIREATRRPMVTLEELQRSTAEVGESVHRTTISRLLHKSGLYGRVARRKPLLKGIHKKSRLEFARSHVGDTANMWKKVLWSDETKIELFGLNAKRYVWRKPNTAHHPEHTIPTVKHGGGSIMLWGCFSSAGTGKLVRIEGKMDGAKYREILEENLMQSAKDLRLGRRFIFQQDNDPKHTARATKEWFGLKNVNVLKWPSQSPDLNPIENLWQDLKIAVHRRSPSNLTELHLFCQEEWTNLSISRCAKLVETYPKRLAAVIAAKGGSTKY

SEQ ID NO: 51 - DNA, 385 bp, Artificial sequence
  Passport (PT) transposon 5′ terminal inverted repeat
tacagtgccttgcataagtattcaccccctttggacttttctacattttgtcatgctataaccacagattaaaatttatttcatcgtgagtttatgtaatggaccaacacaaaatagtgcatcatttggaagtggggggaaatattacatggatttcacaattatttacaaataaaaatctgaaaagtgttgagtgcatatgtattcaccccctttactgtgaaacccctaacaaagatctggtgcgaccaattgcattcacaagtcacatttgcaagtcacataattagtaaatagggtccacctgtctgcaatttaatctcagtataaatacacctgttctgtgacggactcagagtttgttggagatcattactgaacaaac

SEQ ID NO: 52 - DNA, 237 bp, Artificial sequence
  Passport (PT) transposon 3′ terminal inverted repeat
ggttctaccaagtattgacacaggggggtgaatacttatgcacccaacagatgtcaacttttttgttctcattattgtttgtgtcacaataaaatttattttgcacctccaaagtactatgcatgttttgttgatcaaacgggaaaaagtttatttaagtctatttgaattccagttagtaacagtacataatgggaaaaagtccaaggggggtgaatacttatgcaaggcactgta

SEQ ID NO: 53 - DNA, 669 bp, Artificial sequence
  Passport (PT) transposon plasmid clone sequence
gcggccgctatacagtgccttgcataagtattcaccccctttggacttttctacattttgtcatgctataaccacagattaaaatttatttcatcgtgagtttatgtaatggaccaacacaaaatagtgcatcatttggaagtggggggaaatattacatggatttcacaattatttacaaataaaaatctgaaaagtgttgagtgcatatgtattcaccccctttactgtgaaacccctaacaaagatctggtgcgaccaattgcattcacaagtcacatttgcaagtcacataattagtaaatagggtccacctgtctgcaatttaatctcagtataaatacacctgttctgtgacggactcagagtttgttggagatcattactgaacaaaccctgcaggtacgatcaagcggcgcgccggttctaccaagtattgacacaggggggtgaatacttatgcacccaacagatgtcaacttttttgttctcattattgtttgtgtcacaataaaatttattttgcacctccaaagtactatgcatgttttgttgatcaaacgggaaaaagtttatttaagtctatttgaattccagttagtaacagtacataatgggaaaaagtccaaggggggtgaatacttatgcaaggcactgtatagcgatcgc

SEQ ID NO: 54 - DNA, 1903 bp, Artificial sequence
  Bombyx mori yabusame-1 transposase #1 plasmid clone sequence
aatcgatgccaccatggcccctaagaaaaagagaaaggtgggcatccacggcgtgcccgctgccgacatcgagagacaggaagagagaattagagccatgctggaagaggagctgtcagattacagcgatgaatccagcagcgaagatgaaacagatcactgtagcgagcacgaggtgaattacgacaccgaggaagagagaatcgactccgtcgacgtgccatctaattctagacaagaagaggccaatgccatcatcgccaacgaatccgacagcgaccctgatgatgatctgcctctgagcctcgtgcggcagagagcctccgcctctagacaggtgtccggccccttctatacaagcaaggacggcaccaagtggtacaagaattgccagcgcccaaatgtgcggctgagatctgagaacatcgtgacagaacaggcccaggtgaagaatatagccagagatgccagcaccgaatacgagtgctggaacatcttcgtgaccagcgatatgctgcaggaaatcctgacccacaccaactccagcatcagacaccggcagaccaagaccgccgctgagaacagcagcgccgagacaagcttctacatgcaggagaccaccctgtgtgaactgaaggccctgatcgctctgctgtacctggccgggctgatcaagagcaacagacagtctctcaaggacctgtggcggacagacggcaccggcgtggacatctttcggacaacaatgtccctgcagagattccagtttctgcagaacaacatcaggttcgacgacaagagcacgagagacgagagaaagcaaaccgacaacatggctgccttccgcagcatcttcgaccagttcgtgcagtgctgccagaatgcctactctcctagcgagttcctaacaatcgacgagatgctgctgagcttccggggcagatgcctgtttcgggtgtacatccctaacaaacctgctaagtacggcatcaagatcctggccctggtggacgccaagaatttctacgtggtcaacctggaagtctacgctggcaagcagcccagcggaccttacgccgtgagcaacagacctttcgaggttgtggaaaggctgattcagcccgtggccagaagccaccggaacgtgaccttcgataactggttcaccggctacgagctgatgctgcatctgctcaacgagtatagactgacctctgtcggaaccgtgagaaagaacaagagacagatccccgagagctttatccgcaccgacagacagcctaacagcagcgttttcggcttccaaaaggacatcaccctggtgtcctacgcccctaagaagaacaaagtggtggtggtgatgagcacaatgcaccacgacaacagcatcgacgagtctaccggagagaagcagaaacctgagatgatcaccttttacaacagcacaaaggccggcgtggacgtggtcgatgagctgtgcgctaactacaacgtgagtagaaattctaagcggtggcccatgaccctcttctatggcgtgctgaacatggccgctatcaacgcctgcatcatctaccggacaaacaaaaacgtgaccatcaaaagaacagagttcatcagaagcctgggcctgtctatgatctacgagcacctgcacagccggaacaagaagaagaacatccccacctacctgcggcaacggattgaaaaacagctgggcgagccttccccaagacacgtgaacgtgcctggaagatacgtgaggtgtcaggactgcccctacaagaaagaccggaagacaaaaagatcttgtaacgcctgtgctaaacctatctgcatggaacatgccaagttcctgtgcgaaaactgcgccgaactggacagcagcctgtgataactcgag

SEQ ID NO: 55 - DNA, 1903 bp, Artificial sequence
  Bombyx mori yabusame-1 transposase #2 plasmid clone sequence
aatcgatgccaccatggctcctaaaaagaaaagaaaagtgggcatccacggcgtgcctgccgctgatatcgagcggcaggaggaacggattagagccatgctggaagaagaactgagcgactacagcgacgagtctagttctgaagatgagacagaccactgcagcgaacacgaggtgaactacgacaccgaggaagagagaatcgacagcgtcgatgtgccatctaacagcagacaggaggaggccaacgccatcatcgccaatgaaagcgacagcgaccccgacgacgacctgcctctgagcctggtccggcaacgggccagcgcttctcgccagatgagcggcccgcactacacatctaaggatggcaccaagtggtacaagaactgtcagaggcctaatgtgcggctcagaagcgagaacatcgtgaccgagcaggcccaggtgaagaacatcgccagagacgcctccacagagtacgagtgctggaacatcttcgtgaccagcgatatgctgcaagaaatcctgacccacaccaactctagcatcagatggagacagacaaagaccgccgctgaaaactcctccgcctctaccagcttttacatgcaggagacaaccctgtgtgaactgaaggccctgataggactgctgtacatcgcaggcctgattaagtccaacagacagagcctgaaagacctgtggcggaccgacggcaccggcgtggacattttcagaaccacaatgagcctgcagagattccagtttctgcagaacaacatccggttcgacgataagagcacacgggatgagagaaagcagaccgacaacatggccgccttcagatctatcttcgaccagttcgtgcagagctgccagaatgcctacagtcctagcgaattcctgaccatcgacgagatgctgctgagcttccggggaagatgcctgttcagagtgtacatcccaaacaagcctgccaagtacggcatcaagatccttgctctggtggatgccaagaacttctacgtgaaaaatctggaagtctacgccggcaagcagcccagcggcccttatgctgtgtctaatagacctttcgaggttgtggaaagactgatccagcccgtggccagaagccacagaaatgtgacttttgataactggttcaccggctacgaactgatgctgcatctcctaaacgagtacagactgaccagcgtgggcacagtgcgcaagaataagagacagatccccgagtcattcatcagaacagataggcagcctaactctagcgttttcggcttccaaaaggacatcaccctggtcagctacgcccctaagaagaacaaggtggtggtggtgatgagcaccatgcaccacgacaacagcatcgacgagagcacaggcgagaagcagaaacctgagatgatcaccttttataacagcaccaaggccggagtggacgtggtggacgagctgtgcgccaactataacgtgtccagaaacagcaagagatggcctatgaccctgttttacggagtgctgaacatggccgccatcaacgcctgtatcatctaccggacaaacaaaaacgttacaatcaagcggaccgagttcatcagatccctgggcctgagcatgatctacgagcatctgcacagcagaaacaagaagaaaaatatccctacctacctgcggcaacggatcgagaagcagctgggcgaaccttcccccagacacgtgaacgtgcctggtagatacgtgcggtgccaggactgcccctacaagaaggatagaaaaaccaagcggtcatgcaacgcctgcgccaagcccatctgtatggaacacgccaaattcctgtgcgagaattgcgctgagctggactcccacctgtgataactcgag

SEQ ID NO: 56 - protein, 610 aa, Artificial sequence
  Bombyx mori yabusame-1 transposase #1 amino acid sequence
MDIERQEERIRAMLEEELSDYSDESSSEDETDHCSEHEVNYDTEEERIDSVDVPSNSRQEEANAIIANESDSDPDDDLPLSLVRQRASASRQVSGPFYTSKDGTKWYKNCQRPNVRLRSENIVTEQAQVKNIARDASTEYECWNIFVTSDMLQEILTHTNSSIRHRQTKTAAENSSAETSFYMQETTLCELKALIALLYLAGLIKSNRQSLKDLWRTDGTGVDIFRTTMSLQRFQFLQNNIRFDDKSTRDERKQTDNMAAFRSIFDQFVQCCQNAYSPSEFLTIDEMLLSFRGRCLFRVYIPNKPAKYGIKILALVDAKNFYVVNLEVYAGKQPSGPYAVSNRPFEVVERLIQPVARSHRNVTFDNWFTGYELMLHLLNEYRLTSVGTVRKNKRQIPESFIRTDRQPNSSVFGFQKDITLVSYAPKKNKVVVVMSTMHHDNSIDESTGEKQKPEMITFYNSTKAGVDVVDELCANYNVSRNSKRWPMTLFYGVLNMAAINACIIYRTNKNVTIKRTEFIRSLGLSMIYEHLHSRNKKKNIPTYLRQRIEKQLGEPSPRHVNVPGRYVRCQDCPYKKDRKTKRSCNACAKPICMEHAKFLCENCAELDSSL

SEQ ID NO: 57 - DNA, 221 bp, Artificial sequence
  Bombyx mori yabusame-1 transposon 5′ terminal inverted repeat
cccggcgagcatgaggcagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtat

SEQ ID NO: 58 - DNA, 208 bp, Artificial sequence
  Bombyx mori yabusame-1 transposon 3′ terminal inverted repeat
tttaagaaaaagattaataaataataataatttcataattaaaaacttctttcattgaatgccattaaataaaccattattttacaaaataagatcaacataattgagtaaataataataagaacaatattatagtacaacaaaatatgggtatgtcataccctgccacattcttgatgtaactttttttcacctcatgctcgccggg

SEQ ID NO: 59 - DNA, 794 bp, Artificial sequence
  Bombyx mori yabusame-1 transposon plasmid clone sequence
gcggccgcttaattatcccggcgagcatgaggcagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtattttttttatgtaatttttccgattattaatttcaactgttttattggtatttttatgttatccattgttctttttttatgatttactgtatcggttgtctttcgttcctttagttgagtttttttttattattttcagtttttgatcaaacctgcaggtacgatcaagcggcgcgcctcatatttttagtttaaaaaaataattatatgttttataatgaaaagaatctcattatctttcagtattaggttgatttatattccaaagaataatatttttgttaaattgttgatttttgtaaacctctaaatgtttgttgctaaaattactgtgtttaagaaaaagattaataaataataataatttcataattaaaaacttctttcattgaatgccattaaataaaccattattttacaaaataagatcaacataattgagtaaataataataagaacaatattatagtacaacaaaatatgggtatgtcataccctgccacattcttgatgtaactttttttcacctcatgctcgccgggttatttaagcgatcgc

SEQ ID NO: 60 - DNA, 1840 bp, Artificial sequence
  Xenopus tropicalis Uribo2 transposase #1 plasmid clone sequence
aatcgatgccaccatggcccctaagaaaaagcggaaggtgggcatccacggcgtgcccgccgctgccaagagattctacagcgccgaagaggctgccgcccactgcatggcctcttccagcgaagaattcagcggcagcgacagcgagtacgtgccaccagccagcgagagcgacagcagcaccgaggaaagctggtgcagcagctctactgtgtctgccctggaagaacctatggaagtggacgaggacgtggacgacctggaagatcaggaggctggagatcgggccgacgccgccgctggcggcgagcctgcctggggccctccttgcaacttccccccagagatccctccattcaccaccgtgcccggagttaaggttgataccagcaactttgagcctattaacttcttccagctcttcatgaccgaggccatcctgcaggacatggtgctgtacacaaacgtgtacgccgagcagtacctgacccagaatcctctgcctagatacgccagagcacatgcctggcaccctacagacatcgccgagatgaagcgcttcgtgggcctgacactggccatgggcctgatcaaggccaacagcctggaaagctactgggacaccacaacagtcctgtccatccccgtgttctctgctacaatgagcagaaacagataccagctgctgctgagatttctgcactttaacaacaatgccacagccgtgcctcccgaccaacctggccacgaccggctgcataagctgagacctctgattgattctctgagcgagagattcgccgccgtctacacaccctgccagaacatctgcatcgacgagtccctgctgctgttcaagggaagactgcaatttagacagtacatccccagcaagcgggcccgatacggcatcaagttctacaagctgtgcgagagctccagcggctacacgagctacttcctgatctacgagggcaaggacagcaagctggaccccccaggctgccctcctgatctgaccgtgtcgggcaagatcgtgtgggagctgatcagccccctgttgggccagggcttccacctgtacgtggacaacttctattctagcatccccctgtttaccgccctgtactgcctggatacacctgcttgtggcacaatcaacagaaatcggaagggcctgcctagagccctgctggacaagaaactgaacagaggagaaacctacgccctgcggaaaaacgagctgctcgccatcaagtttttcgataagaagaatgtgttcatgctgaccagcatccacgatgagtcagtgatccgggaacagagagtgggcagacctcctaaaaacaagcctctgtgttccaaggaatacagcaaatacatgggaggcgtggacagaaccgaccagctccaacactactacaacgccaccaggaagaccagagcctggtacaaaaaagtgggaatctacctgatccagatggctttgcggaactcttatatcgtgtacaaggccgctgtgcctggccccaagctttcttactacaagtaccaactgcagatcctgcccgccctgctgttcggcggagtcgaggaacagaccgtgcctgagatgcctccatctgataatgtggccagactgataggaaagcacttcatcgacacactgcctcctacccctggcaaacagcggcctcagaagggctgtaaagtgtgcagaaagcggggcattcgcagagatacccggtattattgtcctaagtgccccagaaaccccggcctgtgcttcaagccctgtttcgagatctaccacacccagctgcactactgataactcgag

SEQ ID NO: 61 - DNA, 1840 bp, Artificial sequence
  Xenopus tropicalis Uribo2 transposase #2 plasmid clone sequence
aatcgatgccaccatggcccctaagaagaagagaaaggtgggcatccacggcgtgcccgccgctgccaagcggttctacagcgccgaagaagccgccgcccattgtatggccagctctagcgaagagttcagcggctctgatagcgagtacgtgcctcctgcttctgagagcgacagctccaccgaggaaagctggtgcagctccagcaccgtgtccgccctggaggagcctatggaagtggacgaggacgtggacgacctggaagatcaggaggctggagatcgggctgatgccgctgccggaggagaacctgcctggggccccccttgtaatttccctccggagatccctccattcacaaccgtgccaggcgttaaggtggataccagcaactttgaacctatcaacttttttcagctgttcatgaccgaggccatcctgcaggacatggtgctgtacacaaacgtgtacgccgagcagtacctgacccagaaccccctgaccagaggcgccagagcccacgcctggcaccccaccgacatctgcgagatgaaacggtttgtgggcctgaccctggcgatgggcctgatcaaggccaacagcctggagagctactgggacaccaccacagtgctgagcatccctgtgttcggcgctacaatgtcaagaaaccggtaccagctgctgctgagattcctgcatttcaacaacaacgccaccgccgtacctccagatcagcctggccacgatagactgcacaagctgagacctctgattgatagcctgtctgaaagattcgccaatgtgtataccccctgccagaacatctgcatcgacgagtctctgctgctgtttaagggcagactgcagttcagacagtacatccccagcaagcgggctagatacggcatcaaattctacaagctgtgtgaaagcagcagcggctacaccagctacttcctgatctacgagggcaaggacagcaagctggacccccctggatgtcctcctgacctgaccgtttctggcaaaatcgtgtgggagctgatcagccctctgctgggccagggcttccacctgtatgtggacaatttctattctagcatccccctgtttacagccctctattgtctgaacaccccagcatgcggcaccatcaacagaaatcggaagggactcccaagagcactgctggataagaaactgaaccggggcgagacatacgccctgagaaagaacgagctgctggccatcaagttcttcgacaaaaaaaacgtgttcatgctgacatccattcacgacgaatccgtgatccgggaacagagagtgggcagaccccctaagaacaagcccctgtgcagcaaggaatacagtaaatacatgggcggcgtggaccggaccgaccaacttcaacactactacaacgccacaagaaaaaccagacactggtacaagaaggtcggaatctacctgatccagatggcccttaggaatagctacatcgtctacaaagccgccgtgcctggccctaagctgagctactacaagtaccagctccagatcctgcctgccctgctgttcggcggagtggaagagcaaacagtgcccgagatgcctgactccgataacgtggccagactgatcggcaagcacttcatcgacaccctgcctcctacacccgggaagcagcggccccagaagggctgcaaggtgtgcagaaagagaggcatcaggcgggacacccggtactactgccccaagtgccctagaaatcctggcctgtgcgagaagccttgcttcgagatctatcacacacagctgcactactgataactcgag

SEQ ID NO: 62 - protein, 589 aa, Artificial sequence
  Xenopus tropicalis Uribo2 transposase #1 amino acid sequence
MAKRFYSAEEAAAHCMASSSEEFSGSDSEYVPPASESDSSTEESWCSSSTVSALEEPMEVDEDVDDLEDQEAGDRADAAAGGEPAWGPPCNFPPEIPPFTTVPGVKVDTSNFEPINFFQLFMTEAILQDMVLYTNVYAEQYLTQNPLPRYARAHAWHPTDIAEMKRFVGLTLAMGLIKANSLESYWDTTTVLSIPVFSATMSRNRYQLLLRFLHFNNNATAVPPDQPGHDRLHKLRPLIDSLSERFAAVYTPCQNICIDESLLLFKGRLQFRQYIPSKRARYGIKFYKLCESSSGYTSYFLIYEGKDSKLDPPGCPPDLTVSGKIVWELISPLLGQGFHLYVDNFYSSIPLFTALYCLDTPACGTINRNRKGLPRALLDKKLNRGETYALRKNELLAIKFFDKKNVFMLTSIHDESVIREQRVGRPPKNKPLCSKEYSKYMGGVDRTDQLQHYYNATRKTRAWYKKVGIYLIQMALRNSYIVYKAAVPGPKLSYYKYQLQILPALLFGGVEEQTVPEMPPSDNVARLIGKHFIDTLPPTPGKQRPQKGCKVCRKRGIRRDTRYYCPKCPRNPGLCFKPCFEIYHTQLHY

SEQ ID NO: 63 - DNA, 181 bp, Artificial sequence
  Xenopus tropicalis Uribo2 transposon 5′ terminal inverted repeat #1
ccctttgcctgccaatcacgcatgggatacgtcgtggcagtaaaagggcttaaatgccaacgacgcgtcccatacgttgttggcattttaagtcttctctctgcagcggcagcatgtgccgccgctgcagagagtttctagcgatgacagcccctctgggcaacgagccgggggggctgtc

SEQ ID NO: 64 - DNA, 234 bp, Artificial sequence
  Xenopus tropicalis Uribo2 transposon 3′ terminal inverted repeat #1
tttgcatttttagacatttagaagcctatatcttgttacagaattggaattacacaaaaattctaccatattttgaaagcttaggttgttctgaaaaaaacaatatattgttttcctgggtaaactaaaagtcccctcgaggaaaggcccctaaagtgaaacagtgcaaaacgttcaaaaactgtctggcaatacaagttccactttgggacaaatcggctggcagtgaaaggg

SEQ ID NO: 65 - DNA, 466 bp, Artificial sequence
  Xenopus tropicalis Uribo2 transposon plasmid clone sequence #1
gcggccgcttaaccctttgcctgccaatcacgcatgggatacgtcgtggcagtaaaagggcttaaatgccaacgacgcgtcccatacgttgttggcattttaagtcttctctctgcagcggcagcatgtgccgccgctgcagagagtttctagcgatgacagcccctctgggcaacgagccgggggggctgtccctgcaggtacgatcaagcggcgcgcctttgcatttttagacatttagaagcctatatcttgttacagaattggaattacacaaaaattctaccatattttgaaagcttaggttgttctgaaaaaaacaatatattgttttcctgggtaaactaaaagtcccctcgaggaaaggcccctaaagtgaaacagtgcaaaacgttcaaaaactgtctggcaatacaagttccactttgggacaaatcggctggcagtgaaagggttaagcgatcgc

SEQ ID NO: 66 - DNA, 96 bp, Artificial sequence
  Xenopus tropicalis Uribo2 transposon 5′ terminal inverted repeat #2
cctttttactgccaatgacgcatgggatacgtcgtggcagtaaaagggcttaaatgccaacgacgcgtcccatacgttgttggcattttaattctt

SEQ ID NO: 67 - DNA, 148 bp, Artificial sequence
  Xenopus tropicalis Uribo2 transposon 3′ terminal inverted repeat #2
ttgttctgaaaaaaacaatatattgttttcctgggtaaactaaaagtcccctcgaggaaaggcccctaaagtgaaacagtgcaaaacgttcaaaaactgtctggcaatacaagttccactttgaccaaaacggctggcagtaaaaggg

SEQ ID NO: 68 - DNA, 465 bp, Artificial sequence
  Xenopus tropicalis Uribo2 transposon plasmid clone sequence #2
gcggccgcttaacctttttactgccaatgacgcatgggatacgtcgtggcagtaaaagggcttaaatgccaacgacgcgtcccatacgttgttggcattttaattcttctctctgcagcggcagcatgtgccgccgctgcagagagtttctagcgatgacagcccctctgggcaacgagccgggggggctgtccctgcaggtacgatcaagcggcgcgcctttgcatttttagacatttagaagcctatatcttgttacagaattggaattacacaaaaattctaccatattttgaaagcttaggttgttctgaaaaaaacaatatattgttttcctgggtaaactaaaagtcccctcgaggaaaggcccctaaagtgaaacagtgcaaaacgttcaaaaactgtctggcaatacaagttccactttgaccaaaacggctggcagtaaaagggttaagcgatcgc

SEQ ID NO: 69 - DNA, 1803 bp, Artificial sequence
  piggyBac transposase #1 plasmid clone sequence
atcgatgccaccatgggcagctccctggacgatgagcacatcctgtccgccctgctgcagtctgacgatgagctggtgggcgaggacagcgattccgagatcagcgaccacgtgtccgaggacgatgtgcagtccgacacagaggaggccttcatcgacgaggtgcacgaggtgcagcccacctctagcggctccgagatcctggatgagcagaacgtgatcgagcagcctggctcctctctggcctctaataagatcctgaccctgccacagaggacaatccgcggcaagaacaagcactgctggtctaccagcaagtccacacggagaagccgggtgtccgccctgaaccacgtgcggtctcagagaggcccaaccaggatgtgccgcaatatctacgaccccctgctgtgctttaagctgttctttacagatgagatcatcagcgagatcgtgaagtggaccaacgccgagatctccctgaagaggcgcgagtctatgaccggcgccacattcagggacaccaatgaggatgagatctacgccttctttggcatcctggtcatgacagccgtgcggaaggacaaccacatgtccaccgacgatctgtttgatagatctctgagcatggtgtacgtgagcgtgatgagcagggaccgcttcgattttctgatccggtgcctgagaatggacgataagagcatccggcctacactgagagagaatgacgtgttcaccccagtgaggaagatctgggatctgtttatccaccagtgtatccagaactacacaccaggagcacacctgaccatcgacgagcagctgctgggcttccggggcagatgcccttttcgcatgtacatcccaaataagcccagcaagtatggcatcaagatcctgatgatgtgcgattccggcaccaagtacatgatcaacggcatgccatatctgggcaggggcacccagacaaatggcgtgcccctgggcgagtactatgtgaaggagctgagcaagcctgtgcacggctcctgccgcaacatcacatgtgacaattggttcaccagcatccccctggccaagaacctgctgcaggagccttataagctgaccatcgtgggcacagtgaggtccaacaagcgcgagatccccgaggtgctgaagaattccaggtctcgccctgtgggcacatctatgttctgctttgatggcccactgaccctggtgagctacaagcccaagcctgccaagatggtgtatctgctgagctcctgtgacgaggatgcctctatcaacgagagcaccggcaagccccagatggtcatgtactataatcagacaaagggcggcgtggacaccctggatcagatgtgcagcgtgatgacctgttcccggaagacaaatagatggcctatggccctgctgtacggcatgatcaacatcgcctgcatcaattctttcatcatctatagccacaacgtgtctagcaagggcgagaaggtgcagtccaggaagaagttcatgcgcaatctgtacatgtctctgacatcctcttttatgcggaagagactggaggcccccaccctgaagaggtatctgcgcgacaacatctccaatatcctgcctaacgaggtgccaggcacctccgacgattctacagaggagccagtgaccaagaagcggacctactgcacatattgtccctctaagatccggagaaaggccaacgccagctgcaagaagtgtaagaaagtgatctgtagagagcacaatatcgacatgtgccagagctgtttttgactcgag

SEQ ID NO: 70 - DNA, 1806 bp, Artificial sequence
  HypiggyBac transposase #2 plasmid clone sequence
ggatccgccaccatgggcagctccctggacgatgagcacatcctgtccgccctgctgcagtctgacgatgagctggtgggcgaggacagcgattccgaggtgagcgaccacgtgtccgaggacgatgtgcagagcgacacagaggaggccttcatcgatgaggtgcacgaggtgcagccaacctctagcggcagcgagatcctggatgagcagaacgtgatcgagcagcctggctcctctctggcctccaataagatcctgaccctgccacagaggacaatccgcggcaagaacaagcactgctggtctaccagcaagcctacacggagatcccgggtgtctgccctgaaccacgtgcggtcccagagaggcccaaccaggatgtgccgcaatatctacgaccccctgctgtgctttaagctgttctttacagatgagatcatcagcgagatcgtgaagtggaccaacgccgagatctccctgaagaggcgcgagagcatgacctccgccacattcagggacaccaatgaggatgagatctacgccttctttggcatcctggtcatgacagccgtgcggaaggacaaccacatgagcaccgacgatctgtttgatagatccctgtctatggtgtacgtgagcgtgatgagcagggaccgcttcgattttctgatccggtgcctgagaatggacgataagtccatccggcctacactgagagagaatgacgtgttcaccccagtgaggaagatctgggatctgtttatccaccagtgtatccagaactacacaccaggagcacacctgaccatcgacgagcagctgctgggcttccggggcagatgcccttttcgcgtgtacatcccaaataagccctctaagtatggcatcaagatcctgatgatgtgcgatagcggcaccaagtacatgatcaacggcatgccatatctgggcaggggcacccagacaaatggcgtgcccctgggcgagtactatgtgaaggagctgtccaagcctgtgcacggctcttgccgcaacatcacatgtgacaattggttcacctctatccccctggccaagaacctgctgcaggagccttataagctgaccatcgtgggcacagtgaggagcaacaagcgcgagatccccgaggtgctgaagaatagcaggtcccgccctgtgggcacatccatgttctgctttgatggcccactgaccctggtgtcttacaagcccaagcctgccaagatggtgtatctgctgagctcctgtgacgaggatgcctctatcaacgagagcaccggcaagccccagatggtcatgtactataatcagacaaagggcggcgtggacaccctggatcagatgtgcagcgtgatgacctgttcccggaagacaaatagatggcctatggccctgctgtacggcatgatcaacatcgcctgcatcaattctttcatcatctatagccacaacgtgtctagcaagggcgagaaggtgcagagcaggaagaagttcatgcgcaatctgtacatgggcctgacatcctcttttatgcggaagagactggaggcccccaccctgaagaggtatctgcgcgacaacatctccaatatcctgcctaaggaggtgccaggcacctccgacgattctacagaggagccagtgaccaagaagcggacctactgcacatattgtccctccaagatccggagaaaggcctctgccagctgcaagaagtgtaagaaagtgatctgtagagagcacaacatcgacatgtgccagtcttgtttttgataactcgag

SEQ ID NO: 71 - DNA, 1807 bp, Artificial sequence
  HypiggyBac transposase #3 plasmid clone sequence
aatcgatgccaccatgggatctagcctggacgatgaacacatcctctctgccctgctccagagcgacgacgagctggtcggcgaggatagcgacagcgaggtgtccgaccacgtgtcagaagatgatgtgcagagcgacaccgaggaagctttcatcgacgaggtccacgaggtgcaacccaccagcagcggaagcgagatcctggacgagcagaacgtgatcgagcagcctggctctagcctggccagcaaccggatcctgacactgcctcagcggacaatcagaggcaaaaacaagcactgctggtccaccagcaagagcacccggaggagcagagtgtctgctctgaacatcgtgcgctctcagagaggccccaccagaatgtgcagaaacatctacgaccccctgttgtgcttcaagctgtttttcaccgacgagattatctctgaaatcgtcaagtggacaaacgctgaaatcagcctgaagaggcgggaaagcatgacctccgccaccttcagagatacaaatgaggacgagatctacgccttcttcggcatcctggtgatgaccgccgtgcggaaagacaaccacatgagcaccgatgacctgtttgacagaagcctgagcatggtctacgtgtccgtgatgtcgagagacagattcgactttctgatccggtgcctgcggatggacgataagtctatcagacctaccctgagagaaaacgacgtgttcacccctgtgagaaagatctgggacctgttcatccaccagtgtatccagaactacacccctggcgcccacctgaccattgacgagcagctgctgggattcagaggcagatgcccttttcgggtgtacatccccaacaagcccagcaagtacggcatcaagatcctgatgatgtgtgacagcggtacaaagtacatgattaacggcatgccttatctgggcagaggaacccagaccaacggcgtgccacttggcgagtactacgtgaaggaactgtccaaaccagttcatggcagttgtagaaacatcacctgtgataactggttcacatctatccccctggctaagaacctgctgcaggagccttacaagctgacaatcgtgggcacagtgcgctctaacaagagggaaatccctgaggtgctgaaaaacagcagatcccggcctgtgggcacaagcatgttctgcttcgacggccctctgaccctggtgagctacaagcctaagccagccaagatggtgtatctgctgagcagctgcgacgaggatgccagcatcaacgagagcacaggcaagcctcaaatggtgatgtactacaaccagacaaagggaggcgtggacaccctcgatcagatgtgttctgtgatgacctgcagccggaagaccaatagatggcctatggccctgctgtacggcatgatcaacatcgcctgcatcaatagcttcatcatctacagccacaacgtgtcatccaaaggcgaaaaggtgcaaagcagaaagaagttcatgagaaacctgtacatgtccctgaccagcagcttcatgagaaagcggctggaagcccctacactgaagcggtatctgcgggataatatctctaatatcttacccaaagaggtgcccggaacaagcgatgatagcactgaggaacccgtcatgaagaaaagaacctactgcacctactgtcctagcaagatcagaagaaaagccaacgcctcttgcaagaagtgcaaaaaagtgatctgcagagagcacaatatcgacatgtgccagagctgcttttgataactcgag

SEQ ID NO: 72 - DNA, 1807 bp, Artificial sequence
  HypiggyBac transposase #4 plasmid clone sequence
aatcgatgccaccatgggcagcagcttggacgacgagcacatcctgtctgctctgctccagagcgacgacgagctggtgggcgaagattcagacagcgaggtgtcagaccacgtgtccgaggacgatgtgcagagcgataccgaggaagcctttattgacgaagtgcatgaggtgcaacctacatccagcggatctgaaatcctggacgagcagaacgtgatcgagcagcccggctctagcctggccagcaacagaaacctgaccctgccccagagaacaatcagaggcaagaacaagcactgctggtccacctctaaacctacccggcggagccgggccagcgccctgaacatcgtgcgatctcagcggggacctaccagaatgtgcagaaacatttacgacccactgctttgttttaagctgttcttcaccgacgaaatcatcagcgagatcgtgaaatggaccaacgccgagatcagccttaagagaagggaaagcatgacaagcgctacattcagagacaccaacgaggatgagatctacgccttcttcggcatcctggtgatgaccgccgtgcggaaggacaaccacatgagcacagatgacctgttcgatagaagcctgtctatggtctacgtgagcgtgatgagcagagatagattcgacttcctgatccggtgcctgcggatggacgataagagcatcagacctaccctgagagaaaacgacgtgttcaccccagtgcgcaaaatctgggacctgtttatccaccagtgcatccagaattacacccctggtgctcacctgaccatcgacgagcaactgctgggctttagaggaagatgccccttcagagtgtacatccctaacaaaccctctaagtacggcatcaagatcctgatgatgtgcgatagcggcaccaagtacatgatcaacggcatgccttatctgggcagaggcacccagaccaatggcgtgcccctgggagaatactacgtgaaggaactgagcaagcctgtgcacggcagctgtagaaatatcacctgcgacaactggttcacaagcatccccctggccaagaacctgctgcaggagccctacaagctgaccatcgttggcacagtgcggtctaacaagagagaaattcctgaggtgctgaagaacagccggagccgccccgtgggcaccagcatgttctgcttcgacggccctctgacactggttagctacaagccaaagcctgctaagatggtgtacctgctgagcagctgcgacgaggacgcctccatcaacgagtccacaggcaagccgcaaatggtgatgtactacaaccagacaaagggcggagtggacaccctggatcagatgtgtagcgtcatgacctgtagcagaaagaccaaccggtggcccatggccctgctgtacggaatgatcaacatcgcctgcatcaactcattcatcatctatagccacaacgtgtccagcaagggcgagaaggtgcagagccggaagaagttcatgagaaacctgtatatgggcctgacaagcagcttcatgcggaaaagactggaagcccctacactgaaaagatacctgcgggacaatatttctaatatcctgcctaaagaggtgcctggcacctccgatgacagcacagaggaacctgtcatgaagaaacggacctactgtacctactgccctagtaagatccgcagaaaggccagcgcctcttgcaagaagtgcaagaaagtgatctgcagagagcacaatatcgacatgtgccagtcctgtttctgataactcgag

SEQ ID NO: 73 - protein, 594 aa, Artificial sequence
  piggyBac#1 transposase amino acid sequence
MGSSLDDEHILSALLQSDDELVGEDSDSEISDHVSEDDVQSDTEEAFIDEVHEVQPTSSGSEILDEQNVIEQPGSSLASNKILTLPQRTIRGKNKHCWSTSKSTRRSRVSALNHVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVTKKRTYCTYCPSKIRRKANASCKKCKKVICREHNIDMCQSCF

SEQ ID NO: 74 - DNA, 36 bp, Artificial sequence
  piggyBac transposon minimal 5′ terminal inverted repeat
gcatgcgtcaattttacgcagactatctttctaggg

SEQ ID NO: 75 - DNA, 63 bp, Artificial sequence
  piggyBac transposon minimal 3′ terminal inverted repeat
ccctagaaagataatcatattgtgacgtacgttaaagataatcatgcgtaaaattgacgcatg

SEQ ID NO: 76 - DNA, 150 bp, Artificial sequence
  piggyBac minimal TIR transposon plasmid clone sequence
gcggccgcttaaccctagaaagataatcatattgtgacgtacgttaaagataatcatgcgtaaaattgacgcatgcctgcaggtacgatcaagcggcgcgccgcatgcgtcaattttacgcagactatctttctagggttaagcgatcgc

SEQ ID NO: 77 - DNA, 612 bp, Artificial sequence
  piggyBac transposon plasmid clone sequence
gcggccgcaacacgcagctagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttacctgcaggtacgatcaagcggcgcgcccatatctataacaagaaaatatatatataataagttatcacgtaagtagaacacgaaataacaatataattatcgtatgagttaaatcttaaaagtcacgtaaaagataatcatgcgtcattttgactcacgcggttgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagcgatcgc

SEQ ID NO: 78 - DNA, 1044 bp, Artificial sequence
  SB100 transposase #1 plasmid clone sequence (BamHI + XhoI)
ggatccgccaccatgggcaagtccaaggagatctctcaggacctgagaaagaggatcgtggatctgcacaagagcggcagctccctgggagcaatctccaagcgcctggcagtgcctcggtctagcgtgcagaccatcgtgcgcaagtacaagcaccacggcaccacacagccttcttatcggagcggccggagaagggtgctgagcccacgcgacgagcggacactggtgcgcaaggtgcagatcaacccccggaccacagccaaggatctggtgaagatgctggaggagaccggcacaaaggtgtccatctctaccgtgaagagagtgctgtacaggcacaacctgaagggccactccgccagaaagaagcctctgctgcagaataggcacaagaaggcaaggctgaggttcgcaaccgcacacggcgacaaggatcgcacattttggcggaacgtgctgtggtctgacgagaccaagatcgagctgttcggccacaatgatcacagatacgtgtggaggaagaagggcgaggcctgcaagcccaagaataccatccctacagtgaagcacggaggaggctccatcatgctgtggggatgttttgcagcaggaggaacaggcgccctgcacaagatcgacggcatcatggatgccgtgcagtatgtggacatcctgaagcagcacctgaagacctctgtgagaaagctgaagctgggcaggaagtgggtgttccagcacgacaacgatccaaagcacacaagcaaggtggtggccaagtggctgaaggacaataaggtgaaggtgctggagtggcccagccagtcccctgatctgaacccaatcgagaatctgtgggccgagctgaagaagagagtgagggcccggagacccaccaacctgacacagctgcaccagctgtgccaggaggagtgggccaagatccacccaaattactgtggcaagctggtggagggctatcccaagaggctgacccaggtgaagcagtttaagggcaacgccacaaagtattgataactcgag

SEQ ID NO: 79 - protein, 340 aa, Artificial sequence
  SB100 transposase amino acid sequence
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 80 - protein, 340 aa, Artificial sequence
  SB10 transposase amino acid sequence
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 81 - DNA, 226 bp, Artificial sequence
  SB transposon PT 5′ terminal inverted repeat
agttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 82 - DNA, 228 bp, Artificial sequence
  SB transposon PT 3′ terminal inverted repeat
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaact

SEQ ID NO: 83 - DNA, 227 bp, Artificial sequence
  SB transposon PT2 5′ terminal inverted repeat
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 84 - DNA, 228 bp, Artificial sequence
  SB transposon PT2 3′ terminal inverted repeat
ttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 85 - DNA, 227 bp, Artificial sequence; identical to SEQ ID NO: 83
  SB transposon PT3 terminal inverted repeat

SEQ ID NO: 86 - DNA, 227 bp, Artificial sequence
  SB transposon PT4 5′ terminal inverted repeat
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtgtacatacacgcg

SEQ ID NO: 87 - DNA, 229 bp, Artificial sequence
  SB transposon PT4 3′ terminal inverted repeat
gcgcgtgtatgtacacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactg

SEQ ID NO: 88 - DNA, 622 bp, Artificial sequence
  SB transposon PT3 plasmid clone sequence
gcggccgcatctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagcctgcaggtacgatcaagcggcgcgccctaaagccatgacatcattttctggaattttccaagctgtttaaaggcacagtcaacttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactgtatagcgatcgc

SEQ ID NO: 89 - DNA, 507 bp, Artificial sequence
  SB transposon PT4 plasmid clone sequence
gcggccgctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtgtacatacacgcgcctgcaggtacgatcaagcggcgcgccgcgcgtgtatgtacacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtatagcgatcgc

SEQ ID NO: 90 - DNA, 4830 bp, Artificial sequence
  06.01.1812_(A)pmaCMV(BGI)-MCS-IRES-ECFP plasmid sequence
actgcggccgccctgcaggtcaactagtgacgtcttaattaattgccggctggaacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattctggccaggatccgctagctctagagtcgacggtaccagtactaagcttgcctcgaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcaagcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgcatgccctaggtccggaaccggttggcgcgccatctggcagcgatcgccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaacgtacggaagttagagaaaaggcataagtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcatctcatgaaaattatgcaaattgagccagtcaggcagt

SEQ ID NO: 91 - DNA, 1830 bp, Artificial sequence
  06.01.1955_(A)pmk-MCS plasmid sequence
actgcggccgccctgcaggtcaactagtgacgtcttaattaattgccggctggaacgcgtttcgaacatcgattgaattctggccaagtggatccgctagctctagagtcgacggtaccaagcttgcctcgagccatggagatctgcatgccctaggtccggaaccggttggcgcgccatctggcagcgatcgccgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggcgacgacgggcgttccttgcgccgctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgactcatggcgatgcctgcttgccgaatatcatggtggaaaatggccgcttttctggattcatcgactgtggtcggctgggtgtggcggatcgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgataacgtacggaagttagagaaaaggcataagtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcatctcatgaaaattatgcaaattgagccagtcaggcagt

SEQ ID NO: 92 - DNA, 4689 bp, Artificial sequence
  PacI-hPGK-Luciferase-ires-EGFP-WPRE-AscI insert
ttaattaattgccggctggaacgcgttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctctctagtaccctcgcagacggacagcgccagggagcaatggcagcgcgccgaccgcgatgggctgtggccaatagcggctgctcagcagggcgcgcggagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagttcgaacatcgatgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagctcgaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatccgatatcatatgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagagtcgacggtaccagtactaagcttgcctcgagccatggagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcaagcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgcatgccctaggtccggaaccggttggcgcgcc

SEQ ID NO: 93 - DNA, 1083 bp, Artificial sequence
  SbfI-PuroR(R)-PacI insert
cctgcaggtcaactagttaagatacattgatgagtttggacaaaccacaactagaatgcagtgaaaaaaatgctttatttgtgaaatttgtgatgctattgctttatttgtaaccattataagctgcaataaacaagtttcaggcaccgggcttgcgggtcatgcaccaggtacgcggtccttcgggcacctcgacatcggcggtgacggtgaagccgagccgctcgtagaaggggaggttgcggggcgcggaggtttccagaaaggcgggcaccccggctctctcggctgcctccactccggggagaacgacggcggagcccagacccttgccctggtggtcgggcgagacaccgacggtagccaggaaccacgcgggctccttgggtcggtgcggtgccagaagtccctccatctgttgctgcgcggccagccgggaaccgctcaactcggccatgcgcgggccgatctcggcgaacaccgcccccgcttcgacgctctccggcgtggtccagaccgccaccgctgcgccgtcgtccgcgacccacaccttgccgatgtcgagcccgacgcgcgtgagaaacagctcttgcagctctgtgaccctctcgatgtggcggtcagggtctacggtgtggcgcgtggcggggtagtcggcgaacgcggcggcgagggtgcggacggctctaggaacatcgtcgcgggtggcgaggcgcaccgtgggcttgtactcagtcatagctttttgcaaaagcctatggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacttaattaa

SEQ ID NO: 94 - DNA, 1509 bp, Artificial sequence
  SbfI-HygroR(R)-PacI insert
cctgcaggtcaactagttaagatacattgatgagtttggacaaaccacaactagaatgcagtgaaaaaaatgctttatttgtgaaatttgtgatgctattgctttatttgtaaccattataagctgcaataaacaagttctattcctttgccctcggacgagtagaggggcgtctgtttccactatcggcgagaacttctacacagccatcggtccagacggctgcgcttctgcgggcgatttgtgttcgcccgacagtcccggctccacttcggacgattgcatcacatcgaccctgcgcccaagcagcatcatcgaaattgccgtccaccaagctctgatagagttggtcaagaccaatgcggagcatatacgcccggagccgaggcgatcctgcaagctctggatgtcgtctttcaaagtagcgcgtttgctgctccatacaagccaaccaaggacgccagaaaaagatgttagcgacctcgtattgggaatccccgaacatagcttcgctccaatcaatgaccgctgttatgcggccattatctgtcaggacattgttgctgccgaaatccgcgtggaccaagtggcgaacttcggggcaatcctcggcccaaagcatcagctcatcgagagcctgcgcgacactcgcgctgacggtatcatccatcacagtttgccaatgatacacatggggatcagcaatcgcgcaaatgaaatcacgccatgtagtgtattgaccgattccttgcggtccaaatgggccgaacccggaagtctgggaaagatcggcagcagcaatagcgtccatagcctccgcgacaggttgtagaacagcgggcagttcggtttcagggaggtcttgcaaagttacgccctgtgcgcggcgggagatgcaataggtcaggctctcgctgaactccccaatgtcaagcacttcgggaatcgggagcgcagccgatgcaaagtgccgataaacataacgatctttgtagaaaccatcggcgcagctatttacccgcaggacatatccacgccctcctacatcgaagctgaaagccctagattcctcgccctccgagagctgcatcaggtcgctaacgctgtcgaacttttcgatcagaaacttctcaacagaagtcgctgtgagttcaggctttttcatagctttttgcaaaagcctatggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacttaattaa

SEQ ID NO: 95 - DNA, 882 bp, Artificial sequence
  SbfI-BSD(R)-PacI insert
cctgcaggtcaactagttaagatacattgatgagtttggacaaaccacaactagaatgcagtgaaaaaaatgctttatttgtgaaatttgtgatgctattgctttatttgtaaccattataagctgcaataaacaagttttagccctcccacacataaccagagggcagcaattcacgaatcccaactgccgtcggctgtccatcactgtctttcactatggctttgataccaggatggaggtcaagaagcacctgtcggcatcttccgcagggacttaaaatgcccctgttctcatttccgatagcgactatacaagtcaggttgcctgctgccgccgccgccgcagttccaagcaccacaagttcagcacatggtcctcctgtaaaatgatatacattgacacctgtaaaaatacgaccatcactagagagagcagcagaagcgacagaataatcttcgctgataggaatactattgattgtagcagttgctctttcaatgagggtgctttcctcttgggacaatggtttagccatagctttttgcaaaagcctatggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacttaattaa

SEQ ID NO: 96 - DNA, 6391 bp, Artificial sequence
  SbfI-CMV(BGI)-gag/pol-AscI insert
cctgcaggcgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgccgggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaatagtctagaaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttctcgagccatggagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcaagcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttggcgcgcc

SEQ ID NO: 97 - DNA, 1282 bp, Artificial sequence
  SbfI-TRE2CuO-rev-AscI insert
cctgcaggttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtattaggcgtgtacggtgggcgcctataaaagcagagctcgtttagtgaaccgtcagatcgcctggagcaattccacatacaaacagaccagattgtctgtttgttacacttttgtcttataccaactttccgtaccacttcctaccctcgtaaattcgaaatcgatgccaccatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtaactcgagccatggagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcaagcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttggcgcgcc

SEQ ID NO: 98 - DNA, 2961 bp, Artificial sequence
  SbfI-TRE2CuO(BGI)-VSVG-AscI insert
cctgcaggttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtattaggcgtgtacggtgggcgcctataaaagcagagctcgtttagtgaaccgtcagatcgcctggagcaattccacatacaaacagaccagattgtctgtttgttacacttttgtcttataccaactttccgtaccacttcctaccctcgtaaattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaatcgatgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaatagctcgagccatggagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcaagcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttggcgcgcc

SEQ ID NO: 99 - DNA, 6439 bp, Artificial sequence
  SbfI-CAGGS(BGIC&R)-rtTA-CMV(BGI)-CymR-HygroR- AscI insert
cctgcagggacattgattattgacatgttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgttcgaaggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctcttgagcctctgctaaccatgttcatgccttcttctttttcctacagcttcgaacctgggcaacgtgctggttattgtgctgtctcatcattttggcaaaatcgatgccaccatgtccagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactcgaggatatcccatggagatctatggggacatcatgaagccccttgagcatctgacttctggctaataaaggaaatttattttcattgcaatagtgtgttggaattttttgtgtctctcactcggaaggacatatgggagccgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgatgccaccatgtctccaaagaggagaacccaggcagagagggcaatggagacacagggcaagctgatcgccgccgccctgggcgtgctgagggagaagggatacgcaggcttccgcatcgccgatgtgccaggagccgccggcgtgtcccggggcgcacagtctcaccacttccctaccaagctggagctgctgctggccacatttgagtggctgtatgagcagatcaccgagaggagccgcgccaggctggcaaagctgaagccagaggacgatgtgatccagcagatgctggacgatgccgccgagttctttctggacgatgactttagcatctccctggatctgatcgtggccgccgatagagaccccgccctgagggagggcatccagaggacagtggagagaaacaggttcgtggtggaggatatgtggctgggcgtgctggtgtctcgcggcctgagccgggatgacgcagaggacatcctgtggctgatctttaacagcgtgcggggcctggccgtgagatccctgtggcagaaggacaaggagcggttcgagcgcgtgcggaattccaccctggagatcgccagagagaggtacgccaagtttaagagatgataactcgagccatggagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcaagcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgcatgccctagtgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccataggcttttgcaaaaagctatgaaaaagcctgaactcacagcgacttctgttgagaagtttctgatcgaaaagttcgacagcgttagcgacctgatgcagctctcggagggcgaggaatctagggctttcagcttcgatgtaggagggcgtggatatgtcctgcgggtaaatagctgcgccgatggtttctacaaagatcgttatgtttatcggcactttgcatcggctgcgctcccgattcccgaagtgcttgacattggggagttcagcgagagcctgacctattgcatctcccgccgcgcacagggcgtaactttgcaagacctccctgaaaccgaactgcccgctgttctacaacctgtcgcggaggctatggacgctattgctgctgccgatctttcccagacttccgggttcggcccatttggaccgcaaggaatcggtcaatacactacatggcgtgatttcatttgcgcgattgctgatccccatgtgtatcattggcaaactgtgatggatgataccgtcagcgcgagtgtcgcgcaggctctcgatgagctgatgctttgggccgaggattgccccgaagttcgccacttggtccacgcggatttcggcagcaacaatgtcctgacagataatggccgcataacagcggtcattgattggagcgaagctatgttcggggattcccaatacgaggtcgctaacatctttttctggcgtccttggttggcttgtatggagcagcaaacgcgctactttgaaagacgacatccagagcttgcaggatcgcctcggctccgggcgtatatgctccgcattggtcttgaccaactctatcagagcttggtggacggcaatttcgatgatgctgcttgggcgcagggtcgatgtgatgcaatcgtccgaagtggagccgggactgtcgggcgaacacaaatcgcccgcagaagcgcagccgtctggaccgatggctgtgtagaagttctcgccgatagtggaaacagacgcccctctactcgtccgagggcaaaggaatagaacttgtttattgcagcttataatggttacaaataaagcaatagcatcacaaatttcacaaataaagcatttttttcactgcattctagttgtggtttgtccaaactcatcaatgtatcttaggcgcgcc

SEQ ID NO: 100 - DNA, 7091 bp, Artificial sequence
  SbfI-LVRSV-hPGK-Luciferase-ires-EGFP-PuroR-AscI insert
cctgcaggttaagtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagaatcgatattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagaattcttcgaaatggtaccaaggcctttttaaaagaaaaggggggattggggggtacagtgcaggggaaagaatagtagacataatagcaacagacatacaaactaaagaattacaaaaacaaattacaaaaattcaaaattttacgcgtggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgcggagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagctcgaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagatccgatatcatatgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcggccgcttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaggcgatcgcaacttgtttattgcagcttataatggttacaaataaagcaatagcatcacaaatttcacaaataaagcatttttttcactgcattctagttgtggtttgtccaaactcatcaatgtatcttacctagtgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggccataggcttttgcaaaaagctatgactgagtacaagcccacggtgcgcctcgccacccgcgacgatgttcctagagccgtccgcaccctcgccgccgcgttcgccgactaccccgccacgcgccacaccgtagaccctgaccgccacatcgagagggtcacagagctgcaagagctgtttctcacgcgcgtcgggctcgacatcggcaaggtgtgggtcgcggacgacggcgcagcggtggcggtctggaccacgccggagagcgtcgaagcgggggcggtgttcgccgagatcggcccgcgcatggccgagttgagcggttcccggctggccgcgcagcaacagatggagggacttctggcaccgcaccgacccaaggagcccgcgtggttcctggctaccgtcggtgtctcgcccgaccaccagggcaagggtctgggctccgccgtcgttctccccggagtggaggcagccgagagagccggggtgcccgcctttctggaaacctccgcgccccgcaacctccccttctacgagcggctcggcttcaccgtcaccgccgatgtcgaggtgcccgaaggaccgcgtacctggtgcatgacccgcaagcccggtgcctgaaacttgtttattgcagcttataatggttacaaataaagcaatagcatcacaaatttcacaaataaagcatttttttcactgcattctagttgtggtttgtccaaactcatcaatgtatcttaggcgcgcc

SEQ ID NO: 101 - DNA, 20 bp, Artificial sequence
  Primer S3F
attctgagtccaagctaggc

SEQ ID NO: 102 - DNA, 17 bp, Artificial sequence
  Primer S_IRES-R
gggccctcacattgcca

SEQ ID NO: 103 - DNA, 22 bp, Artificial sequence
  Primer TcBmN85-F
gcaacagctccctgaatcctgc

SEQ ID NO: 104 - DNA, 26 bp, Artificial sequence
  Primer TcBmN85-R
gagctgttgctcaccacctgttcgca

SEQ ID NO: 105 - DNA, 30 bp, Artificial sequence
  Primer TcBmA358-F
agagtgttcaagctgctgtgcgacgatctg

SEQ ID NO: 106 - DNA, 30 bp, Artificial sequence
  Primer TcBmA358-R
cacagcagcttgaacactctggcgttcaga

SEQ ID NO: 107 - DNA, 31 bp, Artificial sequence
  Primer TcBmI452-F
tcaatagcaagaaaagcaagctgaagctctg

SEQ ID NO: 108 - DNA, 34 bp, Artificial sequence
  Primer TcBmI452-R
cagcttgcttttcttgctattgattctgctgttc

SEQ ID NO: 109 - DNA, 39 bp, Artificial sequence
  Primer TcBmKE573-F
ctgagagagccgtgaagctgctgatgccttttgtgacca

SEQ ID NO: 110 - DNA, 39 bp, Artificial sequence
  Primer TcBmKE573-R
cagcagcttcacggctctctcagaaatctcggggaattc

SEQ ID NO: 111 - DNA, 30 bp, Artificial sequence
  Primer C_YabF321D-F
cgccaagaatgactacgtggtcaacctgga

SEQ ID NO: 112 - DNA, 31 bp, Artificial sequence
  Primer C_YabF321D-R
tgaccacgtagtcattcttggcgtccaccag

SEQ ID NO: 113 - DNA, 40 bp, Artificial sequence
  Primer C_YabSK-F
agcagacacgtgaacgtgaagggaagatacgtgaggtgtc

SEQ ID NO: 114 - DNA, 43 bp, Artificial sequence
  Primer C_YabSK-R
cttcccttcacgttcacgtgtctgctggaaggctcgcccagct

SEQ ID NO: 115 - DNA, 38 bp, Artificial sequence
  Primer C_optiHBm-F
gcgaatcgatgccaccatggacatcgagagacaggaag

SEQ ID NO: 116 - DNA, 36 bp, Artificial sequence
  Primer C_optiHBm#2-F
gcgaatcgatgccaccatggatatcgagcggcagga

SEQ ID NO: 117 - DNA, 33 bp, Artificial sequence
  Primer C_XtUP148T-F
cagaatcctctgaccagatacgccagagcacat

SEQ ID NO: 118 - DNA, 33 bp, Artificial sequence
  Primer C_XtUP148T-R
tctggcgtatctggtcagaggattctgggtcag

SEQ ID NO: 119 - DNA, 32 bp, Artificial sequence
  Primer C_XtUD359N-F
ctgtactgcctgaacacacctgcttgtggcac

SEQ ID NO: 120 - DNA, 31 bp, Artificial sequence
  Primer C_XtUD359N-R
agcaggtgtgttcaggcagtacagggcggta

SEQ ID NO: 121 - DNA, 37 bp, Artificial sequence
  Primer C_XtUA462H-F
aggaagaccagacactggtacaaaaaagtgggaatct

SEQ ID NO: 122 - DNA, 35 bp, Artificial sequence
  Primer C_XtUA462H-R
cacttttttgtaccagtgtctggtcttcctggtgg

SEQ ID NO: 123 - DNA, 73 bp, Artificial sequence
  Primer C_XtUF576R-R
gactctcgagttatcagtagtgcagctgggtgtggtagatctcgaaacagggcttccggcacaggccggggtt

SEQ ID NO: 124 - DNA, 29 bp, Artificial sequence
  Primer C_XtUS193K-F
cagtcctgaagatccccgtgttctctgct

SEQ ID NO: 125 - DNA, 31 bp, Artificial sequence
  Primer C_XtUS193K-R
aacacggggatcttcaggactgttgtggtgt

SEQ ID NO: 126 - DNA, 37 bp, Artificial sequence
  Primer C_XtU#1-F
gcgaatcgatgccaccatggccaagagattctacagc

SEQ ID NO: 127 - DNA, 36 bp, Artificial sequence
  Primer C_XtU#2-F
gcgaatcgatgccaccatggccaagcggttctacag

SEQ ID NO: 128 - DNA, 31 bp, Artificial sequence
  Primer C_SB100#2-F
agatcgacggcagcatggatgccgtgcagta

SEQ ID NO: 129 - DNA, 27 bp, Artificial sequence
  Primer C_SB100#2-R
catccatgctgccgtcgatcttgtgca

SEQ ID NO: 130 - DNA, 21 bp, Artificial sequence
  Primer WPRE-taqman-F
cctttccgggactttcgcttt

SEQ ID NO: 131 - DNA, 20 bp, Artificial sequence
  Primer WPRE-taqman-R
gcagaatccaggtggcaaca

SEQ ID NO: 132 - DNA, 22 bp, Artificial sequence
  Primer WPRE-Probe
actcatcgccgcctgccttgcc

(19) United States
(12) Patent Application Publication
(10) Pub. No.: US 2026/0234570 A1
(43) Pub. Date: Aug. 13, 2026

(54) MULTI-TRANSPOSON SYSTEM

(71) Applicant: Shenzhen Eureka Biotechnology Co., Limited, Shenzhen (CN)

(72) Inventors: Bofu XUE, Shenzhen (CN); Yinhui YANG, Shenzhen (CN); Jie LIU, Shenzhen (CN); Li CHEN, Shenzhen (CN)

(21) Appl. No.: 18/699,146

(22) PCT Filed: Oct. 15, 2021
(86) PCT No.: PCT/CN2021/124202

§ 371 Date: Apr. 5, 2024

(51) Int. Cl. C12N 7/00; C12N 9/16; C12N 15/85; C12N 15/90

(52) U.S. Cl. CPC C12N 7/00; C12N 9/16; C12N 15/85; C12N 15/907; C12N 2740/15052; C12N 2800/107; C12N 2800/90

(57) ABSTRACT

Provided in the present disclosure is a method for integrating one or more exogenous nucleotide sequences into a mammalian host cell genome. The method comprises integrating the one or more exogenous nucleotide sequences into the mammalian host cell genome using at least two transposon systems.

### SEQUENCE LISTING

The instant application contains a Sequence Listing which has been submitted electronically in ASCII format and is hereby incorporated by reference in its entirety. Said ASCII copy, created on Jun. 27, 2025, is named 52392-0007US1_ST25.txt.

### TECHNICAL FIELD

The present disclosure relates to a method for integrating exogenous nucleotide sequences into a host cell genome, and in particular, to a method for integrating exogenous nucleotide sequences into a mammalian host cell using a multi-transposon system.

### BACKGROUND ART

Transposon refers to a DNA sequence that can change its position within a genome. Transposon can create or reverse mutations and change the size of a cell genome. DNA transposon can translocate from one DNA site to another in a simple cut-and-paste manner under the action of an expressed transposase. Transposition is a precise process in which defined DNA fragments (usually direct repeats (DRs) and invert repeats (IRs) attached thereto at the two terminals of a transposon, and an insert sequence (IS) in the middle) are excised from one DNA molecule and moved to another site in the same or different DNA molecule or genome.

At present, methods for inserting genes of interest into host cell genomes using transposon systems have been disclosed. However, there is an upper limit on the number of inserted copies for most transposon systems when target genes are inserted. Especially when the insertion of multiple target genes is involved, the existing methods fail to insert multiple target genes at a high copy number, and also fail to effectively adjust the ratio of the copy number of multiple target genes inserted.

### Technical Problem

In one aspect, the present disclosure provides a method for integrating one or more exogenous nucleotide sequences into a mammalian host cell genome, and the method comprises integrating the one or more exogenous nucleotide sequences into the mammalian host cell genome using at least two transposon systems.

### SUMMARY

In an embodiment, in the method of the present disclosure for integrating one or more exogenous nucleotide sequences into a mammalian host cell genome, the at least two transposon systems include: a Tol1 transposon system, a Tol2 transposon system, a Frog Prince transposon system, a Minos transposon system, a Hsmar1 transposon system, a Helraiser transposon system, a ZB transposon system, an Intruder transposon system, a SPINON transposon system, a TcBuster transposon system, a Passport transposon system, a Yabusame-1 transposon system, a Uribo2 transposon system, a PiggyBac (PB) transposon system, a Sleeping Beauty (SB) transposon system, and various variants or derivatives of the above-mentioned transposon systems.

In another embodiment, in the method of the present disclosure for integrating one or more exogenous nucleotide sequences into a mammalian host cell genome, the at least two transposon systems include: a Tol1 transposon system, a Tol2 transposon system, a ZB transposon system, an Intruder transposon system, a TcBuster transposon system, a Yabusame-1 transposon system, a Uribo2 transposon system, a PB transposon system and a SB transposon system, and various variants or derivatives of the above-mentioned transposon systems.

In another embodiment, the one or more exogenous nucleotide sequences are integrated into the mammalian host cell genome simultaneously or successively using the at least two transposon systems.

In another aspect, the present disclosure provides a mammalian cell that comprises one or more exogenous nucleotide sequences integrated in a mammalian cell genome and is obtained using the method of the present disclosure as described above.

In another aspect, the present disclosure provides a mammalian cell comprising at least two transposons integrated in the genome of the mammalian cell.

In an embodiment, in the genome of the mammalian cell, the sequences of the at least two transposons do not overlap with each other.

In an embodiment, in the mammalian cell of the present disclosure, the at least two transposons include: a Tol1 transposon, a Tol2 transposon, a Frog Prince transposon, a Minos transposon, a Hsmar1 transposon, a Helraiser transposon, a ZB transposon, an Intruder transposon, a SPINON transposon, a TcBuster transposon, a Passport transposon, a Yabusame-1 transposon, a Uribo2 transposon, a PiggyBac (PB) transposon, a Sleeping Beauty (SB) transposon, and various variants or derivatives of the above-mentioned transposons.

In another embodiment, in the mammalian cell of the present disclosure, the at least two transposons include: a Tol1 transposon, a Tol2 transposon, a ZB transposon, an Intruder transposon, a TcBuster transposon, a Yabusame-1 transposon, a Uribo2 transposon, a PB transposon and a SB transposon, and various variants or derivatives of the above-mentioned transposons.

In another aspect, the present disclosure provides a method for constructing a lentivirus producing cell line, wherein the method comprises integrating the sequences of gag, pol and rev genes of a lentivirus, a sequence encoding a viral envelope protein, and a viral genome transcription cassette sequence carrying a nucleic acid fragment of interest into a host cell genome using at least two transposon systems.

In an embodiment, in the method of the present disclosure for constructing a lentivirus producing cell line, the at least two transposon systems include: a Tol1 transposon system, a Tol2 transposon system, a Frog Prince transposon system, a Minos transposon system, a Hsmar1 transposon system, a Helraiser transposon system, a ZB transposon system, an Intruder transposon system, a SPINON transposon system, a TcBuster transposon system, a Passport transposon system, a Yabusame-1 transposon system, a Uribo2 transposon system, a PiggyBac (PB) transposon system, a Sleeping Beauty (SB) transposon system, and various variants or derivatives of the above-mentioned transposon systems.

In another embodiment, in the method of the present disclosure for constructing a lentivirus producing cell line, the at least two transposon systems include: a Tol1 transposon system, a Tol2 transposon system, a ZB transposon system, an Intruder transposon system, a TcBuster transposon system, a Yabusame-1 transposon system, a Uribo2 transposon system, a PB transposon system and a SB transposon system, and various variants or derivatives of the above-mentioned transposon systems.

In another aspect, the present disclosure provides a lentivirus producing cell line, wherein the lentivirus producing cell line comprises at least two transposons integrated in the genome thereof.

In an embodiment, in the lentivirus producing cell line of the present disclosure, the at least two transposons include: a Tol1 transposon, a Tol2 transposon, a Frog Prince transposon, a Minos transposon, a Hsmar1 transposon, a Helraiser transposon, a ZB transposon, an Intruder transposon, a SPINON transposon, a TcBuster transposon, a Passport transposon, a Yabusame-1 transposon, a Uribo2 transposon, a PiggyBac (PB) transposon, a Sleeping Beauty (SB) transposon, and various variants or derivatives of the above-mentioned transposons.

In another embodiment, in the lentivirus producing cell line of the present disclosure, the at least two transposons comprise: a Tol1 transposon, a Tol2 transposon, a ZB transposon, an Intruder transposon, a TcBuster transposon, a Yabusame-1 transposon, a Uribo2 transposon, a PB transposon and a SB transposon, and various variants or derivatives of the above-mentioned transposons.

### DETAILED DESCRIPTION OF EMBODIMENTS

As used herein, “integration of one or more exogenous nucleotide sequences into a host cell genome simultaneously or successively using at least two transposon systems” includes, for example, simultaneously or successively using two or more transposon systems of the present disclosure to integrate the same exogenous nucleotide sequence into a host cell genome, and also includes simultaneously or successively using two or more transposon systems of the present disclosure to integrate two or more exogenous nucleotide sequences into a host cell genome.

As used herein, “one or more exogenous nucleotide sequences that are integrated in the genome of the cell and flanked by the recognition sequences of the at least two transposon systems” means that one or more exogenous nucleotide sequences are integrated in a host cell genome and that the recognition sequences of at least two types of the transposon systems of the present disclosure are simultaneously present on both sides of various copies of the one or more exogenous nucleotide sequences. As can be appreciated, for example, when two or more transposon systems of the present disclosure are simultaneously or successively used to integrate the same exogenous nucleotide sequence into a host cell genome, various copies of the exogenous nucleotide sequence integrated in the host cell genome will be flanked by the recognition sequences of corresponding different types of transposon systems; when two or more transposon systems of the present disclosure are simultaneously or successively used to integrate two or more exogenous nucleotide sequences into a host cell genome, various copies of the two or more exogenous nucleotide sequences integrated in the host cell genome will also be flanked by the recognition sequences of corresponding different types of transposon systems.

As used herein, the term “transposon” or “transposable element” refers to a polynucleotide that can be cleaved from a first polynucleotide by the action of a trans-acting transposase and integrated into a second position of the same polynucleotide or into a second polynucleotide. The transposon comprises a first transposon terminal and a second transposon terminal. The first transposon terminal and the second transposon terminal are polynucleotide sequences that are recognized and transposed by a transposase, and may be referred to herein as recognition sequences of a transposon system. The transposon typically further comprises a polynucleotide sequence of interest located between the two transposon terminals such that the polynucleotide sequence of interest is transposed together with the two transposon terminals under the action of the transposase. As used herein, the term “transposon terminal” or “recognition sequence of transposon system” refers to a cis-acting nucleotide sequence sufficient to be recognized and transposed by a transposase. A pair of transposon terminals typically comprise pairs of perfect or imperfect repeats such that the corresponding repeats in the pairs of elements in the two different transposon terminals are inversely complementary to each other. These are referred to as inverted terminal repeats (ITRs) or terminal inverted repeats (TIRs). The transposon terminal may or may not comprise additional sequences adjacent to ITR for promoting or enhancing transposition. As used herein, the “transposon system” comprises the “transposon” or “transposable element” as described above and a corresponding transposase that can recognize and move the “transposon” or “transposable element” in a trans-acting manner. As used herein, “the sequences of the at least two transposons do not overlap with each other” means that, for example, when two transposons are used, assuming that the recognition sequences at the two terminals of the first transposon are L1 and R1, respectively, and the recognition sequences at the two terminals of the second transposon are L2 and R2, respectively, the following arrangements are not present in the mammalian cell genome, for example: L1-L2-R2-R1, L1-L2-R1-R2, L1-R2-L2-R1, L1-R2-R1-L2, R1-L2-R2-L1, R1-L2-L1-R2, R1-R2-L2-L1, R1-R2-L1-L2, L2-L1-R1-R2, L2-L1-R2-R1, L2-R1-L1-R2, L2-R1-R2-L1, R2-L1-R1-L2, R2-L1-L2-R1, R2-R1-L1-L2, and R2-R1-L2-L1.

In the present disclosure, transposon systems that may be used include: a Tol1 transposon system, a Tol2 transposon system, a Frog Prince transposon system, a Minos transposon system, a Hsmar1 transposon system, a Helraiser transposon system, a ZB transposon system, an Intruder transposon system, a SPINON transposon system, a TcBuster transposon system, a Passport transposon system, a Yabusame-1 transposon system, a Uribo2 transposon system, a PiggyBac (PB) transposon system, a Sleeping Beauty (SB) transposon system, and various variants or derivatives of the above-mentioned transposon systems.

### Tol1 Transposon System

The description of the Tol1 transposon system can be found in, for example, international application WO 2008072540 (which is incorporated herein by reference). Herein, the term “Tol1 transposon system” may include a Tol1 transposon system comprising a corresponding transposase and various variants thereof as well as a corresponding transposon and various variants thereof.

### Tol2 Transposon System

The description of the Tol2 transposon system can be found in, for example, Ni, J. et al. (2016): Active recombinant Tol2 transposase for gene transfer and gene discovery applications. In Mob DNA7, p. 6 (which is incorporated herein by reference), and Kawakami K, Shima A. (1999): Identification of the Tol2 transposase of the medaka fish Oryzias latipes that catalyzes excision of a nonautonomous Tol2 element in zebrafish Danio rerio. In Gene. 1999 Nov. 15; 240 (1): 239-44 (which is incorporated herein by reference). For the optimization of TIR sequences, reference may be made to, for example, Urasaki, A. et al. (2006): Functional dissection of the Tol2 transposable element identified the minimal cis-sequence and a highly repetitive sequence in the subterminal region essential for transposition. In Genetics 174 (2), pp. 639-649 (which is incorporated herein by reference). The minimal TIR sequence is T2AL200R150G (GenBank accession number AB262452). Herein, the term “Tol2 transposon system” may include a Tol2 transposon system comprising a corresponding transposase and various variants thereof as well as a corresponding transposon and various variants thereof.

### Frog Prince (FP) Transposon System

The description of the Frog Prince transposon system can be found in, for example, international application WO 2003100070 (which is incorporated herein by reference) and Miskey C. et al. The Frog Prince: a reconstructed transposon from Rana pipiens with high transpositional activity in vertebrate cells. Nucleic Acids Res. 2003; 31 (23): 6873-6881 (which is incorporated herein by reference). Herein, the term “Frog Prince transposon system” may include a Frog Prince transposon system comprising a corresponding transposase and various variants thereof as well as a corresponding transposon and various variants thereof.

### Minos Transposon System

The description of the Minos transposon system can be found in, for example, Metaxakis, Athanasios et al. (2005): Minos as a genetic and genomic tool in Drosophila melanogaster. In Genetics 171 (2), pp. 571-581 (which is incorporated herein by reference) and Franz, G. et al. (1991): Minos, a new transposable element from Drosophila hydei, is a member of the Tc1-like family of transposons. In Nucleic acids research 19 (23), p. 6646 (which is incorporated herein by reference). Herein, the term “Minos transposon system” may include a Minos transposon system comprising a corresponding transposase and various variants thereof as well as a corresponding transposon and various variants thereof.

### Hsmar1 Transposon System

The description of the Hsmar1 transposon system can be found in, for example, international application WO 2006108525 (which is incorporated herein by reference), and Miskey, Csaba et al. (2007): The ancient mariner sails again: transposition of the human Hsmar1 element by a reconstructed transposase and activities of the SETMAR protein on transposon terminals. In Molecular and cellular biology 27 (12), pp. 4589-4600 (which is incorporated herein by reference). Herein, the term “Hsmar1 transposon system” may include an Hsmar1 transposon system comprising a corresponding transposase and various variants thereof as well as a corresponding transposon and various variants thereof.

### Helraiser Transposon System

Helraiser is an active Helitron transposon reconstructed using a bioinformatics method. The description of the Helraiser transposon system can be found in, for example, Grabundzija, Ivana et al. (2016): A Helitron transposon reconstructed from bats reveals a novel mechanism of genome shuffling in eukaryotes. In Nature communications 7, p. 10716 (which is incorporated herein by reference), Grabundzija, Ivana et al. (2018): Helraiser intermediates provide insight into the mechanism of eukaryotic replicative transposition. In Nature communications 9 (1), p. 1278 (which is incorporated herein by reference), and patent application US20190323037 (which is incorporated herein by reference). Herein, the term “Helraiser transposon system” may include a Helraiser transposon system comprising a corresponding transposase and various variants thereof as well as a corresponding transposon and various variants thereof.

### ZB Transposon System

The description of the ZB transposon system can be found in, for example, Shen, Dan et al. (2021): A native, highly active Tc1/mariner transposon from zebrafish (ZB) offers an efficient genetic manipulation tool for vertebrates. In Nucleic acids research 49 (4), pp. 2126-2140 (which is incorporated herein by reference), and CN 105018523B (which is incorporated herein by reference). Herein, the term “ZB transposon system” may include a ZB transposon system comprising a corresponding transposase and various variants thereof as well as a corresponding transposon and various variants thereof.

### Intruder (IT) Transposon System

The description of the Intruder transposon system can be found in, for example, Gao, Bo et al. (2020): Intruder (DD38E), a recently evolved sibling family of DD34E/Tc1 transposons in animals. In Mobile DNA 11 (1), p. 32 (which is incorporated herein by reference). Herein, the term “Intruder transposon system” may include an Intruder transposon system comprising a corresponding transposase and various variants thereof as well as a corresponding transposon and various variants thereof.

### SPINON Transposon System

The description of the SPINON transposon system can be found in, for example, Gilbert, C. et al. (2012): Rampant Horizontal Transfer of SPIN Transposons in Squamate Reptiles. In Molecular biology and evolution 29 (2), pp. 503-515 (which is incorporated herein by reference), and Li, Xianghong et al. (2013): A resurrected mammalian hAT transposable element and a closely related insect element are highly active in human cell culture. In Proceedings of the National Academy of Sciences of the United States of America 110 (6), E478-87 (which is incorporated herein by reference). Herein, the term “SPINON transposon system” may include a SPINON transposon system comprising a corresponding transposase and various variants thereof as well as a corresponding transposon and various variants thereof.

### TcBuster Transposon System

The description of the TcBuster transposon system can be found in, for example, Woodard, Lauren E. et al. (2012): Comparative analysis of the recently discovered hAT transposon TcBuster in human cells. In PloS one 7 (11), e42666 (which is incorporated herein by reference), Li, Xianghong et al. (2013): A resurrected mammalian hAT transposable element and a closely related insect element are highly active in human cell culture. In Proceedings of the National Academy of Sciences of the United States of America 110 (6), E478-87 (which is incorporated herein by reference), and US20180216087 (which is incorporated herein by reference) and CN 108728477A (which is incorporated herein by reference). The sequence of TcBusterCO (original TcBuster) transposase can be found in Li, Xianghong et al. (2013) above. The sequences of a variety of TcBuster transposase variants having enhanced activity are described in US 20180216087. US20180216087 and CN 108728477A describe a variety of 5′ TIR and 3′ TIR sequence variants of the TcBuster transposon system, respectively. The various 5′ TIRs and 3′ TIRs described above can be used in combination. Herein, the term “TcBuster transposon system” may include a TcBuster transposon system comprising a corresponding transposase and various variants thereof as well as a corresponding transposon and various variants thereof.

### Passport Transposon System

The description of the Passport transposon system can be found in, for example, Clark, Karl J. et al. (2009): Passport, a native Tc1 transposon from flatfish, is functionally active in vertebrate cells. In Nucleic acids research 37 (4), pp. 1239-1247 (which is incorporated herein by reference), and WO 2010008564 (which is incorporated herein by reference). Herein, the term “Passport transposon system” may include a Passport transposon system comprising a corresponding transposase and various variants thereof as well as a corresponding transposon and various variants thereof.

### Yabusame-1 Transposon System and Uribo2 Transposon System

Two new transposon-transposase systems are disclosed, one derived from Bombyx mori (Yabusame-1 transposon system) and the other from Xenopus tropicalis (Uribo2 transposon system), each of which comprises sequences that act as transposon terminals and are used in combination with a transposase that recognizes and acts on them. For the description of the above transposon-transposase systems, the corresponding transposases, and the sequence information of TIRs at the two terminals of the transposons, reference may be made to, for example, Hottentot Q P et al., Targeted Locus Amplification and Next-Generation Sequencing. In Genotyping: Methods and Protocols. White S J, Cantsilieris S, eds: 185-196. (New York, NY: Springer): 2017. pp. 185-196; Hikosaka, Akira et al. (2007): Evolution of the Xenopus piggyBac transposon family TxpB: domesticated and untamed strategies of transposon subfamilies. In Molecular biology and evolution 24 (12), pp. 2648-2656; GenBank accession number BAD11135.1; GenBank accession number BAF82022; WO 2017062668; WO 2019028273; U.S. Pat. No. 9,428,767B2; and U.S. Pat. No. 10,041,077B2 (all of which are incorporated herein by reference). Herein, the term “Yabusame-1 transposon system” may include a Yabusame-1 transposon system comprising a corresponding transposase and various variants thereof as well as a corresponding transposon and various variants thereof. Herein, the term “Uribo2 transposon system” may include a Uribo2 transposon system comprising a corresponding transposase and various variants thereof as well as a corresponding transposon and various variants thereof.

### PiggyBac Transposon System

The “PiggyBac (PB) transposon system” derived from Trichoplusia ni consists of a PB transposase and a transposon, which enable efficient transposition between a vector and a chromosome via a cut-and-paste mechanism. During transposition, the PB transposase recognizes transposon-specific inverted terminal repeats (ITRs) located at the two terminals of a transposon vector, and effectively moves an insert sequence (from the original site) between 5′ ITR and 3′ ITR and effectively integrates the insert sequence into the chromosomal TTAA site. The powerful activity of the PiggyBac transposon system allows the insert sequence of interest between the two ITRs in the PB transposon vector to be easily moved into a target genome. Wild-type and various variants (such as ePiggyBac) of transposases and transposons in the PB transposon system are known in the art. For the information of the PiggyBac transposon system, reference may be made to, e.g., patent documents U.S. Pat. No. 9,428,767B2, U.S. Pat. No. 10,041,077B2, U.S. Pat. No. 6,218,185B1, U.S. Pat. No. 6,551,825B1, U.S. Pat. No. 7,105,343B1, U.S. Pat. No. 6,962,810B2, U.S. Pat. No. 8,592,211B2, WO 2006122442, WO 2010085699, WO 2010099296, WO 2010099301, WO 2012074758, U.S. Pat. No. 8,592,211B2, etc., and non-patent documents: Lacoste, A. et al. (2009). “An efficient and reversible transposable system for gene delivery and lineage-specific differentiation in human embryonic stem cells.” Cell Stem Cell 5 (3): 332-342; Yusa, K. et al. (2011). “A hyperactive piggyBac transposase for mammalian applications.” Proc Natl Acad Sci USA 108 (4): 1531-1536; Meir, Yaa-Jyuhn J. et al. (2011): Genome-wide target profiling of piggyBac and Tol2 in HEK 293: pros and cons for gene discovery and gene therapy. In BMC biotechnology 11, p. 28; Troyanovsky, Boris et al. (2016): The Functionality of Minimal PiggyBac Transposons in Mammalian Cells. In Molecular therapy. Nucleic acids 5 (10), e369; Wen, Wen et al. (2020): An efficient Screening System in Yeast to Select a Hyperactive piggyBac Transposase for Mammalian Applications. In International journal of molecular sciences 21 (9) (the above-mentioned documents are incorporated herein by reference) and GenBank accession number ABC67521. Herein, the term “PiggyBac (PB) transposon system” may include a PB transposon system comprising a corresponding transposase and various variants thereof as well as a corresponding transposon and various variants thereof.

### Sleeping Beauty Transposon System

The Sleeping Beauty (SB) transposon system consists of a SB transposase and a transposon, which enable the insertion of a specific DNA insert sequence into the genome of a vertebrate. The SB transposase can insert a transposon into a TA dinucleotide base pair in a receptor DNA sequence. The insertion site may be located elsewhere in the same DNA molecule (or chromosome) or in another DNA molecule (or chromosome). The SB transposon consists of an insert sequence of interest and IR/DR sequences (inverted repeats (IRs) themselves containing short direct repeats (DRs)) located at the two terminals of the insert sequence for being recognized by a SB transposase. The transposase may be encoded within the transposon, or the transposase may be provided by another source. Wild-type and various variants of transposases, IR/DR sequences and transposons in the SB transposon system are known in the art. For the description and sequence information of the SB transposase and various variants thereof (e.g., SB1 to SB10, SB11, SB17, SB100X, and SB130X), reference may be made to, for example, Ivics, Zoltán et al. (1997): Molecular Reconstruction of Sleeping Beauty, a Tc1-like Transposon from Fish, and Its Transposition in Human Cells. In Cell 91 (4), pp. 501-510; Baus, James et al. (2005): Hyperactive transposase mutants of the Sleeping Beauty transposon. In Molecular therapy: the journal of the American Society of Gene Therapy 12 (6), pp. 1148-1156; Mátés, Lajos et al. (2009): Molecular evolution of a novel hyperactive Sleeping Beauty transposase enables robust stable gene transfer in vertebrates. In Nature genetics 41 (6), pp. 753-761; Voigt, Franka et al. (2016): Sleeping Beauty transposase structure allows rational design of hyperactive variants for genetic engineering. In Nature communications 7, p. 11126; Kesselring, Lisa et al. (2020): A single amino acid switch converts the Sleeping Beauty transposase into an efficient unidirectional excisionase with utility in stem cell reprogramming. In Nucleic acids research 48 (1), pp. 316-331; Querques, Irma et al. (2019): A highly soluble Sleeping Beauty transposase improves control of gene insertion. In Nature biotechnology 37 (12), pp. 1502-1512; WO 9840510A1; WO 2003089618A2; WO 2009003671A2; WO 2017046259A1; WO 2017158029A1 (the above-mentioned documents are incorporated herein by reference). For the description and related sequence information of the TIRs of the SB transposon and transposon vectors (e.g., pT1, pT2, pT3, and pT4), reference may further be made to, for example, Yant, Stephen R. et al. (2004): Mutational analysis of the N-terminal DNA-binding domain of sleeping beauty transposase: critical residues for DNA binding and hyperactivity in mammalian cells. In Molecular and cellular biology 24 (20), pp. 9239-9247; Cui, Zongbin et al. (2002): Structure-Function Analysis of the Inverted Terminal Repeats of the Sleeping Beauty Transposon. In Journal of molecular biology 318 (5), pp. 1221-1235; Izsvák, Zsuzsanna et al. (2002): Involvement of a bifunctional, paired-like DNA-binding domain and a transpositional enhancer in Sleeping Beauty transposition. In The Journal of biological chemistry 277 (37), pp. 34581-34588; Wang, Yongming et al. (2017): Regulated complex assembly safeguards the fidelity of Sleeping Beauty transposition. In Nucleic acids research 45 (1), pp. 311-326; and Zayed, Hatem et al. (2004): Development of hyperactive sleeping beauty transposon vectors by mutational analysis. In Molecular therapy: the journal of the American Society of Gene Therapy 9 (2), pp. 292-304. Herein, the term “Sleeping Beauty (SB) transposon system” may include a SB transposon system comprising a corresponding transposase and various variants thereof as well as a corresponding transposon and various variants thereof.

As used herein, the “exogenous nucleotide sequence” or “nucleotide sequence of interest” that can be integrated into a host cell genome may be, for example, a gene, such as a nucleic acid sequence encoding a polypeptide or protein; a nucleotide sequence that can be transcribed into a functional ribonucleic acid (RNA), such as a sequence encoding a small interfering RNA (siRNA), a long non-coding RNA (LncRNA), a guide RNA (gRNA) of a CRISPR gene editing system, a transfer RNA (tRNA), a ribosomal RNA (rRNA) or other functional ribonucleic acids; an element that regulates gene expression, e.g., a promoter, an enhancer, an intron, a terminator, a translation initiation signal, a polyadenylation signal, a virus-derived replication element, a RNA processing and export element, a post transcriptional responsive element, a matrix attachment element, an insulator, and an element that affects chromatin structure; other functional nucleic acid sequences, such as homologous recombination sequences, DNA or RNA sequences that can bind to proteins, and nucleotide sequences that can bind to other nucleic acid fragments (such as primers or probes) for detection; any of nucleic acid sequences in nature or artificial nucleotide sequences; and may also be a combination of one or more of the above nucleotide sequences.

Transposons and transposases may enter cells to accomplish the transposition function through a variety of ways, for example, delivery into cells by means of plasmid transient transfection, viral vector transduction, transfection of RNA encoding a transposase, or transfection of a transposase protein. The present disclosure takes the plasmid transient transfection as an example, but other methods for delivering a transposon system are known in the art, and changes in the delivery methods do not affect the spirit and principles of the present application and shall fall within the protection scope of the claims.

### EXAMPLES

The following examples are provided to illustrate the technical solutions of the present application and should not be considered as limiting the scope and spirit of the present application.

### Example 1: Plasmid Construction

Molecular cloning techniques, such as PCR amplification of DNA fragments, restriction enzyme digestion of DNA fragments, condensation recovery of DNA fragments, ligation of two or more DNA fragments using T4 DNA ligase, transformation of ligation products in competent cells, and plasmid extraction and identification, used in the following examples are known in the art. Reagents involved in the following examples are as follows: PCR enzyme (Thermo, F-530S); restriction enzymes (NEB); T4 DNA ligase (Invitrogen, 15224041); DNA fragment condensation recovery kit (Omega, D2500-02); plasmid miniprep kit (TIANGEN, DP 105-03); competent cells (EPI400, Lucigen Inc., C400CH10). The nucleic acid sequences denoted by “GenScript synthesis” in Table 1 below were synthesized by Genscript Biotech Corporation and used to construct the plasmids used in the present disclosure. The primers for plasmid construction, transposase mutation and qPCR detection in Table 2 below were synthesized by General Biosystems (Anhui) Co., Ltd. The plasmids were sequenced and identified by GUANGZHOU IGE BIOTECHNOLOGY LTD. Table 3 below lists plasmid numbers, plasmid names, nucleic acid serial numbers of inserts, restriction enzyme sites for insertion, and numbers of inserted plasmid vectors used in the present disclosure. The sequence information of the functional elements employed in the respective plasmids and examples demonstrating the utility of the present disclosure referred to in the following examples are only as examples for implementing the present disclosure, and should not be considered as limiting the scope of protection of the present application. Those skilled in the art would have been able to expect to replace the sequences of the functional elements on the plasmids used in the following examples with other sequences having similar biological functions to achieve the technical effects of the present disclosure, the above-mentioned sequences including, but not limited to, backbone sequences (such as replication origin and resistant genes), restriction endonuclease sites, transposon repeats, response element sequences of inducible expression systems, insulator sequences, promoter sequences, intron sequences, PolyA sequences, various codon-optimized gene sequences, mutants of the above functional element sequences and gene sequences, and the cloning locations, cloning sequences and cloning directions of the above functional element sequences and gene sequences. Specific methods for constructing plasmids are described below:

1. Construction of transposase plasmid: the synthesized sequences SEQ ID NO: 1, SEQ ID NO: 6, SEQ ID NO: 11, SEQ ID NO: 15, SEQ ID NO: 19, SEQ ID NO: 23, SEQ ID NO: 28, SEQ ID NO: 32, SEQ ID NO: 36, SEQ ID NO: 41, SEQ ID NO: 49, SEQ ID NO: 54, SEQ ID NO: 55, SEQ ID NO: 60, SEQ ID NO: 61, SEQ ID NO: 69, SEQ ID NO: 72 and SEQ ID NO: 71 were digested with restriction enzymes ClaI and XhoI, respectively, and ligated to the restriction sites ClaI and XhoI of plasmid 06.01.1812 (SEQ ID NO: 90), thereby constructing plasmids 06.01.1494, 06.01.1504, 06.01.1527, 06.01.1535, 06.01.1538, 06.01.1579, 06.01.1573, 06.01.1582, 06.01.1596, 06.01.1614, 06.01.1679, 06.01.1687, 06.01.1740, 06.01.1770, 06.01.1790, 06.01.1581, 06.01.1892 and 06.01.1903, respectively. The synthesized sequences SEQ ID NO: 70 and SEQ ID NO: 78 were digested with BamHI and XhoI, respectively, and ligated to the restriction sites BamHI and XhoI of plasmid 06.01.1812 (SEQ ID NO: 90), thereby constructing plasmids 06.01.1757 and 06.01.1807, respectively.

2. Construction of transposase mutant plasmid

Construction of TcBuster transposase mutant plasmid: The sequence encoding TcBuster #2 was constructed by ligating two DNA fragments (obtained by PCR amplification using plasmid 06.01.1614 as a template and using S3F and TcBmKE573-R, and TcBmKE573-F and S_IRES-R as primers, respectively) through fusion PCR. The sequence encoding TcBuster #3 was constructed by ligating two DNA fragments (obtained by PCR amplification using plasmid 06.01.1614 as a template and using S3F and TcBmA358-R, and TcBmA358-F and S_IRES-R as primers, respectively) through fusion PCR. The sequence encoding TcBuster #4 was constructed by ligating two DNA fragments (obtained by PCR amplification using plasmid 06.01.1614 as a template and using S3F and TcBmI452-R, and TcBmI452-F and S_IRES-R as primers, respectively) through fusion PCR. The sequence encoding TcBuster #5 was constructed by ligating two DNA fragments (obtained by PCR amplification using plasmid 06.01.1614 as a template and using S3F and TcBmN85-R, and TcBmN85-F and S_IRES-R as primers, respectively) through fusion PCR. The above fragments (sequences encoding TcBuster #2, TcBuster #3, TcBuster #4 and TcBuster #5) were digested with enzymes ClaI and XhoI, respectively, and ligated to the restriction sites ClaI and XhoI of plasmid 06.01.1812 (SEQ ID NO: 90), thereby constructing plasmids 06.01.1681, 06.01.1696, 06.01.1703 and 06.01.1705, respectively.

Construction of Bombyx mori Yabusame-1 transposase mutant plasmid: The sequence encoding Yabusame-1 #3 was constructed by ligating three DNA fragments (obtained by PCR amplification using plasmid 06.01.1687 as a template and using S3F and C_YabF321D-R, C_YabF321D-F and C_YabSK-R and C_YabSK-F and S_IRES-R as primers, respectively) through fusion PCR. The sequence encoding Yabusame-1 #4 was subjected to PCR amplification using plasmid 06.01.1687 as a template and using C_optiHBm-F and S_IRES-R as primers. The sequence encoding Yabusame-1 #5 was subjected to PCR amplification using plasmid 06.01.1740 as a template and using C_optiHBm #2-F and S_IRES-R as primers. The above fragments (sequences encoding Yabusame-1 #3, Yabusame-1 #4 and Yabusame-1 #5) were digested with enzymes ClaI and XhoI, respectively, and ligated to the restriction sites ClaI and XhoI of plasmid 06.01.1812 (SEQ ID NO: 90), thereby constructing plasmids 06.01.1517, 06.01.1778 and 06.01.1795, respectively.

Construction of Xenopus tropicalis Uribo2 transposase mutant plasmid: The sequence encoding Uribo2 #3 was constructed by ligating four DNA fragments (obtained by PCR amplification using plasmid 06.01.1770 as a template and using S3F and C_XtUP148T-R, C_XtUP148T-F and C_XtUD359N-R, C_XtUD359N-F and C_XtUA462H-R, and C_XtUA462H-F and C_XtUF576R-R as primers, respectively) through fusion PCR. The sequence encoding Uribo2 #4 was constructed by ligating four DNA fragments (obtained by PCR amplification using plasmid 06.01.1770 as a template and using S3F and C_XtUS193K-R, C_XtUS193K-F and C_XtUD359N-R, C_XtUD359N-F and C_XtUA462H-R, and C_XtUA462H-F and S_IRES-R as primers, respectively) through fusion PCR. The sequence encoding Uribo2 #5 was subjected to PCR amplification using plasmid 06.01.1770 as a template and using C_XtU #1-F and S_IRES-R as primers. The sequence encoding Uribo2 #6 was subjected to PCR amplification using plasmid 06.01.1790 as a template and using C_XtU #2-F and S_IRES-R as primers. The above fragments (sequences encoding Uribo2 #3, Uribo2 #4, Uribo2 #5 and Uribo2 #6) were digested with enzymes ClaI and XhoI, respectively, and ligated to the restriction sites ClaI and XhoI of plasmid 06.01.1812 (SEQ ID NO: 90), thereby constructing plasmids 06.01.1850, 06.01.1862, 06.01.1872 and 06.01.1884, respectively.

Construction of SB transposase mutant plasmid: The sequence encoding SB100 #2 was constructed by ligating two DNA fragments (obtained by PCR amplification using plasmid 06.01.1807 as a template and using S3F and C_SB100 #2-R, and C_SB100 #2-F and S_IRES-R as primers, respectively) through fusion PCR. The above fragment was digested with enzymes ClaI and XhoI, and ligated to the restriction sites ClaI and XhoI of plasmid 06.01.1812 (SEQ ID NO: 90), thereby constructing plasmid 06.01.1941.

3. Construction of transposon plasmid: the synthesized sequences SEQ ID NO: 5, SEQ ID NO: 10, SEQ ID NO: 14, SEQ ID NO: 18, SEQ ID NO: 22, SEQ ID NO: 31, SEQ ID NO: 35, SEQ ID NO: 40, SEQ ID NO: 45, SEQ ID NO: 48, SEQ ID NO: 53, SEQ ID NO: 59, SEQ ID NO: 65, SEQ ID NO: 68, SEQ ID NO: 77, SEQ ID NO: 76, SEQ ID NO: 88 and SEQ ID NO: 89 were digested with enzymes NotI and AsiSI, respectively, and ligated to the restriction sites NotI and AsiSI of plasmid 06.01.1955 (SEQ ID NO: 91), thereby constructing plasmids 06.01.1939, 06.01.1946, 06.01.1952, 06.01.1957, 06.01.1958, 06.01.1982, 06.01.1985, 06.01.2014, 06.01.2016, 06.01.2018, 06.01.2029, 06.01.2037, 06.01.2052, 06.01.2056, 06.01.1917, 06.01.2072, 06.01.1367 and 06.01.2099, respectively. The synthesized sequence SEQ ID NO: 27 was digested with BbsI and AsiSI, and ligated to the restriction sites NotI and AsiSI of plasmid 06.01.1955 (SEQ ID NO: 91), thereby constructing plasmid 06.01.1967.

4. Construction of transposon plasmid having puromycin (Puro) resistance gene and hPGK-luciferase-ires-EGFP sequence: the synthesized sequences SEQ ID NO: 93 and SEQ ID NO: 92 were digested with SbfI/PacI and PacI/AscI, respectively, and ligated to the restriction sites SbfI and AscI of plasmids 06.01.1939, 06.01.1946, 06.01.1952, 06.01.1957, 06.01.1958, 06.01.1967, 06.01.1982, 06.01.1985, 06.01.2014, 06.01.2016, 06.01.2018, 06.01.2029, 06.01.2037, 06.01.2052, 06.01.2056, 06.01.1917, 06.01.2072, 06.01.1367 and 06.01.2099, thereby constructing plasmids 06.01.2141, 06.01.2143, 06.01.2148, 06.01.2170, 06.01.2206, 06.01.2218, 06.01.2229, 06.01.2250, 06.01.2259, 06.01.2263, 06.01.2273, 06.01.2277, 06.01.2286, 06.01.2289, 06.01.2305, 06.01.2331, 06.01.2336, 06.01.2343 and 06.01.2327, respectively.

5. Construction of transposon plasmid having hygromycin (Hygro) resistance gene and hPGK-luciferase-ires-EGFP sequence: the synthesized sequences SEQ ID NO: 94 and SEQ ID NO: 92 were digested with SbfI/PacI and PacI/AscI, respectively, and ligated to the restriction sites SbfI and AscI of plasmids 06.01.1939, 06.01.1946, 06.01.1952, 06.01.1957, 06.01.1958, 06.01.1967, 06.01.1982, 06.01.1985, 06.01.2014, 06.01.2016, 06.01.2018, 06.01.2029, 06.01.2037, 06.01.2052, 06.01.2056, 06.01.1917, 06.01.2072, 06.01.1367 and 06.01.2099, thereby constructing plasmids 06.01.3233, 06.01.2347, 06.01.3321, 06.01.3331, 06.01.3372, 06.01.2358, 06.01.3428, 06.01.3455, 06.01.3457, 06.01.2360, 06.01.2362, 06.01.2879, 06.01.2370, 06.01.2379, 06.01.2420, 06.01.2429, 06.01.3105, 06.01.2433 and 06.01.2430, respectively.

6. Construction of transposon plasmid having blasticidin resistance gene and hPGK-luciferase-ires-EGFP sequence: the synthesized sequences SEQ ID NO: 95 and SEQ ID NO: 92 were digested with SbfI/PacI and PacI/AscI, respectively, and ligated to the restriction sites SbfI and AscI of plasmid 06.01.1367, thereby constructing plasmid 06.01.3335.

Construction of transposon plasmid for stable lentivirus producing cell line: the synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1939, thereby constructing plasmids 06.01.4301, 06.01.4302, 06.01.4303 and 06.01.4304. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1946, thereby constructing plasmids 06.01.4305, 06.01.4306, 06.01.4307 and 06.01.4308. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1952, thereby constructing plasmids 06.01.4309, 06.01.4310, 06.01.4311 and 06.01.4312. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1957, thereby constructing plasmids 06.01.4313, 06.01.4314, 06.01.4315 and 06.01.4316. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1958, thereby constructing plasmids 06.01.4317, 06.01.4318, 06.01.4319 and 06.01.4320. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1967, thereby constructing plasmids 06.01.4321, 06.01.4322, 06.01.4323 and 06.01.4324. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1982, thereby constructing plasmids 06.01.4325, 06.01.4326, 06.01.4327 and 06.01.4328. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1985, thereby constructing plasmids 06.01.4329, 06.01.4330, 06.01.4331 and 06.01.4332. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.2014, thereby constructing plasmids 06.01.4333, 06.01.4334, 06.01.4335 and 06.01.4336. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.2016, thereby constructing plasmids 06.01.4337, 06.01.4338, 06.01.4339 and 06.01.4340. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.2029, thereby constructing plasmids 06.01.4341, 06.01.4342, 06.01.4343 and 06.01.4344. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.2037, thereby constructing plasmids 06.01.4345, 06.01.4346, 06.01.4347 and 06.01.4348. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.2052, thereby constructing plasmids 06.01.4349, 06.01.4350, 06.01.4351 and 06.01.4352. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1917, thereby constructing plasmids 06.01.4353, 06.01.4354, 06.01.4355 and 06.01.4356. The synthesized sequences SEQ ID NO: 96, SEQ ID NO: 97, SEQ ID NO: 98 and SEQ ID NO: 99 were digested with SbfI/AscI, respectively, and ligated to the restriction sites SbfI and AscI of plasmid 06.01.1367, thereby constructing plasmids 06.01.4357, 06.01.4358, 06.01.4359 and 06.01.4360. The synthesized sequence SEQ ID NO: 100 was digested with SbfI/AscI, and ligated to the restriction sites SbfI and AscI of plasmids 06.01.1939, 06.01.1946, 06.01.1952, 06.01.1957, 06.01.1958, 06.01.1967, 06.01.1982, 06.01.1985, 06.01.2014, 06.01.2016, 06.01.2029, 06.01.2037, 06.01.2052, 06.01.1917 and 06.01.1367, thereby constructing plasmids 06.01.4361, 06.01.4362, 06.01.4363, 06.01.4364, 06.01.4365, 06.01.4366, 06.01.4367, 06.01.4368, 06.01.4369, 06.01.4370, 06.01.4371, 06.01.4372, 06.01.4373, 06.01.4374 and 06.01.4375.

TABLE 1
Summary of sequences of the present disclosure

| Sequence number | Description |
|---|---|
| SEQ ID NO: 1 | Tol1 transposase plasmid clone sequence-GenScript synthesis |
| SEQ ID NO: 2 | Tol1 transposase amino acid sequence |
| SEQ ID NO: 3 | Tol1 transposon 5′ terminal inverted repeat |
| SEQ ID NO: 4 | Tol1 transposon 3′ terminal inverted repeat |
| SEQ ID NO: 5 | Tol1 transposon plasmid clone sequence-GenScript synthesis |
| SEQ ID NO: 6 | Tol2 transposase plasmid clone sequence-GenScript synthesis |
| SEQ ID NO: 7 | Tol2 transposase amino acid sequence |
| SEQ ID NO: 8 | Tol2 transposon 5′ terminal inverted repeat |
| SEQ ID NO: 9 | Tol2 transposon 3′ terminal inverted repeat |
| SEQ ID NO: 10 | Tol2 transposon plasmid clone sequence-GenScript synthesis |
| SEQ ID NO: 11 | Frog prince transposase plasmid clone sequence-GenScript synthesis |
| SEQ ID NO: 12 | Frog prince transposase amino acid sequence |
| SEQ ID NO: 13 | Frog prince transposon terminal inverted repeat |
| SEQ ID NO: 14 | Frog prince transposon plasmid clone sequence-GenScript synthesis |
| SEQ ID NO: 15 | Minos transposase plasmid clone sequence-GenScript synthesis |
| SEQ ID NO: 16 | Minos transposase amino acid sequence |
| SEQ ID NO: 17 | Minos transposon terminal inverted repeat |
| SEQ ID NO: 18 | Minos transposon plasmid clone sequence-GenScript synthesis |
| SEQ ID NO: 19 | Hsmar1 transposase plasmid clone sequence-GenScript synthesis |
| SEQ ID NO: 20 | Hsmar1 transposase amino acid sequence |
| SEQ ID NO: 21 | Hsmar1 transposon terminal inverted repeat |
| SEQ ID NO: 22 | Hsmar1 transposon plasmid clone sequence-GenScript synthesis |
| SEQ ID NO: 23 | Helraiser transposase plasmid clone sequence-GenScript synthesis |
| SEQ ID NO: 24 | Helraiser transposase amino acid sequence |
| SEQ ID NO: 25 | Helitron LTS left terminal sequence |
| SEQ ID NO: 26 | Helitron RTS right terminal sequence |
| SEQ ID NO: 27 | Helitron transposon plasmid clone sequence-GenScript synthesis |
| SEQ ID NO: 28 | ZB transposase plasmid clone sequence-GenScript synthesis |
| SEQ ID NO: 29 | ZB transposase amino acid sequence |
| SEQ ID NO: 30 | ZB transposon terminal inverted repeat |
| SEQ ID NO: 31 | ZB transposon plasmid clone sequence-GenScript synthesis |
| SEQ ID NO: 32 | Intruder (IT) transposase plasmid clone sequence-GenScript synthesis |
| SEQ ID NO: 33 | Intruder (IT) transposase amino acid sequence |
| SEQ ID NO: 34 | Intruder (IT) transposon terminal inverted repeat |
| SEQ ID NO: 35 | Intruder (IT) transposon plasmid clone sequence-GenScript synthesis |
| SEQ ID NO: 36 | SPIN_{ON} transposase plasmid clone sequence-GenScript synthesis |
| SEQ ID NO: 37 | SPIN_{ON} transposase amino acid sequence |
| SEQ ID NO: 38 | SPIN_{ON} transposon 5′ terminal inverted repeat |
| SEQ ID NO: 39 | SPIN_{ON} transposon 3′ terminal inverted repeat |
| SEQ ID NO: 40 | SPIN_{ON} transposon plasmid clone sequence-GenScript synthesis |
| SEQ ID NO: 41 | TcBusterM3 (D189A/V377T/E469K) transposase plasmid clone sequence-GenScript synthesis |
| SEQ ID NO: 42 | TcBuster transposase TcBuster_{co} amino acid sequence |
| SEQ ID NO: 43 | TcBuster transposon 5′ terminal inverted repeat #1 |
| SEQ ID NO: 44 | TcBuster transposon 3′ terminal inverted repeat #1 |
| SEQ ID NO: 45 | TcBuster transposon plasmid clone sequence #1-GenScript synthesis |
| SEQ ID NO: 46 | TcBuster transposon 5′ terminal inverted repeat #2 |
| SEQ ID NO: 47 | TcBuster transposon 3′ terminal inverted repeat #2 |
| SEQ ID NO: 48 | TcBuster transposon plasmid clone sequence #2-GenScript synthesis |
| SEQ ID NO: 49 | Passport (PT) transposase plasmid clone sequence-GenScript synthesis |
| SEQ ID NO: 50 | Passport (PT) transposase amino acid sequence |
| SEQ ID NO: 51 | Passport (PT) transposon 5′ terminal inverted repeat |
| SEQ ID NO: 52 | Passport (PT) transposon 3′ terminal inverted repeat |
| SEQ ID NO: 53 | Passport (PT) transposon plasmid clone sequence-GenScript synthesis |
| SEQ ID NO: 54 | Bombyx mori yabusame-1 transposase #1 plasmid clone sequence-GenScript synthesis |
| SEQ ID NO: 55 | Bombyx mori yabusame-1 transposase #2 plasmid clone sequence-GenScript synthesis |
| SEQ ID NO: 56 | Bombyx mori yabusame-1 transposase #1 amino acid sequence (GenBank accession number BAD11135.1) |
| SEQ ID NO: 57 | Bombyx mori yabusame-1 transposon 5′ terminal inverted repeat |
| SEQ ID NO: 58 | Bombyx mori yabusame-1 transposon 3′ terminal inverted repeat |
| SEQ ID NO: 59 | Bombyx mori yabusame-1 transposon plasmid clone sequence-GenScript synthesis |
| SEQ ID NO: 60 | Xenopus tropicalis Uribo2 transposase #1 plasmid clone sequence-GenScript synthesis |
| SEQ ID NO: 61 | Xenopus tropicalis Uribo2 transposase #2 plasmid clone sequence-GenScript synthesis |
| SEQ ID NO: 62 | Xenopus tropicalis Uribo2 transposase #1 amino acid sequence (GenBank accession number BAF82022) |
| SEQ ID NO: 63 | Xenopus tropicalis Uribo2 transposon 5′ terminal inverted repeat #1 |
| SEQ ID NO: 64 | Xenopus tropicalis Uribo2 transposon 3′ terminal inverted repeat #1 |
| SEQ ID NO: 65 | Xenopus tropicalis Uribo2 transposon plasmid clone sequence #1-GenScript synthesis |
| SEQ ID NO: 66 | Xenopus tropicalis Uribo2 transposon 5′ terminal inverted repeat #2 |
| SEQ ID NO: 67 | Xenopus tropicalis Uribo2 transposon 3′ terminal inverted repeat #2 |
| SEQ ID NO: 68 | Xenopus tropicalis Uribo2 transposon plasmid clone sequence #2-GenScript synthesis |
| SEQ ID NO: 69 | piggyBac transposase #1 plasmid clone sequence-GenScript synthesis |
| SEQ ID NO: 70 | HypiggyBac transposase #2 plasmid clone sequence-GenScript synthesis |
| SEQ ID NO: 71 | HypiggyBac transposase #3 plasmid clone sequence-GenScript synthesis |
| SEQ ID NO: 72 | HypiggyBac transposase #4 plasmid clone sequence-GenScript synthesis |
| SEQ ID NO: 73 | piggyBac#1 transposase amino acid sequence (GenBank accession number ABC67521) |
| SEQ ID NO: 74 | piggyBac transposon minimal 5′ terminal inverted repeat |
| SEQ ID NO: 75 | piggyBac transposon minimal 3′ terminal inverted repeat |
| SEQ ID NO: 76 | piggyBac minimal TIR transposon plasmid clone sequence-GenScript synthesis |
| SEQ ID NO: 77 | piggyBac transposon plasmid clone sequence-GenScript synthesis |
| SEQ ID NO: 78 | SB100 transposase #1 plasmid clone sequence (BamHI + XhoI)-GenScript synthesis |
| SEQ ID NO: 79 | SB100 transposase amino acid sequence |
| SEQ ID NO: 80 | SB10 transposase amino acid sequence |
| SEQ ID NO: 81 | SB transposon PT 5′ terminal inverted repeat |
| SEQ ID NO: 82 | SB transposon PT 3′ terminal inverted repeat |
| SEQ ID NO: 83 | SB transposon PT2 5′ terminal inverted repeat |
| SEQ ID NO: 84 | SB transposon PT2 3′ terminal inverted repeat |
| SEQ ID NO: 85 | SB transposon PT3 terminal inverted repeat |
| SEQ ID NO: 86 | SB transposon PT4 5′ terminal inverted repeat |
| SEQ ID NO: 87 | SB transposon PT4 3′ terminal inverted repeat |
| SEQ ID NO: 88 | SB transposon PT3 plasmid clone sequence-GenScript synthesis |
| SEQ ID NO: 89 | SB transposon PT4 plasmid clone sequence-GenScript synthesis |
| SEQ ID NO: 90 | 06.01.1812_(A)pmaCMV(BGI)-MCS-IRES-ECFP plasmid sequence-GenScript synthesis |
| SEQ ID NO: 91 | 06.01.1955_(A)pmk-MCS plasmid sequence-GenScript synthesis |
| SEQ ID NO: 92 | PacI-hPGK-Luciferase-ires-EGFP-WPRE-AscI insert-GenScript synthesis |
| SEQ ID NO: 93 | SbfI-PuroR(R)-PacI insert-GenScript synthesis |
| SEQ ID NO: 94 | SbfI-HygroR(R)-PacI insert-GenScript synthesis |
| SEQ ID NO: 95 | SbfI-BSD(R)-PacI insert-GenScript synthesis |
| SEQ ID NO: 96 | SbfI-CMV(BGI)-gag/pol-AscI insert-GenScript synthesis |
| SEQ ID NO: 97 | SbfI-TRE2CuO-rev-AscI insert-GenScript synthesis |
| SEQ ID NO: 98 | SbfI-TRE2CuO(BGI)-VSVG-AscI insert-GenScript synthesis |
| SEQ ID NO: 99 | SbfI-CAGGS(BGIC&R)-rtTA-CMV(BGI)-CymR-HygroR-AscI insert-GenScript synthesis |
| SEQ ID NO: 100 | SbfI-LVRSV-hPGK-Luciferase-ires-EGFP-PuroR-AscI insert-GenScript synthesis |

TABLE 2
Primers for plasmid construction

| Sequence number | Primer name | Sequence |
|---|---|---|
| SEQ ID NO: 101 | S3F | ATTCTGAGTCCAAGCTAGGC |
| SEQ ID NO: 102 | S_IRES-R | GGGCCCTCACATTGCCA |
| SEQ ID NO: 103 | TcBmN85-F | GCAACAGCTCCCTGAATCCTGC |
| SEQ ID NO: 104 | TcBmN85-R | GAGCTGTTGCTCACCACCTGTTCGCA |
| SEQ ID NO: 105 | TcBmA358-F | AGAGTGTTCAAGCTGCTGTGCGACGATCTG |
| SEQ ID NO: 106 | TcBmA358-R | CACAGCAGCTTGAACACTCTGGCGTTCAGA |
| SEQ ID NO: 107 | TcBml452-F | TCAATAGCAAGAAAAGCAAGCTGAAGCTCTG |
| SEQ ID NO: 108 | TcBml452-R | CAGCTTGCTTTTCTTGCTATTGATTCTGCTGTTC |
| SEQ ID NO: 109 | TcBmKE573-F | CTGAGAGAGCCGTGAAGCTGCTGATGCCTTTTGTGACCA |
| SEQ ID NO: 110 | TcBmKE573-R | CAGCAGCTTCACGGCTCTCTCAGAAATCTCGGGGAATTC |
| SEQ ID NO: 111 | C_YabF321D-F | CGCCAAGAATGACTACGTGGTCAACCTGGA |
| SEQ ID NO: 112 | C_YabF321D-R | TGACCACGTAGTCATTCTTGGCGTCCACCAG |
| SEQ ID NO: 113 | C_YabSK-F | AGCAGACACGTGAACGTGAAGGGAAGATACGTGAGGTGTC |
| SEQ ID NO: 114 | C_YabSK-R | CTTCCCTTCACGTTCACGTGTCTGCTGGAAGGCTCGCCCAGCT |
| SEQ ID NO: 115 | C_optiHBm-F | gcgaatcgatgccaccATGGACATCGAGAGACAGGAAG |
| SEQ ID NO: 116 | C_optiHBm#2-F | gcgaatcgatgccaccATGGATATCGAGCGGCAGGA |
| SEQ ID NO: 117 | C_XtUP148T-F | CAGAATCCTCTGACCAGATACGCCAGAGCACAT |
| SEQ ID NO: 118 | C_XtUP148T-R | TCTGGCGTATCTGGTCAGAGGATTCTGGGTCAG |
| SEQ ID NO: 119 | C_XtUD359N-F | CTGTACTGCCTGAACACACCTGCTTGTGGCAC |
| SEQ ID NO: 120 | C_XtUD359N-R | AGCAGGTGTGTTCAGGCAGTACAGGGCGGTA |
| SEQ ID NO: 121 | C_XtUA462H-F | AGGAAGACCAGACACTGGTACAAAAAAGTGGGAATCT |
| SEQ ID NO: 122 | C_XtUA462H-R | CACTTTTTTGTACCAGTGTCTGGTCTTCCTGGTGG |
| SEQ ID NO: 123 | C_XtUF576R-R | gactctcgagTTATCAGTAGTGCAGCTGGGTGTGGTAGATCTCGAAA |
| | | CAGGGCTTCCGGCACAGGCCGGGGTT |
| SEQ ID NO: 124 | C_XtUS193K-F | CAGTCCTGAAGATCCCCGTGTTCTCTGCT |
| SEQ ID NO: 125 | C_XtUS193K-R | AACACGGGGATCTTCAGGACTGTTGTGGTGT |
| SEQ ID NO: 126 | C_XtU#1-F | gcgaatcgatgccaccATGGCCAAGAGATTCTACAGC |
| SEQ ID NO: 127 | C_XtU#2-F | gcgaatcgatgccaccATGGCCAAGCGGTTCTACAG |
| SEQ ID NO: 128 | C_SB100#2-F | AGATCGACGGCAGCATGGATGCCGTGCAGTA |
| SEQ ID NO: 129 | C_SB100#2-R | CATCCATGCTGCCGTCGATCTTGTGCA |
| SEQ ID NO: 130 | WPRE-taqman-F | CCTTTCCGGGACTTTCGCTTT |
| SEQ ID NO: 131 | WPRE-taqman-R | GCAGAATCCAGGTGGCAACA |
| SEQ ID NO: 132 | WPRE-Probe | FAM-ACTCATCGCCGCCTGCCTTGCC-MGB |

TABLE 3
Plasmid name and construction information

| Number | Plasmid name | Insert | Cloning site | Vector |
|---|---|---|---|---|
| 06.01.1494 | (A)pmaCMV(BGI)-Tollase-IRES-ECFP | SEQ ID NO: 1 | ClaI + XhoI | 06.01.1812 |
| 06.01.1504 | (A)pmaCMV(BGI)-Tol2 ase-IRES-ECFP | SEQ ID NO: 6 | ClaI + XhoI | 06.01.1812 |
| 06.01.1527 | (A)pmaCMV(BGI)-FPase-IRES-ECFP | SEQ ID NO: 11 | ClaI + XhoI | 06.01.1812 |
| 06.01.1535 | (A)pmaCMV(BGI)-Minos1-IRES-ECFP | SEQ ID NO: 15 | ClaI + XhoI | 06.01.1812 |
| 06.01.1538 | (A)pmaCMV(BGI)-Hsmar1-IRES-ECFP | SEQ ID NO: 19 | ClaI + XhoI | 06.01.1812 |
| 06.01.1579 | (A)pmaCMV(BGI)-Helraiser-IRES-ECFP | SEQ ID NO: 23 | ClaI + XhoI | 06.01.1812 |
| 06.01.1573 | (A)pmaCMV(BGI)-ZBase-IRES-ECFP | SEQ ID NO: 28 | ClaI + XhoI | 06.01.1812 |
| 06.01.1582 | (A)pmaCMV(BGI)-Intruder(ITase)-IRES-ECFP | SEQ ID NO: 32 | ClaI + XhoI | 06.01.1812 |
| 06.01.1596 | (A)pmaCMV(BGI)-SPINon-IRES-ECFP | SEQ ID NO: 36 | ClaI + XhoI | 06.01.1812 |
| 06.01.1614 | (A)pmaCMV(BGI)-TcBuster#1-IRES-ECFP | SEQ ID NO: 41 | ClaI + XhoI | 06.01.1812 |
| 06.01.1681 | (A)pmaCMV(BGI)-TcBuster#2-IRES-ECFP | Fusion PCR | ClaI + XhoI | 06.01.1812 |
| 06.01.1696 | (A)pmaCMV(BGI)-TcBuster#3-IRES-ECFP | Fusion PCR | ClaI + XhoI | 06.01.1812 |
| 06.01.1703 | (A)pmaCMV(BGI)-TcBuster#4-IRES-ECFP | Fusion PCR | ClaI + XhoI | 06.01.1812 |
| 06.01.1705 | (A)pmaCMV(BGI)-TcBuster#5-IRES-ECFP | Fusion PCR | ClaI + XhoI | 06.01.1812 |
| 06.01.1679 | (A)pmaCMV(BGI)-PTase-IRES-ECFP | SEQ ID NO: 49 | ClaI + XhoI | 06.01.1812 |
| 06.01.1687 | (A)pmaCMV(BGI)-Yabusame-1#1-IRES-ECFP | SEQ ID NO: 54 | ClaI + XhoI | 06.01.1812 |
| 06.01.1740 | (A)pmaCMV(BGI)-Yabusame-1#2-IRES-ECFP | SEQ ID NO: 55 | ClaI + XhoI | 06.01.1812 |
| 06.01.1517 | (A)pmaCMV(BGI)-Yabusame-1#3-IRES-ECFP | Fusion PCR | ClaI + XhoI | 06.01.1812 |
| 06.01.1778 | (A)pmaCMV(BGI)-Yabusame-1#4-IRES-ECFP | PCR | ClaI + XhoI | 06.01.1812 |
| 06.01.1795 | (A)pmaCMV(BGI)-Yabusame-1#5-IRES-ECFP | PCR | ClaI + XhoI | 06.01.1812 |
| 06.01.1770 | (A)pmaCMV(BGI)-Uribo2#1-IRES-ECFP | SEQ ID NO: 60 | ClaI + XhoI | 06.01.1812 |
| 06.01.1790 | (A)pmaCMV(BGI)-Uribo2#2-IRES-ECFP | SEQ ID NO: 61 | ClaI + XhoI | 06.01.1812 |
| 06.01.1850 | (A)pmaCMV(BGI)-Uribo2#3-IRES-ECFP | Fusion PCR | ClaI + XhoI | 06.01.1812 |
| 06.01.1862 | (A)pmaCMV(BGI)-Uribo2#4-IRES-ECFP | Fusion PCR | ClaI + XhoI | 06.01.1812 |
| 06.01.1872 | (A)pmaCMV(BGI)-Uribo2#5-IRES-ECFP | PCR | ClaI + XhoI | 06.01.1812 |
| 06.01.1884 | (A)pmaCMV(BGI)-Uribo2#6-IRES-ECFP | PCR | ClaI + XhoI | 06.01.1812 |
| 06.01.1757 | (A)pmaCMV(BGI)-HyPiggyBac#2-IRES-ECFP | SEQ ID NO: 70 | BamHI + XhoI | 06.01.1812 |
| 06.01.1581 | pmaCMV(BGI)-PiggyBac#1-IRES-ECFP | SEQ ID NO: 69 | ClaI + XhoI | 06.01.1812 |
| 06.01.1892 | (A)pmaCMV(BGI)-HyPiggyBac#3-IRES-ECFP | SEQ ID NO: 72 | ClaI + XhoI | 06.01.1812 |
| 06.01.1903 | (A)pmaCMV(BGI)-HyPiggyBac#4-IRES-ECFP | SEQ ID NO: 71 | ClaI + XhoI | 06.01.1812 |
| 06.01.1807 | (A)pmaCMV(BGI)-SB100#1-IRES-ECFP | SEQ ID NO: 78 | BamHI + XhoI | 06.01.1812 |
| 06.01.1941 | (A)pmaCMV(BGI)-SB100#2-IRES-ECFP | Fusion PCR | BamHI + XhoI | 06.01.1812 |
| 06.01.1812 | (A)pmaCMV(BGI)-MCS-IRES-ECFP | N/A | N/A | SEQ ID NO: 90 |
| 06.01.1939 | (A)pmkTo1T-MCS | SEQ ID NO: 5 | NotI + AsiSI | 06.01.1955 |
| 06.01.1946 | (A)pmkTo2T-MCS | SEQ ID NO: 10 | NotI + AsiSI | 06.01.1955 |
| 06.01.1952 | (A)pmkFTP-MCS | SEQ ID NO: 14 | NotI + AsiSI | 06.01.1955 |
| 06.01.1957 | (A)pmkMiT-MCS | SEQ ID NO: 18 | NotI + AsiSI | 06.01.1955 |
| 06.01.1958 | (A)pmkHsmT-MCS | SEQ ID NO: 22 | NotI + AsiSI | 06.01.1955 |
| 06.01.1967 | (A)pmkHelT-MCS | SEQ ID NO: 27 | NotI + AsiSI | 06.01.1955 |
| 06.01.1982 | (A)pmkZBT-MCS | SEQ ID NO: 31 | NotI + AsiSI | 06.01.1955 |
| 06.01.1985 | (A)pmkITT-MCS | SEQ ID NO: 35 | NotI + AsiSI | 06.01.1955 |
| 06.01.2014 | (A)pmkSPINT-MCS | SEQ ID NO: 40 | NotI + AsiSI | 06.01.1955 |
| 06.01.2016 | (A)pmkTcBT1-MCS | SEQ ID NO: 45 | NotI + AsiSI | 06.01.1955 |
| 06.01.2018 | (A)pmkTcBT2-MCS | SEQ ID NO: 48 | NotI + AsiSI | 06.01.1955 |
| 06.01.2029 | (A)pmkPTT-MCS | SEQ ID NO: 53 | NotI + AsiSI | 06.01.1955 |
| 06.01.2037 | (A)pmkBmYT-MCS | SEQ ID NO: 59 | NotI + AsiSI | 06.01.1955 |
| 06.01.2052 | (A)pmkXtUT1-MCS | SEQ ID NO: 65 | NotI + AsiSI | 06.01.1955 |
| 06.01.2056 | (A)pmkXtUT2-MCS | SEQ ID NO: 68 | NotI + AsiSI | 06.01.1955 |
| 06.01.1917 | (A)pmkHPBT-MCS | SEQ ID NO: 77 | NotI + AsiSI | 06.01.1955 |
| 06.01.2072 | (A)pmkmPBT-MCS | SEQ ID NO: 76 | NotI + AsiSI | 06.01.1955 |
| 06.01.1367 | (A)pmkSBT3-MCS | SEQ ID NO: 88 | NotI + AsiSI | 06.01.1955 |
| 06.01.2099 | (A)pmkSBT4-MCS | SEQ ID NO: 89 | NotI + AsiSI | 06.01.1955 |
| 06.01.1955 | (A)pmk-MCS | N/A | N/A | SEQ ID NO: 91 |
| 06.01.2141 | pmkTo1T-PuroR(R)-hPGK-Luciferase-ires-EGFP-WPRE | SEQ ID NO: 92 & 93 | SbfI + AscI | 06.01.1939 |
| 06.01.2143 | pmkTo2T-PuroR(R)-hPGK-Luciferase-ires-EGFP-WPRE | SEQ ID NO: 92 & 93 | SbfI + AscI | 06.01.1946 |
| 06.01.2148 | pmkFTP-PuroR(R)-hPGK-Luciferase-ires-EGFP-WPRE | SEQ ID NO: 92 & 93 | SbfI + AscI | 06.01.1952 |
| 06.01.2170 | pmkMiT-PuroR(R)-hPGK-Luciferase-ires-EGFP-WPRE | SEQ ID NO: 92 & 93 | SbfI + AscI | 06.01.1957 |
| 06.01.2206 | pmkHsmT-PuroR(R)-hPGK-Luciferase-ires-EGFP-WPRE | SEQ ID NO: 92 & 93 | SbfI + AscI | 06.01.1958 |
| 06.01.2218 | pmkHelT-PuroR(R)-hPGK-Luciferase-ires-EGFP-WPRE | SEQ ID NO: 92 & 93 | SbfI + AscI | 06.01.1967 |
| 06.01.2229 | pmkZBT-PuroR(R)-hPGK-Luciferase-ires-EGFP-WPRE | SEQ ID NO: 92 & 93 | SbfI + AscI | 06.01.1982 |
| 06.01.2250 | pmkITT-PuroR(R)-hPGK-Luciferase-ires-EGFP-WPRE | SEQ ID NO: 92 & 93 | SbfI + AscI | 06.01.1985 |
| 06.01.2259 | pmkSPINT-PuroR(R)-hPGK-Luciferase-ires-EGFP-WPRE | SEQ ID NO: 92 & 93 | SbfI + AscI | 06.01.2014 |
| 06.01.2263 | pmkTcBT1-PuroR(R)-hPGK-Luciferase-ires-EGFP-WPRE | SEQ ID NO: 92 & 93 | SbfI + AscI | 06.01.2016 |
| 06.01.2273 | pmkTcBT2-PuroR(R)-hPGK-Luciferase-ires-EGFP-WPRE | SEQ ID NO: 92 & 93 | SbfI + AscI | 06.01.2018 |
| 06.01.2277 | pmkPTT-PuroR(R)-hPGK-Luciferase-ires-EGFP-WPRE | SEQ ID NO: 92 & 93 | SbfI + AscI | 06.01.2029 |
| 06.01.2286 | pmkBmYT-PuroR(R)-hPGK-Luciferase-ires-EGFP-WPRE | SEQ ID NO: 92 & 93 | SbfI + AscI | 06.01.2037 |
| 06.01.2289 | pmkXtUT1-PuroR(R)-hPGK-Luciferase-ires-EGFP-WPRE | SEQ ID NO: 92 & 93 | SbfI + AscI | 06.01.2052 |
| 06.01.2305 | pmkXtUT2-PuroR(R)-hPGK-Luciferase-ires-EGFP-WPRE | SEQ ID NO: 92 & 93 | SbfI + AscI | 06.01.2056 |
| 06.01.2331 | pmkHPBT-PuroR(R)-hPGK-Luciferase-ires-EGFP-WPRE | SEQ ID NO: 92 & 93 | SbfI + AscI | 06.01.1917 |
| 06.01.2336 | pmkmPBT-PuroR(R)-hPGK-Luciferase-ires-EGFP-WPRE | SEQ ID NO: 92 & 93 | SbfI + AscI | 06.01.2072 |
| 06.01.2343 | pmkSBT3-PuroR(R)-hPGK-Luciferase-ires-EGFP-WPRE | SEQ ID NO: 92 & 93 | SbfI + AscI | 06.01.1367 |
| 06.01.2327 | pmkSBT4-PuroR(R)-hPGK-Luciferase-ires-EGFP-WPRE | SEQ ID NO: 92 & 93 | SbfI + AscI | 06.01.2099 |
| 06.01.3233 | pmkTo1T-HygroR(R)-hPGK-Luciferase-ires-EGFP-WPRE | SEQ ID NO: 92 & 94 | SbfI + AscI | 06.01.1939 |
| 06.01.2347 | pmkTo2T-HygroR(R)-hPGK-Luciferase-ires-EGFP-WPRE | SEQ ID NO: 92 & 94 | SbfI + AscI | 06.01.1946 |
| 06.01.3321 | pmkFTP-HygroR(R)-hPGK-Luciferase-ires-EGFP-WPRE | SEQ ID NO: 92 & 94 | SbfI + AscI | 06.01.1952 |
| 06.01.3331 | pmkMiT-HygroR(R)-hPGK-Luciferase-ires-EGFP-WPRE | SEQ ID NO: 92 & 94 | SbfI + AscI | 06.01.1957 |
| 06.01.3372 | pmkHsmT-HygroR(R)-hPGK-Luciferase-ires-EGFP-WPRE | SEQ ID NO: 92 & 94 | SbfI + AscI | 06.01.1958 |
| 06.01.2358 | pmkHelT-HygroR(R)-hPGK-Luciferase-ires-EGFP-WPRE | SEQ ID NO: 92 & 94 | SbfI + AscI | 06.01.1967 |
| 06.01.3428 | pmkZBT-HygroR(R)-hPGK-Luciferase-ires-EGFP-WPRE | SEQ ID NO: 92 & 94 | SbfI + AscI | 06.01.1982 |
| 06.01.3455 | pmkITT-HygroR(R)-hPGK-Luciferase-ires-EGFP-WPRE | SEQ ID NO: 92 & 94 | SbfI + AscI | 06.01.1985 |
| 06.01.3457 | pmkSPINT-HygroR(R)-hPGK-Luciferase-ires-EGFP-WPRE | SEQ ID NO: 92 & 94 | SbfI + AscI | 06.01.2014 |
| 06.01.2360 | pmkTcBT1-HygroR(R)-hPGK-Luciferase-ires-EGFP-WPRE | SEQ ID NO: 92 & 94 | SbfI + AscI | 06.01.2016 |
| 06.01.2362 | pmkTcBT2-HygroR(R)-hPGK-Luciferase-ires-EGFP-WPRE | SEQ ID NO: 92 & 94 | SbfI + AscI | 06.01.2018 |
| 06.01.2879 | pmkPTT-HygroR(R)-hPGK-Luciferase-ires-EGFP-WPRE | SEQ ID NO: 92 & 94 | SbfI + AscI | 06.01.2029 |
| 06.01.2370 | pmkBmYT-HygroR(R)-hPGK-Luciferase-ires-EGFP-WPRE | SEQ ID NO: 92 & 94 | SbfI + AscI | 06.01.2037 |
| 06.01.2379 | pmkXtUT1-HygroR(R)-hPGK-Luciferase-ires-EGFP-WPRE | SEQ ID NO: 92 & 94 | SbfI + AscI | 06.01.2052 |
| 06.01.2420 | pmkXtUT2-HygroR(R)-hPGK-Luciferase-ires-EGFP-WPRE | SEQ ID NO: 92 & 94 | SbfI + AscI | 06.01.2056 |
| 06.01.2429 | pmkHPBT-HygroR(R)-hPGK-Luciferase-ires-EGFP-WPRE | SEQ ID NO: 92 & 94 | SbfI + AscI | 06.01.1917 |
| 06.01.3105 | pmkmPBT-HygroR(R)-hPGK-Luciferase-ires-EGFP-WPRE | SEQ ID NO: 92 & 94 | SbfI + AscI | 06.01.2072 |
| 06.01.2433 | pmkSBT3-HygroR(R)-hPGK-Luciferase-ires-EGFP-WPRE | SEQ ID NO: 92 & 94 | SbfI + AscI | 06.01.1367 |
| 06.01.2430 | pmkSBT4-HygroR(R)-hPGK-Luciferase-ires-EGFP-WPRE | SEQ ID NO: 92 & 94 | SbfI + AscI | 06.01.2099 |
| 06.01.3335 | pmkSBT3-BSD(R)-hPGK-Luciferase-ires-EGFP-WPRE | SEQ ID NO: 92 & 95 | SbfI + AscI | 06.01.1367 |
| 06.01.4301 | (A)pmkTo1T-CMV(BGI)-gag/pol | SEQ ID NO: 96 | SbfI + AscI | 06.01.1939 |
| 06.01.4302 | (A)pmkTo1T-TRE2CuO-rev | SEQ ID NO: 97 | SbfI + AscI | 06.01.1939 |
| 06.01.4303 | (A)pmkTo1T-TRE2CuO(BGI)-VSVG | SEQ ID NO: 98 | SbfI + AscI | 06.01.1939 |
| 06.01.4304 | (A)pmkTo1T-CAGGS(BGIC&R)-rtTA-CMV(BGI)-CymR-HygroR | SEQ ID NO: 99 | SbfI + AscI | 06.01.1939 |
| 06.01.4305 | (A)pmkTo2T-CMV(BGI)-gag/pol | SEQ ID NO: 96 | SbfI + AscI | 06.01.1946 |
| 06.01.4306 | (A)pmkTo2T-TRE2CuO-rev | SEQ ID NO: 97 | SbfI + AscI | 06.01.1946 |
| 06.01.4307 | (A)pmkTo2T-TRE2CuO(BGI)-VSVG | SEQ ID NO: 98 | SbfI + AscI | 06.01.1946 |
| 06.01.4308 | (A)pmkTo2T-CAGGS(BGIC&R)-rtTA-CMV(BGI)-CymR-HygroR | SEQ ID NO: 99 | SbfI + AscI | 06.01.1946 |
| 06.01.4309 | (A)pmkFTP-CMV(BGI)-gag/pol | SEQ ID NO: 96 | SbfI + AscI | 06.01.1952 |
| 06.01.4310 | (A)pmkFTP-TRE2CuO-rev | SEQ ID NO: 97 | SbfI + AscI | 06.01.1952 |
| 06.01.4311 | (A)pmkFTP-TRE2CuO(BGI)-VSVG | SEQ ID NO: 98 | SbfI + AscI | 06.01.1952 |
| 06.01.4312 | (A)pmkFTP-CAGGS(BGIC&R)-rtTA-CMV(BGI)-CymR-HygroR | SEQ ID NO: 99 | SbfI + AscI | 06.01.1952 |
| 06.01.4313 | (A)pmkMiT-CMV(BGI)-gag/pol | SEQ ID NO: 96 | SbfI + AscI | 06.01.1957 |
| 06.01.4314 | (A)pmkMiT-TRE2CuO-rev | SEQ ID NO: 97 | SbfI + AscI | 06.01.1957 |
| 06.01.4315 | (A)pmkMiT-TRE2CuO(BGI)-VSVG | SEQ ID NO: 98 | SbfI + AscI | 06.01.1957 |
| 06.01.4316 | (A)pmkMiT-CAGGS(BGIC&R)-rtTA-CMV(BGI)-CymR-HygroR | SEQ ID NO: 99 | SbfI + AscI | 06.01.1957 |
| 06.01.4317 | (A)pmkHsmT-CMV(BGI)-gag/pol | SEQ ID NO: 96 | SbfI + AscI | 06.01.1958 |
| 06.01.4318 | (A)pmkHsmT-TRE2CuO-rev | SEQ ID NO: 97 | SbfI + AscI | 06.01.1958 |
| 06.01.4319 | (A)pmkHsmT-TRE2CuO(BGI)-VSVG | SEQ ID NO: 98 | SbfI + AscI | 06.01.1958 |
| 06.01.4320 | (A)pmkHsmT-CAGGS(BGIC&R)-rtTA-CMV(BGI)-CymR-HygroR | SEQ ID NO: 99 | SbfI + AscI | 06.01.1958 |
| 06.01.4321 | (A)pmkHelT-CMV(BGI)-gag/pol | SEQ ID NO: 96 | SbfI + AscI | 06.01.1967 |
| 06.01.4322 | (A)pmkHelT-TRE2CuO-rev | SEQ ID NO: 97 | SbfI + AscI | 06.01.1967 |
| 06.01.4323 | (A)pmkHelT-TRE2CuO(BGI)-VSVG | SEQ ID NO: 98 | SbfI + AscI | 06.01.1967 |
| 06.01.4324 | (A)pmkHelT-CAGGS(BGIC&R)-rtTA-CMV(BGI)-CymR-HygroR | SEQ ID NO: 99 | SbfI + AscI | 06.01.1967 |
| 06.01.4325 | (A)pmkZBT-CMV(BGI)-gag/pol | SEQ ID NO: 96 | SbfI + AscI | 06.01.1982 |
| 06.01.4326 | (A)pmkZBT-TRE2CuO-rev | SEQ ID NO: 97 | SbfI + AscI | 06.01.1982 |
| 06.01.4327 | (A)pmkZBT-TRE2CuO(BGI)-VSVG | SEQ ID NO: 98 | SbfI + AscI | 06.01.1982 |
| 06.01.4328 | (A)pmkZBT-CAGGS(BGIC&R)-rtTA-CMV(BGI)-CymR-HygroR | SEQ ID NO: 99 | SbfI + AscI | 06.01.1982 |
| 06.01.4329 | (A)pmkITT-CMV(BGI)-gag/pol | SEQ ID NO: 96 | SbfI + AscI | 06.01.1985 |
| 06.01.4330 | (A)pmkITT-TRE2CuO-rev | SEQ ID NO: 97 | SbfI + AscI | 06.01.1985 |
| 06.01.4331 | (A)pmkITT-TRE2CuO(BGI)-VSVG | SEQ ID NO: 98 | SbfI + AscI | 06.01.1985 |
| 06.01.4332 | (A)pmkITT-CAGGS(BGIC&R)-rtTA-CMV(BGI)-CymR-HygroR | SEQ ID NO: 99 | SbfI + AscI | 06.01.1985 |
| 06.01.4333 | (A)pmkSPINT-CMV(BGI)-gag/pol | SEQ ID NO: 96 | SbfI + AscI | 06.01.2014 |
| 06.01.4334 | (A)pmkSPINT-TRE2CuO-rev | SEQ ID NO: 97 | SbfI + AscI | 06.01.2014 |
| 06.01.4335 | (A)pmkSPINT-TRE2CuO(BGI)-VSVG | SEQ ID NO: 98 | SbfI + AscI | 06.01.2014 |
| 06.01.4336 | (A)pmkSPINT-CAGGS(BGIC&R)-rtTA-CMV(BGI)-CymR-HygroR | SEQ ID NO: 99 | SbfI + AscI | 06.01.2014 |
| 06.01.4337 | (A)pmkTcBT1-CMV(BGI)-gag/pol | SEQ ID NO: 96 | SbfI + AscI | 06.01.2016 |
| 06.01.4338 | (A)pmkTcBT1-TRE2CuO-rev | SEQ ID NO: 97 | SbfI + AscI | 06.01.2016 |
| 06.01.4339 | (A)pmkTcBT1-TRE2CuO(BGI)-VSVG | SEQ ID NO: 98 | SbfI + AscI | 06.01.2016 |
| 06.01.4340 | (A)pmkTcBT1-CAGGS(BGIC&R)-rtTA-CMV(BGI)-CymR-HygroR | SEQ ID NO: 99 | SbfI + AscI | 06.01.2016 |
| 06.01.4341 | (A)pmkPTT-CMV(BGI)-gag/pol | SEQ ID NO: 96 | SbfI + AscI | 06.01.2029 |
| 06.01.4342 | (A)pmkPTT-TRE2CuO-rev | SEQ ID NO: 97 | SbfI + AscI | 06.01.2029 |
| 06.01.4343 | (A)pmkPTT-TRE2CuO(BGI)-VSVG | SEQ ID NO: 98 | SbfI + AscI | 06.01.2029 |
| 06.01.4344 | (A)pmkPTT-CAGGS(BGIC&R)-rtTA-CMV(BGI)-CymR-HygroR | SEQ ID NO: 99 | SbfI + AscI | 06.01.2029 |
| 06.01.4345 | (A)pmkBmYT-CMV(BGI)-gag/pol | SEQ ID NO: 96 | SbfI + AscI | 06.01.2037 |
| 06.01.4346 | (A)pmkBmYT-TRE2CuO-rev | SEQ ID NO: 97 | SbfI + AscI | 06.01.2037 |
| 06.01.4347 | (A)pmkBmYT-TRE2CuO(BGI)-VSVG | SEQ ID NO: 98 | SbfI + AscI | 06.01.2037 |
| 06.01.4348 | (A)pmkBmYT-CAGGS(BGIC&R)-rtTA-CMV(BGI)-CymR-HygroR | SEQ ID NO: 99 | SbfI + AscI | 06.01.2037 |
| 06.01.4349 | (A)pmkXtUT1-CMV(BGI)-gag/pol | SEQ ID NO: 96 | SbfI + AscI | 06.01.2052 |
| 06.01.4350 | (A)pmkXtUT1-TRE2CuO-rev | SEQ ID NO: 97 | SbfI + AscI | 06.01.2052 |
| 06.01.4351 | (A)pmkXtUT1-TRE2CuO(BGI)-VSVG | SEQ ID NO: 98 | SbfI + AscI | 06.01.2052 |
| 06.01.4352 | (A)pmkXtUT1-CAGGS(BGIC&R)-rtTA-CMV(BGI)-CymR-HygroR | SEQ ID NO: 99 | SbfI + AscI | 06.01.2052 |
| 06.01.4353 | (A)pmkHPBT-CMV(BGI)-gag/pol | SEQ ID NO: 96 | SbfI + AscI | 06.01.1917 |
| 06.01.4354 | (A)pmkHPBT-TRE2CuO-rev | SEQ ID NO: 97 | SbfI + AscI | 06.01.1917 |
| 06.01.4355 | (A)pmkHPBT-TRE2CuO(BGI)-VSVG | SEQ ID NO: 98 | SbfI + AscI | 06.01.1917 |
| 06.01.4356 | (A)pmkHPBT-CAGGS(BGIC&R)-rtTA-CMV(BGI)-CymR-HygroR | SEQ ID NO: 99 | SbfI + AscI | 06.01.1917 |
| 06.01.4357 | (A)pmkSBT3-CMV(BGI)-gag/pol | SEQ ID NO: 96 | SbfI + AscI | 06.01.1367 |
| 06.01.4358 | (A)pmkSBT3-TRE2CuO-rev | SEQ ID NO: 97 | SbfI + AscI | 06.01.1367 |
| 06.01.4359 | (A)pmkSBT3-TRE2CuO(BGI)-VSVG | SEQ ID NO: 98 | SbfI + AscI | 06.01.1367 |
| 06.01.4360 | (A)pmkSBT3-CAGGS(BGIC&R)-rtTA-CMV(BGI)-CymR-HygroR | SEQ ID NO: 99 | SbfI + AscI | 06.01.1367 |
| 06.01.4361 | (A)pmkTo1T-LVRSV-hPGK-Luciferase-ires-EGFP-PuroR | SEQ ID NO: 100 | SbfI + AscI | 06.01.1939 |
| 06.01.4362 | (A)pmkTo2T-LVRSV-hPGK-Luciferase-ires-EGFP-PuroR | SEQ ID NO: 100 | SbfI + AscI | 06.01.1946 |
| 06.01.4363 | (A)pmkFTP-LVRSV-hPGK-Luciferase-ires-EGFP-PuroR | SEQ ID NO: 100 | SbfI + AscI | 06.01.1952 |
| 06.01.4364 | (A)pmkMiT-LVRSV-hPGK-Luciferase-ires-EGFP-PuroR | SEQ ID NO: 100 | SbfI + AscI | 06.01.1957 |
| 06.01.4365 | (A)pmkHsmT-LVRSV-hPGK-Luciferase-ires-EGFP-PuroR | SEQ ID NO: 100 | SbfI + AscI | 06.01.1958 |
| 06.01.4366 | (A)pmkHelT-LVRSV-hPGK-Luciferase-ires-EGFP-PuroR | SEQ ID NO: 100 | SbfI + AscI | 06.01.1967 |
| 06.01.4367 | (A)pmkZBT-LVRSV-hPGK-Luciferase-ires-EGFP-PuroR | SEQ ID NO: 100 | SbfI + AscI | 06.01.1982 |
| 06.01.4368 | (A)pmkITT-LVRSV-hPGK-Luciferase-ires-EGFP-PuroR | SEQ ID NO: 100 | SbfI + AscI | 06.01.1985 |
| 06.01.4369 | (A)pmkSPINT-LVRSV-hPGK-Luciferase-ires-EGFP-PuroR | SEQ ID NO: 100 | SbfI + AscI | 06.01.2014 |
| 06.01.4370 | (A)pmkTcBT1-LVRSV-hPGK-Luciferase-ires-EGFP-PuroR | SEQ ID NO: 100 | SbfI + AscI | 06.01.2016 |
| 06.01.4371 | (A)pmkPTT-LVRSV-hPGK-Luciferase-ires-EGFP-PuroR | SEQ ID NO: 100 | SbfI + AscI | 06.01.2029 |
| 06.01.4372 | (A)pmkBmYT-LVRSV-hPGK-Luciferase-ires-EGFP-PuroR | SEQ ID NO: 100 | SbfI + AscI | 06.01.2037 |
| 06.01.4373 | (A)pmkXtUT1-LVRSV-hPGK-Luciferase-ires-EGFP-PuroR | SEQ ID NO: 100 | SbfI + AscI | 06.01.2052 |
| 06.01.4374 | (A)pmkHPBT-LVRSV-hPGK-Luciferase-ires-EGFP-PuroR | SEQ ID NO: 100 | SbfI + AscI | 06.01.1917 |
| 06.01.4375 | (A)pmkSBT3-LVRSV-hPGK-Luciferase-ires-EGFP-PuroR | SEQ ID NO: 100 | SbfI + AscI | 06.01.1367 |

### Example 2: Test of Gene Insertion Efficiency and Target Gene Activity in Cell Line Constructed Using Multi-Transposon System

For the production of most biological products, such as the production of recombinant proteins expressed in mammalian cells, the expression level per unit volume of a culture is positively correlated with the copy number of a nucleotide fragment of interest inserted in an engineered cell genome. Increasing the copy number of a nucleotide fragment of interest inserted is one of the most effective strategies to increase the expression level in a lentivirus producing cell line. However, there is an upper limit on the number of inserted copies for most transposon systems when one or more nucleotide fragments of interest are inserted. The inventors of the present application have demonstrated that using the multi-transposon system disclosed in the present disclosure can effectively increase the upper limit of the copy number of a nucleotide fragment of interest (gene of interest, GOI) inserted in a cell and significantly increase the expression level of a protein of interest.

hPGK-Luciferase-ires-EGFP-WPRE was used as a nucleotide fragment of interest to detect the effectiveness of a multi-transposon system in inserting a gene of interest: after being transfected into a mammalian cell, hPGK-Luciferase-ires-EGFP-WPRE would express luciferase and EGFP protein. In the cell, the luciferase activity is highly correlated with the luciferase protein expression level, and can be measured by luciferase assay. In addition, this nucleotide fragment of interest further contains WPRE sequence, which is used as a tag for quantifying the number of copies inserted in a host cell genome by qPCR. Three resistance genes, PuroR (puromycin resistance gene), HygroR (hygromycin resistance gene) and BSD (blasticidin resistance gene) were respectively ligated to this nucleotide fragment of interest for rapid screening of positive cell populations having the nucleotide fragment of interest stably inserted in the genome after transfection. The above-mentioned three nucleotide fragments of interest (PuroR(R)-hPGK-Luciferase-ires-EGFP-WPRE, HygroR(R)-hPGK-Luciferase-ires-EGFP-WPRE and BSD(R)-hPGK-Luciferase-ires-EGFP-WPRE) carrying different resistance genes were respectively cloned into different transposon plasmids containing the transposase recognition sequences terminal inverted repeats (TIRs) and used for the subsequent testing of the effectiveness of the multi-transposon system in inserting the nucleotide fragments of interest.

293T cells (ATCC, CRL3216) were firstly co-transfected with a first transposon plasmid containing the nucleotide fragment of interest (PuroR(R)-hPGK-Luciferase-ires-EGFP-WPRE) and a corresponding transposase expression plasmid, and a puromycin-resistant population was screened. The above-mentioned puromycin-resistant cells were then co-transfected with a second transposon plasmid that contained the nucleotide fragment of interest (HygroR(R)-hPGK-Luciferase-ires-EGFP-WPRE) and was different from the first transposon, and a corresponding transposase expression plasmid, and a puromycin and hygromycin double-resistant cell population was screened. Then, the luciferase expression levels were compared by detecting the luciferase activity in the double-resistant cell population, and the mean copy number of WPRE in the double-resistant cell population was detected by qPCR. The numbers of the first and second transposon plasmids and the corresponding transposase plasmids, the luciferase activity measured, and the copy number of WPRE measured by qPCR are as shown in Table 4. Specific experimental steps are briefly described below.

The experimental process of stably inserting a nucleotide fragment of interest in the genome of 293T cells by a transposon system is briefly described as follows. 293T cells (ATCC, CRL3216) were cultured at 37° C. and 5% CO₂ in a DMEM complete medium (DMEM (Sigam, D6429)) supplemented with 10% FBS (ExCell, 11H 116). The 293T cells were seeded in a 6-well plate (Corning, 3516) at 8E+05 cells/well. After 24 hours of culture, a transfection reagent was prepared according to the calcium phosphate transfection method as described in “MolecμLar Cloning: A Laboratory Manual (Fourth edition) Chapter 15, Michael R. Green, Cold Spring Harbor Laboratory Press, 2012”, and 200 μL of the transfection reagent containing 0.12 mol/L calcium chloride, 1×HEPES buffer and 5.5 μg of the total plasmids was added to each well. The co-transfection of the transposon plasmid and the transposase plasmid was performed at a molar ratio of 5:1 using calcium phosphate, wherein the plasmid numbers of the transposon and transposase are as shown in Table 4. After 24 hours of transfection, the medium was replaced with a fresh DMEM complete medium containing 2.5 μg/ml puromycin (Aladdin P113126). Screening was performed under antibiotic pressure for at least 3 consecutive passages until the cells stably grew. Then, the cells constructed above were co-transfected with the second transposon plasmid and the corresponding transposase plasmid thereof according to the same experimental method and cultured in a DMEM complete medium containing 200 μg/ml hygromycin (Shenggong A600230-0001) for at least three passages.

The experimental process of detecting the luciferase activity in each cell line using a luciferase detection kit is briefly described as follows. Each cell line was seeded in a 96-well plate (Corning 3916) at 1E+04 cells/well, with duplicate wells for each cell line. After 48 hours of culture, the relative luciferase unit (RLU) of each well was detected using a Steady-Glo luciferase assay system (Promega, E2610) kit according to the instructions for use (Promega, FB037), wherein the detection instrument was fluorescence microplate reader (Perkin Elmer VictorV).

The experimental process of measuring the copy number of WPRE in each cell line by qPCR is briefly described as follows. 1.0E+06 cells of each of the above cell lines were collected, and the genomic gDNA was extracted according to the instructions for use of a genomic DNA purification kit (TIANGEN, DP304-03). The purified gDNA was adjusted to 50 ng/μl using the elution buffer in the kit. Plasmid 06.01.2141 was diluted with deionized water to 47.9 ng/μl (corresponding to 5.0E+09 copies/μL) as a standard for WPRE, and this standard was further diluted to 8.0E+06 copies/μL. This standard (8E+06 copies/μL) was then serially diluted in two-fold to 1.5625E+04 copies/μL, and these 9 serially diluted samples were used as standard curve samples for the WPRE sequence. 1 μL of the gDNA sample of each cell line and the qPCR standard curve sample were added to a Taqman probe qPCR mix, and made up to 20 μL with water, wherein the qPCR mix contained 10 μL of NovoStart Probe qPCR SuperMix, 0.4 μL of ROX I dye (Novoprotein Scientific Inc., E091-01A), 0.4 μL of WPRE-taqman-F forward primer and WPRE-taqman-R reverse primer at a concentration of 10 μM, and 0.4 μL of WPRE-Probe Taqman probe (synthesized by General Biosystems (Anhui) Co., Ltd.) at a concentration of 10 μM, and the specific information of the primers and probe are as shown in Table 2 above, where the forward and reverse primers and the probe for WPRE are SEQ ID NO: 130, SEQ ID NO: 131 and SEQ ID NO: 132, respectively. PCR reaction was performed using an ABI 7900 real-time PCR instrument according to AQ procedure and the following steps: 95° C. for 5 min, 40 cycles of 95° C. for 30 seconds-60° C. for 30 seconds-72° C. for 30 seconds, 60° C. for 30 seconds. The copy concentration (copies/μL) of the WPRE fragment in each sample was calculated on the basis of the standard curve and the CT value of the sample, and then the number of copies containing the WPRE fragment per cell (copies/cell) was calculated according to 6 μg of genomic DNA contained in each cell.

Table 4 summarizes the luciferase activity RLU and the copy number of inserted WPRE in the double-resistant cell lines constructed using various transposon combinations. The mean luciferase activity and the mean copy number of inserted WPRE in the cell line constructed only using a single transposon are 2.67E+05 RLU and 3.12 copies (WPRE)/cell, respectively. The luciferase activity and the copy number of inserted WPRE in the optimal cell that can be obtained are 5.11E+05 RLU and 5.14 copies (WPRE)/cell, respectively. The mean luciferase activity and the mean copy number of inserted WPRE in the cell line constructed using the dual-transposon method described in the present disclosure increase to 4.45E+05 RLU and 5.83 copies (WPRE)/cell, with an increase of 66.62% and 87.10%. The luciferase activity and the copy number of inserted WPRE in the optimal cell that can be obtained are 8.12E+05 RLU and 12.04 copies (WPRE)/cell, respectively. However, the luciferase activity (2.36E+05 RLU) and the copy number of inserted WPRE (2.80 copies (WPRE)/cell) in the cell line constructed by two transient transfections and resistance screening using the same transposon system are both slightly inferior to those in the cell line constructed by one transient transfection using the same transposon system. There are significant increases in both luciferase activity and the copy number of inserted WPRE in the cell line constructed using the dual-transposon system over the cell line constructed using either of the two single transposon systems. In addition, the Tol1, Tol2, ZB, Intruder, TcBuster, Yabusame-1, Uribo2, Sleeping Beauty and piggyBac transposon systems have higher activity. The mean luciferase activity (5.47E+05 RLU) and the mean copy number of inserted WPRE (7.53 copies (WPRE)/cell) in the cell lines (a total of 36 cell lines) constructed using the combination of the above transposon systems are respectively 71.36% and 92.08% higher than the mean luciferase activity (3.19E+05 RLU) and the mean copy number of inserted of WPRE (3.92 copies/cell) in the cell lines constructed using any of these 9 transposon systems alone.

The 13 cell lines in which the piggyBac transposon system (06.01.1757 and 06.01.2429) was used as the second transposon system in the above experiment were selected and the Sleeping Beauty transposon system (06.01.1807 and 06.01.3335) was used as the third transposon system to insert the nucleotide fragment of interest (BSD(R)-hPGK-Luciferase-ires-EGFP-WPRE) into the genome of the above-mentioned cells (as shown in Table 5). The experimental operations of cell culture, plasmid transfection, antibiotic selection, luciferase assay and qPCR quantification of the copy number of WPRE are described above. BSD positive cells were screened using 3 μg/mL blasticidin S (SHANGHAI MAOKANG BIOTECHNOLOGY, Cat. No. #MS0007). The results of the luciferin activity and the copy number of WPRE inserted in the genome are described in Table 5. The mean luciferase activity and the mean copy number of inserted WPRE in the cell line constructed using the three-transposon system are 7.22E+05 RLU and 9.46 copies (WPRE)/cell, respectively. The luciferase activity and the copy number of inserted WPRE in the optimal cell line are 9.52E+05 and 12.76 copies (WPRE)/cell, respectively, which are respectively 57.72% and 61.75% higher than the mean luciferase activity (6.04E+05) and the mean copy number of inserted WPRE (7.89 copies (WPRE)/cell) in the cell line constructed using the SB and PB dual-transposon system.

TABLE 4
Test of the copy number of inserted gene and target gene activity
in cell line constructed using dual-transposon system

| First transposon system | | | Second transposon system | | | | Copies/cell |
|---|---|---|---|---|---|---|---|
| Transposon | Transposase | Antibiotic | Transposon | Transposase | Antibiotic | RLU | (WPRE) |
| 06.01.2141 | 06.01.1494 | Puromycin | n/a | n/a | n/a | 3.30E+05 | 3.54 |
| 06.01.2143 | 06.01.1504 | Puromycin | n/a | n/a | n/a | 2.55E+05 | 3.64 |
| 06.01.2148 | 06.01.1527 | Puromycin | n/a | n/a | n/a | 1.74E+05 | 1.86 |
| 06.01.2170 | 06.01.1535 | Puromycin | n/a | n/a | n/a | 2.25E+05 | 2.09 |
| 06.01.2206 | 06.01.1538 | Puromycin | n/a | n/a | n/a | 1.56E+05 | 1.53 |
| 06.01.2218 | 06.01.1579 | Puromycin | n/a | n/a | n/a | 1.40E+05 | 1.34 |
| 06.01.2229 | 06.01.1573 | Puromycin | n/a | n/a | n/a | 3.38E+05 | 3.92 |
| 06.01.2250 | 06.01.1582 | Puromycin | n/a | n/a | n/a | 3.14E+05 | 3.73 |
| 06.01.2259 | 06.01.1596 | Puromycin | n/a | n/a | n/a | 2.64E+05 | 3.02 |
| 06.01.2263 | 06.01.1614 | Puromycin | n/a | n/a | n/a | 3.48E+05 | 5.14 |
| 06.01.2263 | 06.01.1681 | Puromycin | n/a | n/a | n/a | 3.65E+05 | 3.06 |
| 06.01.2263 | 06.01.1696 | Puromycin | n/a | n/a | n/a | 3.43E+05 | 4.13 |
| 06.01.2263 | 06.01.1703 | Puromycin | n/a | n/a | n/a | 3.37E+05 | 3.33 |
| 06.01.2263 | 06.01.1705 | Puromycin | n/a | n/a | n/a | 3.21E+05 | 3.34 |
| 06.01.2273 | 06.01.1614 | Puromycin | n/a | n/a | n/a | 3.15E+05 | 3.77 |
| 06.01.2273 | 06.01.1681 | Puromycin | n/a | n/a | n/a | 3.11E+05 | 3.88 |
| 06.01.2273 | 06.01.1696 | Puromycin | n/a | n/a | n/a | 3.08E+05 | 4.02 |
| 06.01.2273 | 06.01.1703 | Puromycin | n/a | n/a | n/a | 3.03E+05 | 3.98 |
| 06.01.2273 | 06.01.1705 | Puromycin | n/a | n/a | n/a | 3.66E+05 | 3.03 |
| 06.01.2277 | 06.01.1679 | Puromycin | n/a | n/a | n/a | 1.73E+05 | 1.90 |
| 06.01.2286 | 06.01.1687 | Puromycin | n/a | n/a | n/a | 2.48E+05 | 4.34 |
| 06.01.2286 | 06.01.1740 | Puromycin | n/a | n/a | n/a | 4.03E+05 | 4.95 |
| 06.01.2286 | 06.01.1517 | Puromycin | n/a | n/a | n/a | 2.10E+05 | 1.53 |
| 06.01.2286 | 06.01.1778 | Puromycin | n/a | n/a | n/a | 2.24E+05 | 3.98 |
| 06.01.2286 | 06.01.1795 | Puromycin | n/a | n/a | n/a | 4.10E+05 | 5.22 |
| 06.01.2289 | 06.01.1770 | Puromycin | n/a | n/a | n/a | 3.14E+05 | 4.62 |
| 06.01.2289 | 06.01.1790 | Puromycin | n/a | n/a | n/a | 3.80E+05 | 4.76 |
| 06.01.2289 | 06.01.1850 | Puromycin | n/a | n/a | n/a | 4.08E+05 | 3.33 |
| 06.01.2289 | 06.01.1862 | Puromycin | n/a | n/a | n/a | 4.05E+05 | 3.39 |
| 06.01.2289 | 06.01.1872 | Puromycin | n/a | n/a | n/a | 2.35E+05 | 2.88 |
| 06.01.2289 | 06.01.1884 | Puromycin | n/a | n/a | n/a | 3.21E+05 | 4.22 |
| 06.01.2305 | 06.01.1770 | Puromycin | n/a | n/a | n/a | 3.91E+05 | 3.24 |
| 06.01.2305 | 06.01.1790 | Puromycin | n/a | n/a | n/a | 4.72E+05 | 4.47 |
| 06.01.2305 | 06.01.1850 | Puromycin | n/a | n/a | n/a | 4.51E+05 | 4.76 |
| 06.01.2305 | 06.01.1862 | Puromycin | n/a | n/a | n/a | 4.14E+05 | 3.89 |
| 06.01.2305 | 06.01.1872 | Puromycin | n/a | n/a | n/a | 2.63E+05 | 3.31 |
| 06.01.2305 | 06.01.1884 | Puromycin | n/a | n/a | n/a | 3.45E+05 | 4.01 |
| 06.01.2331 | 06.01.1757 | Puromycin | n/a | n/a | n/a | 2.13E+05 | 2.03 |
| 06.01.2331 | 06.01.1581 | Puromycin | n/a | n/a | n/a | 1.85E+05 | 1.97 |
| 06.01.2331 | 06.01.1892 | Puromycin | n/a | n/a | n/a | 3.62E+05 | 4.06 |
| 06.01.2331 | 06.01.1903 | Puromycin | n/a | n/a | n/a | 2.89E+05 | 3.79 |
| 06.01.2336 | 06.01.1757 | Puromycin | n/a | n/a | n/a | 2.37E+05 | 1.98 |
| 06.01.2336 | 06.01.1581 | Puromycin | n/a | n/a | n/a | 1.78E+05 | 2.00 |
| 06.01.2336 | 06.01.1892 | Puromycin | n/a | n/a | n/a | 4.00E+05 | 4.24 |
| 06.01.2336 | 06.01.1903 | Puromycin | n/a | n/a | n/a | 3.06E+05 | 3.89 |
| 06.01.2343 | 06.01.1807 | Puromycin | n/a | n/a | n/a | 5.11E+05 | 4.35 |
| 06.01.2343 | 06.01.1941 | Puromycin | n/a | n/a | n/a | 5.83E+05 | 4.56 |
| 06.01.2327 | 06.01.1807 | Puromycin | n/a | n/a | n/a | 4.90E+05 | 4.22 |
| 06.01.2327 | 06.01.1941 | Puromycin | n/a | n/a | n/a | 5.79E+00 | 4.67 |
| 06.01.2141 | 06.01.1494 | Puromycin | 06.01.2347 | 06.01.1504 | Hygromycin | 5.55E+05 | 7.85 |
| 06.01.2141 | 06.01.1494 | Puromycin | 06.01.3321 | 06.01.1527 | Hygromycin | 4.04E+05 | 4.54 |
| 06.01.2141 | 06.01.1494 | Puromycin | 06.01.3331 | 06.01.1535 | Hygromycin | 4.44E+05 | 4.83 |
| 06.01.2141 | 06.01.1494 | Puromycin | 06.01.3372 | 06.01.1538 | Hygromycin | 4.37E+05 | 4.02 |
| 06.01.2141 | 06.01.1494 | Puromycin | 06.01.2358 | 06.01.1579 | Hygromycin | 3.98E+05 | 4.43 |
| 06.01.2141 | 06.01.1494 | Puromycin | 06.01.3428 | 06.01.1573 | Hygromycin | 5.13E+05 | 7.44 |
| 06.01.2141 | 06.01.1494 | Puromycin | 06.01.3455 | 06.01.1582 | Hygromycin | 5.18E+05 | 6.59 |
| 06.01.2141 | 06.01.1494 | Puromycin | 06.01.3457 | 06.01.1596 | Hygromycin | 4.64E+05 | 5.37 |
| 06.01.2141 | 06.01.1494 | Puromycin | 06.01.2360 | 06.01.1614 | Hygromycin | 5.74E+05 | 7.15 |
| 06.01.2141 | 06.01.1494 | Puromycin | 06.01.2879 | 06.01.1679 | Hygromycin | 4.08E+05 | 4.60 |
| 06.01.2141 | 06.01.1494 | Puromycin | 06.01.2370 | 06.01.1687 | Hygromycin | 5.23E+05 | 9.58 |
| 06.01.2141 | 06.01.1494 | Puromycin | 06.01.2379 | 06.01.1770 | Hygromycin | 4.64E+05 | 6.51 |
| 06.01.2141 | 06.01.1494 | Puromycin | 06.01.2429 | 06.01.1757 | Hygromycin | 4.83E+05 | 5.04 |
| 06.01.2141 | 06.01.1494 | Puromycin | 06.01.2433 | 06.01.1807 | Hygromycin | 7.23E+05 | 8.65 |
| 06.01.2143 | 06.01.1504 | Puromycin | 06.01.3321 | 06.01.1527 | Hygromycin | 3.52E+05 | 5.23 |
| 06.01.2143 | 06.01.1504 | Puromycin | 06.01.3331 | 06.01.1535 | Hygromycin | 3.78E+05 | 4.98 |
| 06.01.2143 | 06.01.1504 | Puromycin | 06.01.3372 | 06.01.1538 | Hygromycin | 3.27E+05 | 4.30 |
| 06.01.2143 | 06.01.1504 | Puromycin | 06.01.2358 | 06.01.1579 | Hygromycin | 3.60E+05 | 4.40 |
| 06.01.2143 | 06.01.1504 | Puromycin | 06.01.3428 | 06.01.1573 | Hygromycin | 5.15E+05 | 6.68 |
| 06.01.2143 | 06.01.1504 | Puromycin | 06.01.3455 | 06.01.1582 | Hygromycin | 4.72E+05 | 8.15 |
| 06.01.2143 | 06.01.1504 | Puromycin | 06.01.3457 | 06.01.1596 | Hygromycin | 4.10E+05 | 5.52 |
| 06.01.2143 | 06.01.1504 | Puromycin | 06.01.2360 | 06.01.1614 | Hygromycin | 4.93E+05 | 8.45 |
| 06.01.2143 | 06.01.1504 | Puromycin | 06.01.2879 | 06.01.1679 | Hygromycin | 3.46E+05 | 6.11 |
| 06.01.2143 | 06.01.1504 | Puromycin | 06.01.2370 | 06.01.1687 | Hygromycin | 4.49E+05 | 8.33 |
| 06.01.2143 | 06.01.1504 | Puromycin | 06.01.2379 | 06.01.1770 | Hygromycin | 4.13E+05 | 6.65 |
| 06.01.2143 | 06.01.1504 | Puromycin | 06.01.2429 | 06.01.1757 | Hygromycin | 4.15E+05 | 6.74 |
| 06.01.2143 | 06.01.1504 | Puromycin | 06.01.2433 | 06.01.1807 | Hygromycin | 5.76E+05 | 7.30 |
| 06.01.2148 | 06.01.1527 | Puromycin | 06.01.3331 | 06.01.1535 | Hygromycin | 2.92E+05 | 3.68 |
| 06.01.2148 | 06.01.1527 | Puromycin | 06.01.3372 | 06.01.1538 | Hygromycin | 2.66E+05 | 3.11 |
| 06.01.2148 | 06.01.1527 | Puromycin | 06.01.2358 | 06.01.1579 | Hygromycin | 2.84E+05 | 2.84 |
| 06.01.2148 | 06.01.1527 | Puromycin | 06.01.3428 | 06.01.1573 | Hygromycin | 4.23E+05 | 5.45 |
| 06.01.2148 | 06.01.1527 | Puromycin | 06.01.3455 | 06.01.1582 | Hygromycin | 3.69E+05 | 3.79 |
| 06.01.2148 | 06.01.1527 | Puromycin | 06.01.3457 | 06.01.1596 | Hygromycin | 3.48E+05 | 3.59 |
| 06.01.2148 | 06.01.1527 | Puromycin | 06.01.2360 | 06.01.1614 | Hygromycin | 4.38E+05 | 8.07 |
| 06.01.2148 | 06.01.1527 | Puromycin | 06.01.2879 | 06.01.1679 | Hygromycin | 3.07E+05 | 3.44 |
| 06.01.2148 | 06.01.1527 | Puromycin | 06.01.2370 | 06.01.1687 | Hygromycin | 3.89E+05 | 8.67 |
| 06.01.2148 | 06.01.1527 | Puromycin | 06.01.2379 | 06.01.1770 | Hygromycin | 4.49E+05 | 4.78 |
| 06.01.2148 | 06.01.1527 | Puromycin | 06.01.2429 | 06.01.1757 | Hygromycin | 3.42E+05 | 3.63 |
| 06.01.2148 | 06.01.1527 | Puromycin | 06.01.2433 | 06.01.1807 | Hygromycin | 4.57E+05 | 7.30 |
| 06.01.2170 | 06.01.1535 | Puromycin | 06.01.3372 | 06.01.1538 | Hygromycin | 3.05E+05 | 2.99 |
| 06.01.2170 | 06.01.1535 | Puromycin | 06.01.2358 | 06.01.1579 | Hygromycin | 2.98E+05 | 2.86 |
| 06.01.2170 | 06.01.1535 | Puromycin | 06.01.3428 | 06.01.1573 | Hygromycin | 5.24E+05 | 4.72 |
| 06.01.2170 | 06.01.1535 | Puromycin | 06.01.3455 | 06.01.1582 | Hygromycin | 4.06E+05 | 5.72 |
| 06.01.2170 | 06.01.1535 | Puromycin | 06.01.3457 | 06.01.1596 | Hygromycin | 4.01E+05 | 4.44 |
| 06.01.2170 | 06.01.1535 | Puromycin | 06.01.2360 | 06.01.1614 | Hygromycin | 4.85E+05 | 6.66 |
| 06.01.2170 | 06.01.1535 | Puromycin | 06.01.2879 | 06.01.1679 | Hygromycin | 3.06E+05 | 3.21 |
| 06.01.2170 | 06.01.1535 | Puromycin | 06.01.2370 | 06.01.1687 | Hygromycin | 3.97E+05 | 5.01 |
| 06.01.2170 | 06.01.1535 | Puromycin | 06.01.2379 | 06.01.1770 | Hygromycin | 4.00E+05 | 6.11 |
| 06.01.2170 | 06.01.1535 | Puromycin | 06.01.2429 | 06.01.1757 | Hygromycin | 3.93E+05 | 3.10 |
| 06.01.2170 | 06.01.1535 | Puromycin | 06.01.2433 | 06.01.1807 | Hygromycin | 7.56E+05 | 6.05 |
| 06.01.2206 | 06.01.1538 | Puromycin | 06.01.2358 | 06.01.1579 | Hygromycin | 2.61E+05 | 2.56 |
| 06.01.2206 | 06.01.1538 | Puromycin | 06.01.3428 | 06.01.1573 | Hygromycin | 3.94E+05 | 5.09 |
| 06.01.2206 | 06.01.1538 | Puromycin | 06.01.3455 | 06.01.1582 | Hygromycin | 4.00E+05 | 5.73 |
| 06.01.2206 | 06.01.1538 | Puromycin | 06.01.3457 | 06.01.1596 | Hygromycin | 3.32E+05 | 3.64 |
| 06.01.2206 | 06.01.1538 | Puromycin | 06.01.2360 | 06.01.1614 | Hygromycin | 4.64E+05 | 7.72 |
| 06.01.2206 | 06.01.1538 | Puromycin | 06.01.2879 | 06.01.1679 | Hygromycin | 2.36E+05 | 2.99 |
| 06.01.2206 | 06.01.1538 | Puromycin | 06.01.2370 | 06.01.1687 | Hygromycin | 3.70E+05 | 5.98 |
| 06.01.2206 | 06.01.1538 | Puromycin | 06.01.2379 | 06.01.1770 | Hygromycin | 3.48E+05 | 4.12 |
| 06.01.2206 | 06.01.1538 | Puromycin | 06.01.2429 | 06.01.1757 | Hygromycin | 2.65E+05 | 2.74 |
| 06.01.2206 | 06.01.1538 | Puromycin | 06.01.2433 | 06.01.1807 | Hygromycin | 5.44E+05 | 7.09 |
| 06.01.2218 | 06.01.1579 | Puromycin | 06.01.3428 | 06.01.1573 | Hygromycin | 4.11E+05 | 5.21 |
| 06.01.2218 | 06.01.1579 | Puromycin | 06.01.3455 | 06.01.1582 | Hygromycin | 2.98E+05 | 4.04 |
| 06.01.2218 | 06.01.1579 | Puromycin | 06.01.3457 | 06.01.1596 | Hygromycin | 3.39E+05 | 3.06 |
| 06.01.2218 | 06.01.1579 | Puromycin | 06.01.2360 | 06.01.1614 | Hygromycin | 2.86E+05 | 4.47 |
| 06.01.2218 | 06.01.1579 | Puromycin | 06.01.2879 | 06.01.1679 | Hygromycin | 2.84E+05 | 3.28 |
| 06.01.2218 | 06.01.1579 | Puromycin | 06.01.2370 | 06.01.1687 | Hygromycin | 2.87E+05 | 5.07 |
| 06.01.2218 | 06.01.1579 | Puromycin | 06.01.2379 | 06.01.1770 | Hygromycin | 3.57E+05 | 5.20 |
| 06.01.2218 | 06.01.1579 | Puromycin | 06.01.2429 | 06.01.1757 | Hygromycin | 3.20E+05 | 2.74 |
| 06.01.2218 | 06.01.1579 | Puromycin | 06.01.2433 | 06.01.1807 | Hygromycin | 4.45E+05 | 4.54 |
| 06.01.2229 | 06.01.1573 | Puromycin | 06.01.3455 | 06.01.1582 | Hygromycin | 5.55E+05 | 7.30 |
| 06.01.2229 | 06.01.1573 | Puromycin | 06.01.3457 | 06.01.1596 | Hygromycin | 4.92E+05 | 6.58 |
| 06.01.2229 | 06.01.1573 | Puromycin | 06.01.2360 | 06.01.1614 | Hygromycin | 6.50E+05 | 12.04 |
| 06.01.2229 | 06.01.1573 | Puromycin | 06.01.2879 | 06.01.1679 | Hygromycin | 4.31E+05 | 7.15 |
| 06.01.2229 | 06.01.1573 | Puromycin | 06.01.2370 | 06.01.1687 | Hygromycin | 5.02E+05 | 6.60 |
| 06.01.2229 | 06.01.1573 | Puromycin | 06.01.2379 | 06.01.1770 | Hygromycin | 6.00E+05 | 8.58 |
| 06.01.2229 | 06.01.1573 | Puromycin | 06.01.2429 | 06.01.1757 | Hygromycin | 4.54E+05 | 5.36 |
| 06.01.2229 | 06.01.1573 | Puromycin | 06.01.2433 | 06.01.1807 | Hygromycin | 6.17E+05 | 6.27 |
| 06.01.2250 | 06.01.1582 | Puromycin | 06.01.3457 | 06.01.1596 | Hygromycin | 4.20E+05 | 5.85 |
| 06.01.2250 | 06.01.1582 | Puromycin | 06.01.2360 | 06.01.1614 | Hygromycin | 5.70E+05 | 7.45 |
| 06.01.2250 | 06.01.1582 | Puromycin | 06.01.2879 | 06.01.1679 | Hygromycin | 4.24E+05 | 5.40 |
| 06.01.2250 | 06.01.1582 | Puromycin | 06.01.2370 | 06.01.1687 | Hygromycin | 5.04E+05 | 5.94 |
| 06.01.2250 | 06.01.1582 | Puromycin | 06.01.2379 | 06.01.1770 | Hygromycin | 5.08E+05 | 7.12 |
| 06.01.2250 | 06.01.1582 | Puromycin | 06.01.2429 | 06.01.1757 | Hygromycin | 4.43E+05 | 5.25 |
| 06.01.2250 | 06.01.1582 | Puromycin | 06.01.2433 | 06.01.1807 | Hygromycin | 7.50E+05 | 7.09 |
| 06.01.2259 | 06.01.1596 | Puromycin | 06.01.2360 | 06.01.1614 | Hygromycin | 4.71E+05 | 6.29 |
| 06.01.2259 | 06.01.1596 | Puromycin | 06.01.2879 | 06.01.1679 | Hygromycin | 3.60E+05 | 4.02 |
| 06.01.2259 | 06.01.1596 | Puromycin | 06.01.2370 | 06.01.1687 | Hygromycin | 4.40E+05 | 6.25 |
| 06.01.2259 | 06.01.1596 | Puromycin | 06.01.2379 | 06.01.1770 | Hygromycin | 5.06E+05 | 6.57 |
| 06.01.2259 | 06.01.1596 | Puromycin | 06.01.2429 | 06.01.1757 | Hygromycin | 3.96E+05 | 4.37 |
| 06.01.2259 | 06.01.1596 | Puromycin | 06.01.2433 | 06.01.1807 | Hygromycin | 6.17E+05 | 5.96 |
| 06.01.2263 | 06.01.1614 | Puromycin | 06.01.2879 | 06.01.1679 | Hygromycin | 4.40E+05 | 8.45 |
| 06.01.2263 | 06.01.1614 | Puromycin | 06.01.2370 | 06.01.1687 | Hygromycin | 5.35E+05 | 8.39 |
| 06.01.2263 | 06.01.1614 | Puromycin | 06.01.2379 | 06.01.1770 | Hygromycin | 5.56E+05 | 8.99 |
| 06.01.2263 | 06.01.1614 | Puromycin | 06.01.2429 | 06.01.1757 | Hygromycin | 5.36E+05 | 6.72 |
| 06.01.2263 | 06.01.1614 | Puromycin | 06.01.2433 | 06.01.1807 | Hygromycin | 7.21E+05 | 10.60 |
| 06.01.2277 | 06.01.1679 | Puromycin | 06.01.2370 | 06.01.1687 | Hygromycin | 3.36E+05 | 5.48 |
| 06.01.2277 | 06.01.1679 | Puromycin | 06.01.2379 | 06.01.1770 | Hygromycin | 3.72E+05 | 6.23 |
| 06.01.2277 | 06.01.1679 | Puromycin | 06.01.2429 | 06.01.1757 | Hygromycin | 3.47E+05 | 3.83 |
| 06.01.2277 | 06.01.1679 | Puromycin | 06.01.2433 | 06.01.1807 | Hygromycin | 5.59E+05 | 5.56 |
| 06.01.2286 | 06.01.1687 | Puromycin | 06.01.2379 | 06.01.1770 | Hygromycin | 5.12E+05 | 8.13 |
| 06.01.2286 | 06.01.1687 | Puromycin | 06.01.2429 | 06.01.1757 | Hygromycin | 3.91E+05 | 6.38 |
| 06.01.2286 | 06.01.1687 | Puromycin | 06.01.2433 | 06.01.1807 | Hygromycin | 6.30E+05 | 8.29 |
| 06.01.2305 | 06.01.1770 | Puromycin | 06.01.2429 | 06.01.1757 | Hygromycin | 4.84E+05 | 7.06 |
| 06.01.2305 | 06.01.1770 | Puromycin | 06.01.2433 | 06.01.1807 | Hygromycin | 8.12E+05 | 8.10 |
| 06.01.2331 | 06.01.1757 | Puromycin | 06.01.2433 | 06.01.1807 | Hygromycin | 6.72E+05 | 8.44 |
| 06.01.2141 | 06.01.1494 | Puromycin | 06.01.3233 | 06.01.1494 | Hygromycin | 2.57E+05 | 3.37 |
| 06.01.2143 | 06.01.1504 | Puromycin | 06.01.2347 | 06.01.1504 | Hygromycin | 2.29E+05 | 3.76 |
| 06.01.2148 | 06.01.1527 | Puromycin | 06.01.3321 | 06.01.1527 | Hygromycin | 2.26E+05 | 1.83 |
| 06.01.2170 | 06.01.1535 | Puromycin | 06.01.3331 | 06.01.1535 | Hygromycin | 2.68E+05 | 3.41 |
| 06.01.2206 | 06.01.1538 | Puromycin | 06.01.3372 | 06.01.1538 | Hygromycin | 1.48E+05 | 1.18 |
| 06.01.2218 | 06.01.1579 | Puromycin | 06.01.2358 | 06.01.1579 | Hygromycin | 1.04E+05 | 1.36 |
| 06.01.2229 | 06.01.1573 | Puromycin | 06.01.3428 | 06.01.1573 | Hygromycin | 3.05E+05 | 2.08 |
| 06.01.2250 | 06.01.1582 | Puromycin | 06.01.3455 | 06.01.1582 | Hygromycin | 2.12E+05 | 3.51 |
| 06.01.2259 | 06.01.1596 | Puromycin | 06.01.3457 | 06.01.1596 | Hygromycin | 2.06E+05 | 1.44 |
| 06.01.2263 | 06.01.1614 | Puromycin | 06.01.2360 | 06.01.1614 | Hygromycin | 2.60E+05 | 5.55 |
| 06.01.2277 | 06.01.1679 | Puromycin | 06.01.2879 | 06.01.1679 | Hygromycin | 5.80E+04 | 1.46 |
| 06.01.2286 | 06.01.1687 | Puromycin | 06.01.2370 | 06.01.1687 | Hygromycin | 2.34E+05 | 2.92 |
| 06.01.2289 | 06.01.1770 | Puromycin | 06.01.2379 | 06.01.1770 | Hygromycin | 2.79E+05 | 4.34 |
| 06.01.2331 | 06.01.1757 | Puromycin | 06.01.2429 | 06.01.1757 | Hygromycin | 1.72E+05 | 1.92 |
| 06.01.2343 | 06.01.1807 | Puromycin | 06.01.2433 | 06.01.1807 | Hygromycin | 5.79E+05 | 3.92 |

TABLE 5
Test of the copy number of inserted gene and target gene activity in cell line constructed using three-transposon system

| First transposon system | | | Second transposon system (PB) | | | Third transposon system (SB) | | | | Copies/cell |
|---|---|---|---|---|---|---|---|---|---|---|
| Transposon | Transposase | Antibiotic | Transposon | Transposase | Antibiotic | Transposon | Transposase | Antibiotic | RLU | (WPRE) |
| 06.01.2141 | 06.01.1494 | Puromycin | 06.01.2429 | 06.01.1757 | Hygromycin | 06.01.3335 | 06.01.1807 | Blasticidin | 8.28E+05 | 11.07 |
| 06.01.2143 | 06.01.1504 | Puromycin | 06.01.2429 | 06.01.1757 | Hygromycin | 06.01.3335 | 06.01.1807 | Blasticidin | 6.85E+05 | 12.76 |
| 06.01.2148 | 06.01.1527 | Puromycin | 06.01.2429 | 06.01.1757 | Hygromycin | 06.01.3335 | 06.01.1807 | Blasticidin | 5.77E+05 | 7.29 |
| 06.01.2170 | 06.01.1535 | Puromycin | 06.01.2429 | 06.01.1757 | Hygromycin | 06.01.3335 | 06.01.1807 | Blasticidin | 6.12E+05 | 7.17 |
| 06.01.2206 | 06.01.1538 | Puromycin | 06.01.2429 | 06.01.1757 | Hygromycin | 06.01.3335 | 06.01.1807 | Blasticidin | 5.80E+05 | 8.85 |
| 06.01.2218 | 06.01.1579 | Puromycin | 06.01.2429 | 06.01.1757 | Hygromycin | 06.01.3335 | 06.01.1807 | Blasticidin | 5.75E+05 | 6.57 |
| 06.01.2229 | 06.01.1573 | Puromycin | 06.01.2429 | 06.01.1757 | Hygromycin | 06.01.3335 | 06.01.1807 | Blasticidin | 7.89E+05 | 10.26 |
| 06.01.2250 | 06.01.1582 | Puromycin | 06.01.2429 | 06.01.1757 | Hygromycin | 06.01.3335 | 06.01.1807 | Blasticidin | 7.56E+05 | 8.75 |
| 06.01.2259 | 06.01.1596 | Puromycin | 06.01.2429 | 06.01.1757 | Hygromycin | 06.01.3335 | 06.01.1807 | Blasticidin | 7.48E+05 | 7.65 |
| 06.01.2263 | 06.01.1614 | Puromycin | 06.01.2429 | 06.01.1757 | Hygromycin | 06.01.3335 | 06.01.1807 | Blasticidin | 9.52E+05 | 9.60 |
| 06.01.2277 | 06.01.1679 | Puromycin | 06.01.2429 | 06.01.1757 | Hygromycin | 06.01.3335 | 06.01.1807 | Blasticidin | 6.45E+05 | 8.06 |
| 06.01.2286 | 06.01.1687 | Puromycin | 06.01.2429 | 06.01.1757 | Hygromycin | 06.01.3335 | 06.01.1807 | Blasticidin | 8.25E+05 | 12.19 |
| 06.01.2305 | 06.01.1770 | Puromycin | 06.01.2429 | 06.01.1757 | Hygromycin | 06.01.3335 | 06.01.1807 | Blasticidin | 9.32E+05 | 12.59 |
| 06.01.2331 | 06.01.1757 | Puromycin | 06.01.2433 | 06.01.1807 | Hygromycin | n/a | n/a | n/a | 6.04E+05 | 7.89 |

### Example 3: Construction of Stable Lentivirus Producing Cell Line Using Multi-Transposon System

Construction of a complex cell line typically requires multiple modification and screening steps on a host cell, including insertion of multiple nucleotide fragments of interest, adjustment of the insertion ratio of the multiple inserted nucleotide fragments of interest, and subsequent modification of previously modified cells. The inventors of the present application have demonstrated that the method described in the present disclosure is capable of effectively inserting multiple nucleotide fragments of interest in a host cell genome at a significantly higher copy number and of effectively adjusting the insertion ratio of the multiple inserted nucleotide fragments of interest. The construction of a stable virus vector producing cell line typically requires the insertion of multiple nucleotide fragments in two steps: firstly, a nucleotide fragment encoding a virus packaging protein was inserted into a host cell; and a nucleotide fragment that had a packaging signal sequence and contained a sequence of interest was then inserted into the packaging cell line constructed in the previous step to construct the virus vector producing cell line. The following example further demonstrates the effectiveness of the multi-transposon system in inserting multiple nucleotide fragments of interest into the genome of a host cell line, taking the construction of the stable virus vector producing cell line as an example. The method described in the present disclosure can significantly increase the virus production of the constructed stable lentivirus producing cell line. It will be appreciated by those skilled in the art that the method disclosed in the present disclosure are equally applicable to the construction of other complex cell lines involving the insertion of multiple nucleotide fragments of interest.

In this example, the expression cassettes of rev (SEQ ID NO: 97), VSV-G (SEQ ID NO: 98) and gag/pol (SEQ ID NO: 96) for lentivirus packaging, and a sequence (SEQ ID NO: 99) encoding activator rtTA and repressor CymR proteins for regulating their expressions were stably integrated into the genome of 293T cells to construct a lentivirus (LV) packaging cell line. A lentivirus genome transcription cassette (SEQ ID NO: 100, having the hPGK-luciferase-ires-EGFP sequence, only as an example of a nucleic acid fragment of interest, those skilled in the art would have been able to expect to construct any other nucleic acid sequence of interest using a similar method) carrying a nucleic acid fragment of interest was integrated into the genome of the LV packaging cell line constructed above by a second transposon system different from the first transposon system, thereby constructing the lentivirus producing cell line. The hygromycin resistance gene on SEQ ID NO: 99 was used to screen the LV packaging cell line, and the puromycin resistance gene on SEQ ID NO: 100 was used to screen the LV producing cell line. After antibiotic screening, the obtained LV producing cell line was cultured and induced with DOX (1 μg/ml, doxycycline hydrochloride, Sangon Biotech (Shanghai), A600889) and Cumate (200 μg/ml, Aladdin, 1107765) for lentivirus production. HT1080 cells were transduced with the lentiviruses produced after the induction of the above-mentioned LV producing cell lines constructed using various transposon system combinations, after which the transduction titers from the various LV producing cell lines were obtained by measuring luciferase activity. This example describes a method of constructing a LV producing cell line by firstly integrating rev, VSVG, gag and pol using a first transposon system, and then integrating a lentivirus genome transcription cassette carrying a nucleic acid fragment of interest through a second transposon system. Those skilled in the art would have been able to expect other combinations, that is, any one or more of rev, VSVG, gag, pol and a lentivirus genome transcription cassette carrying a nucleic acid fragment of interest can be integrated through a first transposon system, and then the remaining can be integrated through a second transposon system. Those skilled in the art also would have been able to expect the integration of the above-mentioned rev, VSVG, gag, pol and lentivirus genome transcription cassette carrying the nucleic acid fragment of interest using three, four, five or even more than five transposon systems.

The specific experimental process is briefly described below. 293T cells were seeded into a 60 mm culture dish at 1.5E+06 cells/dish and cultured in 3 ml of DMEM complete medium for 24 hours at 37° C. and 5% CO2. The cells were transfected according to the PEI method: during transfection, 500 μL of transfection reagent was added to each 60 mm culture dish, wherein the transfection reagent contained 5.6 μg of total plasmid. In the 5.6 μg of the plasmid, the transposon plasmid carrying gag/pol, the transposon plasmid carrying rev, the transposon plasmid carrying VSVG and the transposon plasmid carrying rtTA/CymR were 3.5 μg, 0.4 μg, 0.5 μg and 0.7 μg, respectively; and the plasmid carrying the first transposase gene was 0.5 μg. PEI MAX (Polysciences, 24765-1) was mixed with the plasmids at a mass ratio of 4:1, and after all mixtures were incubated for 15 minutes, the mixtures were added to the cells. After 24 hours, the medium was replaced with a DMEM complete medium supplemented with 200 μg/ml hygromycin. The cells were cultured under this condition for at least 3 consecutive passages until the cell line was stable. After that, the lentivirus genome transcription cassette containing the GOI was integrated into each packaging cell line using the second transposon system. The cells were again transfected according to the PEI method: during transfection, 500 μL of transfection reagent was added to each 60 mm culture dish, wherein the transfection reagent contained 4.3 μg of total plasmids comprising 4.0 μg of the transposon plasmid and 0.3 μg of the transposase plasmid. PEI MAX (Polysciences, 24765-1) was mixed with the plasmids at a mass ratio of 4:1, and after all mixtures were incubated for 15 minutes, the mixtures were added to the cells. After 24 hours, the medium was replaced with a DMEM complete medium supplemented with 2.5 μg/ml puromycin. The cells were cultured under this condition for at least 3 consecutive passages until the cell line was stable. Table 6 summarizes the plasmid combinations used in this example. The LV producing cell line constructed using a single transposon system was used as a control. A cell constructed using a single transposon plasmid but without transposase plasmid was used as a negative control. As mentioned above, 293T cells were seeded into a 60 mm culture dish at 1.5E6 cells/dish and cultured in 3 ml of DMEM complete medium for 24 hours at 37° C. and 5% CO2. The cells were transfected according to the PEI method: during transfection, 500 μl of transfection reagent was added to each 60 mm culture dish, wherein the transfection reagent contained 9.9 μg of plasmid. In the 9.9 μg of plasmid, the transposon plasmid carrying gag/pol, the transposon plasmid carrying rev, the transposon plasmid carrying VSVG, the transposon plasmid carrying rtTA/CymR, and the transposon plasmid carrying virus transcription cassette containing the GOI were 3.5 μg, 0.4 μg, 0.5 μg, 0.7 μg and 4.0 μg, respectively; and the plasmid carrying the transposase gene was 0.8 μg (the negative control used plasmid 06.01.1812 instead of the plasmid carrying the transposase gene). PEI MAX (Polysciences, 24765-1) was mixed with the plasmids at a mass ratio of 4:1, and after all mixtures were incubated for 15 minutes, the mixtures were added to the cells. After 24 hours, the medium was replaced with a DMEM complete medium supplemented with 2.5 μg/ml puromycin. The cells were cultured under this condition for at least 3 consecutive passages until the cell line was stable.

The virus production capacity of the stable lentivirus producing cell line was detected by luciferase assay after the transduction of HT1080 cells. Briefly, each cell line in Table 6 and Table 7 was seeded into a 6-well plate (Corning 3516) at 8E+05 cells/well and cultured in a DMEM complete medium at 37° C. and 5% CO2. After 24 hours of culture, the medium was replaced with a DMEM complete medium containing inducers, 1 μg/ml DOX (doxycycline hydrochloride, Sangon Biotech (Shanghai), A600889), 200 μg/ml Cumate (Aladdin, 1107765) and 5 mmol/L sodium butyrate (Sigma, 303410), to induce virus production. After 48 hours, the medium containing lentiviruses was collected and centrifuged at 14000 rpm for 10 minutes to collect the viral supernatant. At 24 hours before the virus sample was harvested, HT1080 cells were seeded in a 96-well plate (Corning 3916) at 1E+04 cells/well and cultured in a DMEM complete medium for 24 hours. At 1 hour before the virus sample was added to the HT1080 cells, the medium for the HT1080 cells was replaced with a DMEM complete medium containing 8 μg/ml polybrene (Sigam, H9268). After that, 50 μL of the virus sample was added to each well of the above-mentioned 96-well plate. After culture for additional 48 hours, the relative luciferase unit (RLU) of each well was detected using a Steady-Glo® luciferase assay system (Promega, E2610) kit according to the instructions for use (Promega, FB037), wherein the detection instrument was fluorescence microplate reader (Perkin Elmer VictorV). The results of the viral titers of the various lentivirus producing cell lines as measured by luciferase assay are summarized in Table 6 and Table 7.

It could be seen from Table 6 and Table 7, the viral titer (mean titer of 7.94+05TU (RLU)/mL) of the lentivirus producing cell line constructed using the dual-transposon system is significantly higher than the viral titer (mean titer of 1.94E+04TU (RLU)/mL) of the cell line constructed using the single transposon system. The mean titer of the viruses produced by the negative control cell line is 6.25E+02, which is close to the background value of luciferase assay. Furthermore, it could be seen from Table 6 and Table 7 below, the virus production capacity of the lentivirus producing cell line constructed using the combination of the Tol1, Tol2, ZB, Intruder, TcBuster, Yabusame-1, Uribo2, Sleeping Beauty and piggyBac transposon systems is significantly higher than that of the cell line constructed using other combinations, wherein the mean titer of the viruses produced by the lentivirus producing cell line constructed using the above transposon systems is 1.87E+06 TU (RLU)/mL.

TABLE 6
Construction of stable lentivirus producing cell line using dual-transposon system

| First transposon system | | | | | | Second transposon system | | | Titer |
|---|---|---|---|---|---|---|---|---|---|
| gag/pol | rev | VSVG | rtTA/CymR | Transposase | Antibiotic | GOI | Transposase | Antibiotic | TU/mL |
| 06.01.4301 | 06.01.4302 | 06.01.4303 | 06.01.4304 | 06.01.1494 | Hygromycin | 06.01.4362 | 06.01.1504 | Puromycin | 1.22E+06 |
| 06.01.4301 | 06.01.4302 | 06.01.4303 | 06.01.4304 | 06.01.1494 | Hygromycin | 06.01.4363 | 06.01.1527 | Puromycin | 6.59E+05 |
| 06.01.4301 | 06.01.4302 | 06.01.4303 | 06.01.4304 | 06.01.1494 | Hygromycin | 06.01.4364 | 06.01.1535 | Puromycin | 4.43E+05 |
| 06.01.4301 | 06.01.4302 | 06.01.4303 | 06.01.4304 | 06.01.1494 | Hygromycin | 06.01.4365 | 06.01.1538 | Puromycin | 2.80E+05 |
| 06.01.4301 | 06.01.4302 | 06.01.4303 | 06.01.4304 | 06.01.1494 | Hygromycin | 06.01.4366 | 06.01.1579 | Puromycin | 2.20E+05 |
| 06.01.4301 | 06.01.4302 | 06.01.4303 | 06.01.4304 | 06.01.1494 | Hygromycin | 06.01.4367 | 06.01.1573 | Puromycin | 7.83E+05 |
| 06.01.4301 | 06.01.4302 | 06.01.4303 | 06.01.4304 | 06.01.1494 | Hygromycin | 06.01.4368 | 06.01.1582 | Puromycin | 9.09E+05 |
| 06.01.4301 | 06.01.4302 | 06.01.4303 | 06.01.4304 | 06.01.1494 | Hygromycin | 06.01.4369 | 06.01.1596 | Puromycin | 4.50E+05 |
| 06.01.4301 | 06.01.4302 | 06.01.4303 | 06.01.4304 | 06.01.1494 | Hygromycin | 06.01.4370 | 06.01.1614 | Puromycin | 1.06E+06 |
| 06.01.4301 | 06.01.4302 | 06.01.4303 | 06.01.4304 | 06.01.1494 | Hygromycin | 06.01.4371 | 06.01.1679 | Puromycin | 5.21E+05 |
| 06.01.4301 | 06.01.4302 | 06.01.4303 | 06.01.4304 | 06.01.1494 | Hygromycin | 06.01.4372 | 06.01.1687 | Puromycin | 1.00E+06 |
| 06.01.4301 | 06.01.4302 | 06.01.4303 | 06.01.4304 | 06.01.1494 | Hygromycin | 06.01.4373 | 06.01.1770 | Puromycin | 1.30E+06 |
| 06.01.4301 | 06.01.4302 | 06.01.4303 | 06.01.4304 | 06.01.1494 | Hygromycin | 06.01.4374 | 06.01.1757 | Puromycin | 1.05E+06 |
| 06.01.4301 | 06.01.4302 | 06.01.4303 | 06.01.4304 | 06.01.1494 | Hygromycin | 06.01.4375 | 06.01.1807 | Puromycin | 1.48E+06 |
| 06.01.4305 | 06.01.4306 | 06.01.4307 | 06.01.4308 | 06.01.1504 | Hygromycin | 06.01.4361 | 06.01.1494 | Puromycin | 3.75E+05 |
| 06.01.4305 | 06.01.4306 | 06.01.4307 | 06.01.4308 | 06.01.1504 | Hygromycin | 06.01.4363 | 06.01.1527 | Puromycin | 1.30E+05 |
| 06.01.4305 | 06.01.4306 | 06.01.4307 | 06.01.4308 | 06.01.1504 | Hygromycin | 06.01.4364 | 06.01.1535 | Puromycin | 1.98E+05 |
| 06.01.4305 | 06.01.4306 | 06.01.4307 | 06.01.4308 | 06.01.1504 | Hygromycin | 06.01.4365 | 06.01.1538 | Puromycin | 8.87E+04 |
| 06.01.4305 | 06.01.4306 | 06.01.4307 | 06.01.4308 | 06.01.1504 | Hygromycin | 06.01.4366 | 06.01.1579 | Puromycin | 4.02E+04 |
| 06.01.4305 | 06.01.4306 | 06.01.4307 | 06.01.4308 | 06.01.1504 | Hygromycin | 06.01.4367 | 06.01.1573 | Puromycin | 2.56E+05 |
| 06.01.4305 | 06.01.4306 | 06.01.4307 | 06.01.4308 | 06.01.1504 | Hygromycin | 06.01.4368 | 06.01.1582 | Puromycin | 2.52E+05 |
| 06.01.4305 | 06.01.4306 | 06.01.4307 | 06.01.4308 | 06.01.1504 | Hygromycin | 06.01.4369 | 06.01.1596 | Puromycin | 1.10E+05 |
| 06.01.4305 | 06.01.4306 | 06.01.4307 | 06.01.4308 | 06.01.1504 | Hygromycin | 06.01.4370 | 06.01.1614 | Puromycin | 3.54E+05 |
| 06.01.4305 | 06.01.4306 | 06.01.4307 | 06.01.4308 | 06.01.1504 | Hygromycin | 06.01.4371 | 06.01.1679 | Puromycin | 9.10E+04 |
| 06.01.4305 | 06.01.4306 | 06.01.4307 | 06.01.4308 | 06.01.1504 | Hygromycin | 06.01.4372 | 06.01.1687 | Puromycin | 3.87E+05 |
| 06.01.4305 | 06.01.4306 | 06.01.4307 | 06.01.4308 | 06.01.1504 | Hygromycin | 06.01.4373 | 06.01.1770 | Puromycin | 3.18E+05 |
| 06.01.4305 | 06.01.4306 | 06.01.4307 | 06.01.4308 | 06.01.1504 | Hygromycin | 06.01.4374 | 06.01.1757 | Puromycin | 4.72E+05 |
| 06.01.4305 | 06.01.4306 | 06.01.4307 | 06.01.4308 | 06.01.1504 | Hygromycin | 06.01.4375 | 06.01.1807 | Puromycin | 5.66E+05 |
| 06.01.4309 | 06.01.4310 | 06.01.4311 | 06.01.4312 | 06.01.1527 | Hygromycin | 06.01.4361 | 06.01.1494 | Puromycin | 1.33E+04 |
| 06.01.4309 | 06.01.4310 | 06.01.4311 | 06.01.4312 | 06.01.1527 | Hygromycin | 06.01.4362 | 06.01.1504 | Puromycin | 1.00E+04 |
| 06.01.4309 | 06.01.4310 | 06.01.4311 | 06.01.4312 | 06.01.1527 | Hygromycin | 06.01.4364 | 06.01.1535 | Puromycin | 1.09E+04 |
| 06.01.4309 | 06.01.4310 | 06.01.4311 | 06.01.4312 | 06.01.1527 | Hygromycin | 06.01.4365 | 06.01.1538 | Puromycin | 6.93E+03 |
| 06.01.4309 | 06.01.4310 | 06.01.4311 | 06.01.4312 | 06.01.1527 | Hygromycin | 06.01.4366 | 06.01.1579 | Puromycin | 8.08E+03 |
| 06.01.4309 | 06.01.4310 | 06.01.4311 | 06.01.4312 | 06.01.1527 | Hygromycin | 06.01.4367 | 06.01.1573 | Puromycin | 7.44E+03 |
| 06.01.4309 | 06.01.4310 | 06.01.4311 | 06.01.4312 | 06.01.1527 | Hygromycin | 06.01.4368 | 06.01.1582 | Puromycin | 8.60E+03 |
| 06.01.4309 | 06.01.4310 | 06.01.4311 | 06.01.4312 | 06.01.1527 | Hygromycin | 06.01.4369 | 06.01.1596 | Puromycin | 8.74E+03 |
| 06.01.4309 | 06.01.4310 | 06.01.4311 | 06.01.4312 | 06.01.1527 | Hygromycin | 06.01.4370 | 06.01.1614 | Puromycin | 1.05E+04 |
| 06.01.4309 | 06.01.4310 | 06.01.4311 | 06.01.4312 | 06.01.1527 | Hygromycin | 06.01.4371 | 06.01.1679 | Puromycin | 1.13E+04 |
| 06.01.4309 | 06.01.4310 | 06.01.4311 | 06.01.4312 | 06.01.1527 | Hygromycin | 06.01.4372 | 06.01.1687 | Puromycin | 1.42E+04 |
| 06.01.4309 | 06.01.4310 | 06.01.4311 | 06.01.4312 | 06.01.1527 | Hygromycin | 06.01.4373 | 06.01.1770 | Puromycin | 1.06E+04 |
| 06.01.4309 | 06.01.4310 | 06.01.4311 | 06.01.4312 | 06.01.1527 | Hygromycin | 06.01.4374 | 06.01.1757 | Puromycin | 1.50E+04 |
| 06.01.4309 | 06.01.4310 | 06.01.4311 | 06.01.4312 | 06.01.1527 | Hygromycin | 06.01.4375 | 06.01.1807 | Puromycin | 1.47E+04 |
| 06.01.4313 | 06.01.4314 | 06.01.4315 | 06.01.4316 | 06.01.1535 | Hygromycin | 06.01.4361 | 06.01.1494 | Puromycin | 1.18E+04 |
| 06.01.4313 | 06.01.4314 | 06.01.4315 | 06.01.4316 | 06.01.1535 | Hygromycin | 06.01.4362 | 06.01.1504 | Puromycin | 7.60E+03 |
| 06.01.4313 | 06.01.4314 | 06.01.4315 | 06.01.4316 | 06.01.1535 | Hygromycin | 06.01.4363 | 06.01.1527 | Puromycin | 1.04E+04 |
| 06.01.4313 | 06.01.4314 | 06.01.4315 | 06.01.4316 | 06.01.1535 | Hygromycin | 06.01.4365 | 06.01.1538 | Puromycin | 1.31E+04 |
| 06.01.4313 | 06.01.4314 | 06.01.4315 | 06.01.4316 | 06.01.1535 | Hygromycin | 06.01.4366 | 06.01.1579 | Puromycin | 3.82E+03 |
| 06.01.4313 | 06.01.4314 | 06.01.4315 | 06.01.4316 | 06.01.1535 | Hygromycin | 06.01.4367 | 06.01.1573 | Puromycin | 1.28E+04 |
| 06.01.4313 | 06.01.4314 | 06.01.4315 | 06.01.4316 | 06.01.1535 | Hygromycin | 06.01.4368 | 06.01.1582 | Puromycin | 5.61E+03 |
| 06.01.4313 | 06.01.4314 | 06.01.4315 | 06.01.4316 | 06.01.1535 | Hygromycin | 06.01.4369 | 06.01.1596 | Puromycin | 6.30E+03 |
| 06.01.4313 | 06.01.4314 | 06.01.4315 | 06.01.4316 | 06.01.1535 | Hygromycin | 06.01.4370 | 06.01.1614 | Puromycin | 1.16E+04 |
| 06.01.4313 | 06.01.4314 | 06.01.4315 | 06.01.4316 | 06.01.1535 | Hygromycin | 06.01.4371 | 06.01.1679 | Puromycin | 5.74E+03 |
| 06.01.4313 | 06.01.4314 | 06.01.4315 | 06.01.4316 | 06.01.1535 | Hygromycin | 06.01.4372 | 06.01.1687 | Puromycin | 7.07E+03 |
| 06.01.4313 | 06.01.4314 | 06.01.4315 | 06.01.4316 | 06.01.1535 | Hygromycin | 06.01.4373 | 06.01.1770 | Puromycin | 1.03E+04 |
| 06.01.4313 | 06.01.4314 | 06.01.4315 | 06.01.4316 | 06.01.1535 | Hygromycin | 06.01.4374 | 06.01.1757 | Puromycin | 9.99E+03 |
| 06.01.4313 | 06.01.4314 | 06.01.4315 | 06.01.4316 | 06.01.1535 | Hygromycin | 06.01.4375 | 06.01.1807 | Puromycin | 1.08E+04 |
| 06.01.4317 | 06.01.4318 | 06.01.4319 | 06.01.4320 | 06.01.1538 | Hygromycin | 06.01.4361 | 06.01.1494 | Puromycin | 8.72E+03 |
| 06.01.4317 | 06.01.4318 | 06.01.4319 | 06.01.4320 | 06.01.1538 | Hygromycin | 06.01.4362 | 06.01.1504 | Puromycin | 7.14E+03 |
| 06.01.4317 | 06.01.4318 | 06.01.4319 | 06.01.4320 | 06.01.1538 | Hygromycin | 06.01.4363 | 06.01.1527 | Puromycin | 5.80E+03 |
| 06.01.4317 | 06.01.4318 | 06.01.4319 | 06.01.4320 | 06.01.1538 | Hygromycin | 06.01.4364 | 06.01.1535 | Puromycin | 5.69E+03 |
| 06.01.4317 | 06.01.4318 | 06.01.4319 | 06.01.4320 | 06.01.1538 | Hygromycin | 06.01.4366 | 06.01.1579 | Puromycin | 4.92E+03 |
| 06.01.4317 | 06.01.4318 | 06.01.4319 | 06.01.4320 | 06.01.1538 | Hygromycin | 06.01.4367 | 06.01.1573 | Puromycin | 3.77E+03 |
| 06.01.4317 | 06.01.4318 | 06.01.4319 | 06.01.4320 | 06.01.1538 | Hygromycin | 06.01.4368 | 06.01.1582 | Puromycin | 3.43E+03 |
| 06.01.4317 | 06.01.4318 | 06.01.4319 | 06.01.4320 | 06.01.1538 | Hygromycin | 06.01.4369 | 06.01.1596 | Puromycin | 4.69E+03 |
| 06.01.4317 | 06.01.4318 | 06.01.4319 | 06.01.4320 | 06.01.1538 | Hygromycin | 06.01.4370 | 06.01.1614 | Puromycin | 9.03E+03 |
| 06.01.4317 | 06.01.4318 | 06.01.4319 | 06.01.4320 | 06.01.1538 | Hygromycin | 06.01.4371 | 06.01.1679 | Puromycin | 4.12E+03 |
| 06.01.4317 | 06.01.4318 | 06.01.4319 | 06.01.4320 | 06.01.1538 | Hygromycin | 06.01.4372 | 06.01.1687 | Puromycin | 6.13E+03 |
| 06.01.4317 | 06.01.4318 | 06.01.4319 | 06.01.4320 | 06.01.1538 | Hygromycin | 06.01.4373 | 06.01.1770 | Puromycin | 6.72E+03 |
| 06.01.4317 | 06.01.4318 | 06.01.4319 | 06.01.4320 | 06.01.1538 | Hygromycin | 06.01.4374 | 06.01.1757 | Puromycin | 9.24E+03 |
| 06.01.4317 | 06.01.4318 | 06.01.4319 | 06.01.4320 | 06.01.1538 | Hygromycin | 06.01.4375 | 06.01.1807 | Puromycin | 9.03E+03 |
| 06.01.4321 | 06.01.4322 | 06.01.4323 | 06.01.4324 | 06.01.1579 | Hygromycin | 06.01.4361 | 06.01.1494 | Puromycin | 1.99E+03 |
| 06.01.4321 | 06.01.4322 | 06.01.4323 | 06.01.4324 | 06.01.1579 | Hygromycin | 06.01.4362 | 06.01.1504 | Puromycin | 2.62E+03 |
| 06.01.4321 | 06.01.4322 | 06.01.4323 | 06.01.4324 | 06.01.1579 | Hygromycin | 06.01.4363 | 06.01.1527 | Puromycin | 3.03E+03 |
| 06.01.4321 | 06.01.4322 | 06.01.4323 | 06.01.4324 | 06.01.1579 | Hygromycin | 06.01.4364 | 06.01.1535 | Puromycin | 2.22E+03 |
| 06.01.4321 | 06.01.4322 | 06.01.4323 | 06.01.4324 | 06.01.1579 | Hygromycin | 06.01.4365 | 06.01.1538 | Puromycin | 2.13E+03 |
| 06.01.4321 | 06.01.4322 | 06.01.4323 | 06.01.4324 | 06.01.1579 | Hygromycin | 06.01.4367 | 06.01.1573 | Puromycin | 3.42E+03 |
| 06.01.4321 | 06.01.4322 | 06.01.4323 | 06.01.4324 | 06.01.1579 | Hygromycin | 06.01.4368 | 06.01.1582 | Puromycin | 4.40E+03 |
| 06.01.4321 | 06.01.4322 | 06.01.4323 | 06.01.4324 | 06.01.1579 | Hygromycin | 06.01.4369 | 06.01.1596 | Puromycin | 1.80E+03 |
| 06.01.4321 | 06.01.4322 | 06.01.4323 | 06.01.4324 | 06.01.1579 | Hygromycin | 06.01.4370 | 06.01.1614 | Puromycin | 3.19E+03 |
| 06.01.4321 | 06.01.4322 | 06.01.4323 | 06.01.4324 | 06.01.1579 | Hygromycin | 06.01.4371 | 06.01.1679 | Puromycin | 2.75E+03 |
| 06.01.4321 | 06.01.4322 | 06.01.4323 | 06.01.4324 | 06.01.1579 | Hygromycin | 06.01.4372 | 06.01.1687 | Puromycin | 1.70E+03 |
| 06.01.4321 | 06.01.4322 | 06.01.4323 | 06.01.4324 | 06.01.1579 | Hygromycin | 06.01.4373 | 06.01.1770 | Puromycin | 2.92E+03 |
| 06.01.4321 | 06.01.4322 | 06.01.4323 | 06.01.4324 | 06.01.1579 | Hygromycin | 06.01.4374 | 06.01.1757 | Puromycin | 2.98E+03 |
| 06.01.4321 | 06.01.4322 | 06.01.4323 | 06.01.4324 | 06.01.1579 | Hygromycin | 06.01.4375 | 06.01.1807 | Puromycin | 1.57E+03 |
| 06.01.4325 | 06.01.4326 | 06.01.4327 | 06.01.4328 | 06.01.1573 | Hygromycin | 06.01.4361 | 06.01.1494 | Puromycin | 1.28E+06 |
| 06.01.4325 | 06.01.4326 | 06.01.4327 | 06.01.4328 | 06.01.1573 | Hygromycin | 06.01.4362 | 06.01.1504 | Puromycin | 8.97E+05 |
| 06.01.4325 | 06.01.4326 | 06.01.4327 | 06.01.4328 | 06.01.1573 | Hygromycin | 06.01.4363 | 06.01.1527 | Puromycin | 3.39E+05 |
| 06.01.4325 | 06.01.4326 | 06.01.4327 | 06.01.4328 | 06.01.1573 | Hygromycin | 06.01.4364 | 06.01.1535 | Puromycin | 7.05E+05 |
| 06.01.4325 | 06.01.4326 | 06.01.4327 | 06.01.4328 | 06.01.1573 | Hygromycin | 06.01.4365 | 06.01.1538 | Puromycin | 3.13E+05 |
| 06.01.4325 | 06.01.4326 | 06.01.4327 | 06.01.4328 | 06.01.1573 | Hygromycin | 06.01.4366 | 06.01.1579 | Puromycin | 2.51E+05 |
| 06.01.4325 | 06.01.4326 | 06.01.4327 | 06.01.4328 | 06.01.1573 | Hygromycin | 06.01.4368 | 06.01.1582 | Puromycin | 7.55E+05 |
| 06.01.4325 | 06.01.4326 | 06.01.4327 | 06.01.4328 | 06.01.1573 | Hygromycin | 06.01.4369 | 06.01.1596 | Puromycin | 5.83E+05 |
| 06.01.4325 | 06.01.4326 | 06.01.4327 | 06.01.4328 | 06.01.1573 | Hygromycin | 06.01.4370 | 06.01.1614 | Puromycin | 1.38E+06 |
| 06.01.4325 | 06.01.4326 | 06.01.4327 | 06.01.4328 | 06.01.1573 | Hygromycin | 06.01.4371 | 06.01.1679 | Puromycin | 4.19E+05 |
| 06.01.4325 | 06.01.4326 | 06.01.4327 | 06.01.4328 | 06.01.1573 | Hygromycin | 06.01.4372 | 06.01.1687 | Puromycin | 9.29E+05 |
| 06.01.4325 | 06.01.4326 | 06.01.4327 | 06.01.4328 | 06.01.1573 | Hygromycin | 06.01.4373 | 06.01.1770 | Puromycin | 1.61E+06 |
| 06.01.4325 | 06.01.4326 | 06.01.4327 | 06.01.4328 | 06.01.1573 | Hygromycin | 06.01.4374 | 06.01.1757 | Puromycin | 8.96E+05 |
| 06.01.4325 | 06.01.4326 | 06.01.4327 | 06.01.4328 | 06.01.1573 | Hygromycin | 06.01.4375 | 06.01.1807 | Puromycin | 1.50E+06 |
| 06.01.4329 | 06.01.4330 | 06.01.4331 | 06.01.4332 | 06.01.1582 | Hygromycin | 06.01.4361 | 06.01.1494 | Puromycin | 1.30E+06 |
| 06.01.4329 | 06.01.4330 | 06.01.4331 | 06.01.4332 | 06.01.1582 | Hygromycin | 06.01.4362 | 06.01.1504 | Puromycin | 8.76E+05 |
| 06.01.4329 | 06.01.4330 | 06.01.4331 | 06.01.4332 | 06.01.1582 | Hygromycin | 06.01.4363 | 06.01.1527 | Puromycin | 1.06E+05 |
| 06.01.4329 | 06.01.4330 | 06.01.4331 | 06.01.4332 | 06.01.1582 | Hygromycin | 06.01.4364 | 06.01.1535 | Puromycin | 2.84E+05 |
| 06.01.4329 | 06.01.4330 | 06.01.4331 | 06.01.4332 | 06.01.1582 | Hygromycin | 06.01.4365 | 06.01.1538 | Puromycin | 3.23E+05 |
| 06.01.4329 | 06.01.4330 | 06.01.4331 | 06.01.4332 | 06.01.1582 | Hygromycin | 06.01.4366 | 06.01.1579 | Puromycin | 2.29E+05 |
| 06.01.4329 | 06.01.4330 | 06.01.4331 | 06.01.4332 | 06.01.1582 | Hygromycin | 06.01.4367 | 06.01.1573 | Puromycin | 9.42E+05 |
| 06.01.4329 | 06.01.4330 | 06.01.4331 | 06.01.4332 | 06.01.1582 | Hygromycin | 06.01.4369 | 06.01.1596 | Puromycin | 5.58E+05 |
| 06.01.4329 | 06.01.4330 | 06.01.4331 | 06.01.4332 | 06.01.1582 | Hygromycin | 06.01.4370 | 06.01.1614 | Puromycin | 1.02E+06 |
| 06.01.4329 | 06.01.4330 | 06.01.4331 | 06.01.4332 | 06.01.1582 | Hygromycin | 06.01.4371 | 06.01.1679 | Puromycin | 4.60E+05 |
| 06.01.4329 | 06.01.4330 | 06.01.4331 | 06.01.4332 | 06.01.1582 | Hygromycin | 06.01.4372 | 06.01.1687 | Puromycin | 8.14E+05 |
| 06.01.4329 | 06.01.4330 | 06.01.4331 | 06.01.4332 | 06.01.1582 | Hygromycin | 06.01.4373 | 06.01.1770 | Puromycin | 1.09E+06 |
| 06.01.4329 | 06.01.4330 | 06.01.4331 | 06.01.4332 | 06.01.1582 | Hygromycin | 06.01.4374 | 06.01.1757 | Puromycin | 1.16E+06 |
| 06.01.4329 | 06.01.4330 | 06.01.4331 | 06.01.4332 | 06.01.1582 | Hygromycin | 06.01.4375 | 06.01.1807 | Puromycin | 1.14E+06 |
| 06.01.4333 | 06.01.4334 | 06.01.4335 | 06.01.4336 | 06.01.1596 | Hygromycin | 06.01.4361 | 06.01.1494 | Puromycin | 9.07E+04 |
| 06.01.4333 | 06.01.4334 | 06.01.4335 | 06.01.4336 | 06.01.1596 | Hygromycin | 06.01.4362 | 06.01.1504 | Puromycin | 1.63E+05 |
| 06.01.4333 | 06.01.4334 | 06.01.4335 | 06.01.4336 | 06.01.1596 | Hygromycin | 06.01.4363 | 06.01.1527 | Puromycin | 1.06E+05 |
| 06.01.4333 | 06.01.4334 | 06.01.4335 | 06.01.4336 | 06.01.1596 | Hygromycin | 06.01.4364 | 06.01.1535 | Puromycin | 8.50E+04 |
| 06.01.4333 | 06.01.4334 | 06.01.4335 | 06.01.4336 | 06.01.1596 | Hygromycin | 06.01.4365 | 06.01.1538 | Puromycin | 9.17E+04 |
| 06.01.4333 | 06.01.4334 | 06.01.4335 | 06.01.4336 | 06.01.1596 | Hygromycin | 06.01.4366 | 06.01.1579 | Puromycin | 6.45E+04 |
| 06.01.4333 | 06.01.4334 | 06.01.4335 | 06.01.4336 | 06.01.1596 | Hygromycin | 06.01.4367 | 06.01.1573 | Puromycin | 1.11E+05 |
| 06.01.4333 | 06.01.4334 | 06.01.4335 | 06.01.4336 | 06.01.1596 | Hygromycin | 06.01.4368 | 06.01.1582 | Puromycin | 6.45E+04 |
| 06.01.4333 | 06.01.4334 | 06.01.4335 | 06.01.4336 | 06.01.1596 | Hygromycin | 06.01.4370 | 06.01.1614 | Puromycin | 1.57E+05 |
| 06.01.4333 | 06.01.4334 | 06.01.4335 | 06.01.4336 | 06.01.1596 | Hygromycin | 06.01.4371 | 06.01.1679 | Puromycin | 9.10E+04 |
| 06.01.4333 | 06.01.4334 | 06.01.4335 | 06.01.4336 | 06.01.1596 | Hygromycin | 06.01.4372 | 06.01.1687 | Puromycin | 1.35E+05 |
| 06.01.4333 | 06.01.4334 | 06.01.4335 | 06.01.4336 | 06.01.1596 | Hygromycin | 06.01.4373 | 06.01.1770 | Puromycin | 1.14E+05 |
| 06.01.4333 | 06.01.4334 | 06.01.4335 | 06.01.4336 | 06.01.1596 | Hygromycin | 06.01.4374 | 06.01.1757 | Puromycin | 1.75E+05 |
| 06.01.4333 | 06.01.4334 | 06.01.4335 | 06.01.4336 | 06.01.1596 | Hygromycin | 06.01.4375 | 06.01.1807 | Puromycin | 1.11E+05 |
| 06.01.4337 | 06.01.4338 | 06.01.4339 | 06.01.4340 | 06.01.1614 | Hygromycin | 06.01.4361 | 06.01.1494 | Puromycin | 1.45E+06 |
| 06.01.4337 | 06.01.4338 | 06.01.4339 | 06.01.4340 | 06.01.1614 | Hygromycin | 06.01.4362 | 06.01.1504 | Puromycin | 1.20E+06 |
| 06.01.4337 | 06.01.4338 | 06.01.4339 | 06.01.4340 | 06.01.1614 | Hygromycin | 06.01.4363 | 06.01.1527 | Puromycin | 6.71E+05 |
| 06.01.4337 | 06.01.4338 | 06.01.4339 | 06.01.4340 | 06.01.1614 | Hygromycin | 06.01.4364 | 06.01.1535 | Puromycin | 8.21E+05 |
| 06.01.4337 | 06.01.4338 | 06.01.4339 | 06.01.4340 | 06.01.1614 | Hygromycin | 06.01.4365 | 06.01.1538 | Puromycin | 1.82E+05 |
| 06.01.4337 | 06.01.4338 | 06.01.4339 | 06.01.4340 | 06.01.1614 | Hygromycin | 06.01.4366 | 06.01.1579 | Puromycin | 2.68E+05 |
| 06.01.4337 | 06.01.4338 | 06.01.4339 | 06.01.4340 | 06.01.1614 | Hygromycin | 06.01.4367 | 06.01.1573 | Puromycin | 7.61E+05 |
| 06.01.4337 | 06.01.4338 | 06.01.4339 | 06.01.4340 | 06.01.1614 | Hygromycin | 06.01.4368 | 06.01.1582 | Puromycin | 8.58E+05 |
| 06.01.4337 | 06.01.4338 | 06.01.4339 | 06.01.4340 | 06.01.1614 | Hygromycin | 06.01.4369 | 06.01.1596 | Puromycin | 5.34E+05 |
| 06.01.4337 | 06.01.4338 | 06.01.4339 | 06.01.4340 | 06.01.1614 | Hygromycin | 06.01.4371 | 06.01.1679 | Puromycin | 9.06E+05 |
| 06.01.4337 | 06.01.4338 | 06.01.4339 | 06.01.4340 | 06.01.1614 | Hygromycin | 06.01.4372 | 06.01.1687 | Puromycin | 2.05E+06 |
| 06.01.4337 | 06.01.4338 | 06.01.4339 | 06.01.4340 | 06.01.1614 | Hygromycin | 06.01.4373 | 06.01.1770 | Puromycin | 1.60E+06 |
| 06.01.4337 | 06.01.4338 | 06.01.4339 | 06.01.4340 | 06.01.1614 | Hygromycin | 06.01.4374 | 06.01.1757 | Puromycin | 1.53E+06 |
| 06.01.4337 | 06.01.4338 | 06.01.4339 | 06.01.4340 | 06.01.1614 | Hygromycin | 06.01.4375 | 06.01.1807 | Puromycin | 1.89E+06 |
| 06.01.4341 | 06.01.4342 | 06.01.4343 | 06.01.4344 | 06.01.1679 | Hygromycin | 06.01.4361 | 06.01.1494 | Puromycin | 2.79E+04 |
| 06.01.4341 | 06.01.4342 | 06.01.4343 | 06.01.4344 | 06.01.1679 | Hygromycin | 06.01.4362 | 06.01.1504 | Puromycin | 3.70E+04 |
| 06.01.4341 | 06.01.4342 | 06.01.4343 | 06.01.4344 | 06.01.1679 | Hygromycin | 06.01.4363 | 06.01.1527 | Puromycin | 1.93E+04 |
| 06.01.4341 | 06.01.4342 | 06.01.4343 | 06.01.4344 | 06.01.1679 | Hygromycin | 06.01.4364 | 06.01.1535 | Puromycin | 1.31E+04 |
| 06.01.4341 | 06.01.4342 | 06.01.4343 | 06.01.4344 | 06.01.1679 | Hygromycin | 06.01.4365 | 06.01.1538 | Puromycin | 1.66E+04 |
| 06.01.4341 | 06.01.4342 | 06.01.4343 | 06.01.4344 | 06.01.1679 | Hygromycin | 06.01.4366 | 06.01.1579 | Puromycin | 7.75E+03 |
| 06.01.4341 | 06.01.4342 | 06.01.4343 | 06.01.4344 | 06.01.1679 | Hygromycin | 06.01.4367 | 06.01.1573 | Puromycin | 2.62E+04 |
| 06.01.4341 | 06.01.4342 | 06.01.4343 | 06.01.4344 | 06.01.1679 | Hygromycin | 06.01.4368 | 06.01.1582 | Puromycin | 2.58E+04 |
| 06.01.4341 | 06.01.4342 | 06.01.4343 | 06.01.4344 | 06.01.1679 | Hygromycin | 06.01.4369 | 06.01.1596 | Puromycin | 2.89E+04 |
| 06.01.4341 | 06.01.4342 | 06.01.4343 | 06.01.4344 | 06.01.1679 | Hygromycin | 06.01.4370 | 06.01.1614 | Puromycin | 2.48E+04 |
| 06.01.4341 | 06.01.4342 | 06.01.4343 | 06.01.4344 | 06.01.1679 | Hygromycin | 06.01.4372 | 06.01.1687 | Puromycin | 3.34E+04 |
| 06.01.4341 | 06.01.4342 | 06.01.4343 | 06.01.4344 | 06.01.1679 | Hygromycin | 06.01.4373 | 06.01.1770 | Puromycin | 1.68E+04 |
| 06.01.4341 | 06.01.4342 | 06.01.4343 | 06.01.4344 | 06.01.1679 | Hygromycin | 06.01.4374 | 06.01.1757 | Puromycin | 4.54E+04 |
| 06.01.4341 | 06.01.4342 | 06.01.4343 | 06.01.4344 | 06.01.1679 | Hygromycin | 06.01.4375 | 06.01.1807 | Puromycin | 3.90E+04 |
| 06.01.4345 | 06.01.4346 | 06.01.4347 | 06.01.4348 | 06.01.1687 | Hygromycin | 06.01.4361 | 06.01.1494 | Puromycin | 2.14E+06 |
| 06.01.4345 | 06.01.4346 | 06.01.4347 | 06.01.4348 | 06.01.1687 | Hygromycin | 06.01.4362 | 06.01.1504 | Puromycin | 2.70E+06 |
| 06.01.4345 | 06.01.4346 | 06.01.4347 | 06.01.4348 | 06.01.1687 | Hygromycin | 06.01.4363 | 06.01.1527 | Puromycin | 1.60E+06 |
| 06.01.4345 | 06.01.4346 | 06.01.4347 | 06.01.4348 | 06.01.1687 | Hygromycin | 06.01.4364 | 06.01.1535 | Puromycin | 1.49E+06 |
| 06.01.4345 | 06.01.4346 | 06.01.4347 | 06.01.4348 | 06.01.1687 | Hygromycin | 06.01.4365 | 06.01.1538 | Puromycin | 4.99E+05 |
| 06.01.4345 | 06.01.4346 | 06.01.4347 | 06.01.4348 | 06.01.1687 | Hygromycin | 06.01.4366 | 06.01.1579 | Puromycin | 5.28E+05 |
| 06.01.4345 | 06.01.4346 | 06.01.4347 | 06.01.4348 | 06.01.1687 | Hygromycin | 06.01.4367 | 06.01.1573 | Puromycin | 1.60E+06 |
| 06.01.4345 | 06.01.4346 | 06.01.4347 | 06.01.4348 | 06.01.1687 | Hygromycin | 06.01.4368 | 06.01.1582 | Puromycin | 1.12E+06 |
| 06.01.4345 | 06.01.4346 | 06.01.4347 | 06.01.4348 | 06.01.1687 | Hygromycin | 06.01.4369 | 06.01.1596 | Puromycin | 1.37E+06 |
| 06.01.4345 | 06.01.4346 | 06.01.4347 | 06.01.4348 | 06.01.1687 | Hygromycin | 06.01.4370 | 06.01.1614 | Puromycin | 2.70E+06 |
| 06.01.4345 | 06.01.4346 | 06.01.4347 | 06.01.4348 | 06.01.1687 | Hygromycin | 06.01.4371 | 06.01.1679 | Puromycin | 5.68E+05 |
| 06.01.4345 | 06.01.4346 | 06.01.4347 | 06.01.4348 | 06.01.1687 | Hygromycin | 06.01.4373 | 06.01.1770 | Puromycin | 3.13E+06 |
| 06.01.4345 | 06.01.4346 | 06.01.4347 | 06.01.4348 | 06.01.1687 | Hygromycin | 06.01.4374 | 06.01.1757 | Puromycin | 1.83E+06 |
| 06.01.4345 | 06.01.4346 | 06.01.4347 | 06.01.4348 | 06.01.1687 | Hygromycin | 06.01.4375 | 06.01.1807 | Puromycin | 2.82E+06 |
| 06.01.4349 | 06.01.4350 | 06.01.4351 | 06.01.4352 | 06.01.1770 | Hygromycin | 06.01.4361 | 06.01.1494 | Puromycin | 1.87E+06 |
| 06.01.4349 | 06.01.4350 | 06.01.4351 | 06.01.4352 | 06.01.1770 | Hygromycin | 06.01.4362 | 06.01.1504 | Puromycin | 1.47E+06 |
| 06.01.4349 | 06.01.4350 | 06.01.4351 | 06.01.4352 | 06.01.1770 | Hygromycin | 06.01.4363 | 06.01.1527 | Puromycin | 6.50E+05 |
| 06.01.4349 | 06.01.4350 | 06.01.4351 | 06.01.4352 | 06.01.1770 | Hygromycin | 06.01.4364 | 06.01.1535 | Puromycin | 1.16E+06 |
| 06.01.4349 | 06.01.4350 | 06.01.4351 | 06.01.4352 | 06.01.1770 | Hygromycin | 06.01.4365 | 06.01.1538 | Puromycin | 1.64E+06 |
| 06.01.4349 | 06.01.4350 | 06.01.4351 | 06.01.4352 | 06.01.1770 | Hygromycin | 06.01.4366 | 06.01.1579 | Puromycin | 1.70E+05 |
| 06.01.4349 | 06.01.4350 | 06.01.4351 | 06.01.4352 | 06.01.1770 | Hygromycin | 06.01.4367 | 06.01.1573 | Puromycin | 2.31E+06 |
| 06.01.4349 | 06.01.4350 | 06.01.4351 | 06.01.4352 | 06.01.1770 | Hygromycin | 06.01.4368 | 06.01.1582 | Puromycin | 1.69E+06 |
| 06.01.4349 | 06.01.4350 | 06.01.4351 | 06.01.4352 | 06.01.1770 | Hygromycin | 06.01.4369 | 06.01.1596 | Puromycin | 6.77E+05 |
| 06.01.4349 | 06.01.4350 | 06.01.4351 | 06.01.4352 | 06.01.1770 | Hygromycin | 06.01.4370 | 06.01.1614 | Puromycin | 2.54E+06 |
| 06.01.4349 | 06.01.4350 | 06.01.4351 | 06.01.4352 | 06.01.1770 | Hygromycin | 06.01.4371 | 06.01.1679 | Puromycin | 1.04E+06 |
| 06.01.4349 | 06.01.4350 | 06.01.4351 | 06.01.4352 | 06.01.1770 | Hygromycin | 06.01.4372 | 06.01.1687 | Puromycin | 1.91E+06 |
| 06.01.4349 | 06.01.4350 | 06.01.4351 | 06.01.4352 | 06.01.1770 | Hygromycin | 06.01.4374 | 06.01.1757 | Puromycin | 3.48E+06 |
| 06.01.4349 | 06.01.4350 | 06.01.4351 | 06.01.4352 | 06.01.1770 | Hygromycin | 06.01.4375 | 06.01.1807 | Puromycin | 3.90E+06 |
| 06.01.4353 | 06.01.4354 | 06.01.4355 | 06.01.4356 | 06.01.1757 | Hygromycin | 06.01.4361 | 06.01.1494 | Puromycin | 4.55E+06 |
| 06.01.4353 | 06.01.4354 | 06.01.4355 | 06.01.4356 | 06.01.1757 | Hygromycin | 06.01.4362 | 06.01.1504 | Puromycin | 3.89E+06 |
| 06.01.4353 | 06.01.4354 | 06.01.4355 | 06.01.4356 | 06.01.1757 | Hygromycin | 06.01.4363 | 06.01.1527 | Puromycin | 1.05E+06 |
| 06.01.4353 | 06.01.4354 | 06.01.4355 | 06.01.4356 | 06.01.1757 | Hygromycin | 06.01.4364 | 06.01.1535 | Puromycin | 9.85E+05 |
| 06.01.4353 | 06.01.4354 | 06.01.4355 | 06.01.4356 | 06.01.1757 | Hygromycin | 06.01.4365 | 06.01.1538 | Puromycin | 2.27E+05 |
| 06.01.4353 | 06.01.4354 | 06.01.4355 | 06.01.4356 | 06.01.1757 | Hygromycin | 06.01.4366 | 06.01.1579 | Puromycin | 1.01E+05 |
| 06.01.4353 | 06.01.4354 | 06.01.4355 | 06.01.4356 | 06.01.1757 | Hygromycin | 06.01.4367 | 06.01.1573 | Puromycin | 2.87E+06 |
| 06.01.4353 | 06.01.4354 | 06.01.4355 | 06.01.4356 | 06.01.1757 | Hygromycin | 06.01.4368 | 06.01.1582 | Puromycin | 2.55E+06 |
| 06.01.4353 | 06.01.4354 | 06.01.4355 | 06.01.4356 | 06.01.1757 | Hygromycin | 06.01.4369 | 06.01.1596 | Puromycin | 9.15E+05 |
| 06.01.4353 | 06.01.4354 | 06.01.4355 | 06.01.4356 | 06.01.1757 | Hygromycin | 06.01.4370 | 06.01.1614 | Puromycin | 3.81E+06 |
| 06.01.4353 | 06.01.4354 | 06.01.4355 | 06.01.4356 | 06.01.1757 | Hygromycin | 06.01.4371 | 06.01.1679 | Puromycin | 6.93E+05 |
| 06.01.4353 | 06.01.4354 | 06.01.4355 | 06.01.4356 | 06.01.1757 | Hygromycin | 06.01.4372 | 06.01.1687 | Puromycin | 4.00E+06 |
| 06.01.4353 | 06.01.4354 | 06.01.4355 | 06.01.4356 | 06.01.1757 | Hygromycin | 06.01.4373 | 06.01.1770 | Puromycin | 5.78E+06 |
| 06.01.4353 | 06.01.4354 | 06.01.4355 | 06.01.4356 | 06.01.1757 | Hygromycin | 06.01.4375 | 06.01.1807 | Puromycin | 4.15E+06 |
| 06.01.4357 | 06.01.4358 | 06.01.4359 | 06.01.4360 | 06.01.1807 | Hygromycin | 06.01.4361 | 06.01.1494 | Puromycin | 2.71E+06 |
| 06.01.4357 | 06.01.4358 | 06.01.4359 | 06.01.4360 | 06.01.1807 | Hygromycin | 06.01.4362 | 06.01.1504 | Puromycin | 2.05E+06 |
| 06.01.4357 | 06.01.4358 | 06.01.4359 | 06.01.4360 | 06.01.1807 | Hygromycin | 06.01.4363 | 06.01.1527 | Puromycin | 3.67E+05 |
| 06.01.4357 | 06.01.4358 | 06.01.4359 | 06.01.4360 | 06.01.1807 | Hygromycin | 06.01.4364 | 06.01.1535 | Puromycin | 4.60E+05 |
| 06.01.4357 | 06.01.4358 | 06.01.4359 | 06.01.4360 | 06.01.1807 | Hygromycin | 06.01.4365 | 06.01.1538 | Puromycin | 5.50E+05 |
| 06.01.4357 | 06.01.4358 | 06.01.4359 | 06.01.4360 | 06.01.1807 | Hygromycin | 06.01.4366 | 06.01.1579 | Puromycin | 6.23E+04 |
| 06.01.4357 | 06.01.4358 | 06.01.4359 | 06.01.4360 | 06.01.1807 | Hygromycin | 06.01.4367 | 06.01.1573 | Puromycin | 1.88E+06 |
| 06.01.4357 | 06.01.4358 | 06.01.4359 | 06.01.4360 | 06.01.1807 | Hygromycin | 06.01.4368 | 06.01.1582 | Puromycin | 1.32E+06 |
| 06.01.4357 | 06.01.4358 | 06.01.4359 | 06.01.4360 | 06.01.1807 | Hygromycin | 06.01.4369 | 06.01.1596 | Puromycin | 1.69E+05 |
| 06.01.4357 | 06.01.4358 | 06.01.4359 | 06.01.4360 | 06.01.1807 | Hygromycin | 06.01.4370 | 06.01.1614 | Puromycin | 3.83E+06 |
| 06.01.4357 | 06.01.4358 | 06.01.4359 | 06.01.4360 | 06.01.1807 | Hygromycin | 06.01.4371 | 06.01.1679 | Puromycin | 1.63E+06 |
| 06.01.4357 | 06.01.4358 | 06.01.4359 | 06.01.4360 | 06.01.1807 | Hygromycin | 06.01.4372 | 06.01.1687 | Puromycin | 3.70E+06 |
| 06.01.4357 | 06.01.4358 | 06.01.4359 | 06.01.4360 | 06.01.1807 | Hygromycin | 06.01.4373 | 06.01.1770 | Puromycin | 4.40E+06 |
| 06.01.4357 | 06.01.4358 | 06.01.4359 | 06.01.4360 | 06.01.1807 | Hygromycin | 06.01.4374 | 06.01.1757 | Puromycin | 5.22E+06 |

TABLE 7
Construction of stable lentivirus producing cell line using single transposon system

| First transposon system | | | | | | | Titer |
|---|---|---|---|---|---|---|---|
| gag/pol | rev | VSVG | rtTA/CymR | GOI | Transposase | Antibiotic | TU/mL |
| 06.01.4301 | 06.01.4302 | 06.01.4303 | 06.01.4304 | 06.01.4361 | 06.01.1494 | Hygromycin + puromycin | 2.90E+04 |
| 06.01.4305 | 06.01.4306 | 06.01.4307 | 06.01.4308 | 06.01.4362 | 06.01.1504 | Hygromycin + puromycin | 1.77E+04 |
| 06.01.4309 | 06.01.4310 | 06.01.4311 | 06.01.4312 | 06.01.4363 | 06.01.1527 | Hygromycin + puromycin | 1.38E+03 |
| 06.01.4313 | 06.01.4314 | 06.01.4315 | 06.01.4316 | 06.01.4364 | 06.01.1535 | Hygromycin + puromycin | 1.93E+03 |
| 06.01.4317 | 06.01.4318 | 06.01.4319 | 06.01.4320 | 06.01.4365 | 06.01.1538 | Hygromycin + puromycin | 1.89E+03 |
| 06.01.4321 | 06.01.4322 | 06.01.4323 | 06.01.4324 | 06.01.4366 | 06.01.1579 | Hygromycin + puromycin | 1.34E+03 |
| 06.01.4325 | 06.01.4326 | 06.01.4327 | 06.01.4328 | 06.01.4367 | 06.01.1573 | Hygromycin + puromycin | 1.12E+04 |
| 06.01.4329 | 06.01.4330 | 06.01.4331 | 06.01.4332 | 06.01.4368 | 06.01.1582 | Hygromycin + puromycin | 1.24E+04 |
| 06.01.4333 | 06.01.4334 | 06.01.4335 | 06.01.4336 | 06.01.4369 | 06.01.1596 | Hygromycin + puromycin | 9.46E+03 |
| 06.01.4337 | 06.01.4338 | 06.01.4339 | 06.01.4340 | 06.01.4370 | 06.01.1614 | Hygromycin + puromycin | 2.31E+04 |
| 06.01.4341 | 06.01.4342 | 06.01.4343 | 06.01.4344 | 06.01.4371 | 06.01.1679 | Hygromycin + puromycin | 2.20E+03 |
| 06.01.4345 | 06.01.4346 | 06.01.4347 | 06.01.4348 | 06.01.4372 | 06.01.1687 | Hygromycin + puromycin | 6.61E+04 |
| 06.01.4349 | 06.01.4350 | 06.01.4351 | 06.01.4352 | 06.01.4373 | 06.01.1770 | Hygromycin + puromycin | 4.13E+04 |
| 06.01.4353 | 06.01.4354 | 06.01.4355 | 06.01.4356 | 06.01.4374 | 06.01.1757 | Hygromycin + puromycin | 4.25E+04 |
| 06.01.4357 | 06.01.4358 | 06.01.4359 | 06.01.4360 | 06.01.4375 | 06.01.1807 | Hygromycin + puromycin | 3.00E+04 |
| 06.01.4301 | 06.01.4302 | 06.01.4303 | 06.01.4304 | 06.01.4361 | n/a | Hygromycin + puromycin | 5.51E+02 |
| 06.01.4305 | 06.01.4306 | 06.01.4307 | 06.01.4308 | 06.01.4362 | n/a | Hygromycin + puromycin | 3.52E+02 |
| 06.01.4309 | 06.01.4310 | 06.01.4311 | 06.01.4312 | 06.01.4363 | n/a | Hygromycin + puromycin | 3.04E+02 |
| 06.01.4313 | 06.01.4314 | 06.01.4315 | 06.01.4316 | 06.01.4364 | n/a | Hygromycin + puromycin | 4.22E+02 |
| 06.01.4317 | 06.01.4318 | 06.01.4319 | 06.01.4320 | 06.01.4365 | n/a | Hygromycin + puromycin | 2.29E+02 |
| 06.01.4321 | 06.01.4322 | 06.01.4323 | 06.01.4324 | 06.01.4366 | n/a | Hygromycin + puromycin | 4.73E+02 |
| 06.01.4325 | 06.01.4326 | 06.01.4327 | 06.01.4328 | 06.01.4367 | n/a | Hygromycin + puromycin | 1.66E+03 |
| 06.01.4329 | 06.01.4330 | 06.01.4331 | 06.01.4332 | 06.01.4368 | n/a | Hygromycin + puromycin | 1.01E+03 |
| 06.01.4333 | 06.01.4334 | 06.01.4335 | 06.01.4336 | 06.01.4369 | n/a | Hygromycin + puromycin | 1.77E+03 |
| 06.01.4337 | 06.01.4338 | 06.01.4339 | 06.01.4340 | 06.01.4370 | n/a | Hygromycin + puromycin | 2.78E+02 |
| 06.01.4341 | 06.01.4342 | 06.01.4343 | 06.01.4344 | 06.01.4371 | n/a | Hygromycin + puromycin | 4.46E+02 |
| 06.01.4345 | 06.01.4346 | 06.01.4347 | 06.01.4348 | 06.01.4372 | n/a | Hygromycin + puromycin | 9.68E+02 |
| 06.01.4349 | 06.01.4350 | 06.01.4351 | 06.01.4352 | 06.01.4373 | n/a | Hygromycin + puromycin | 4.94E+02 |
| 06.01.4353 | 06.01.4354 | 06.01.4355 | 06.01.4356 | 06.01.4374 | n/a | Hygromycin + puromycin | 1.41E+02 |
| 06.01.4357 | 06.01.4358 | 06.01.4359 | 06.01.4360 | 06.01.4375 | n/a | Hygromycin + puromycin | 2.94E+02 |

The examples described above of the present disclosure are merely examples used for clearly describing the present application, instead of limiting the implementation modes of the present application. For those of ordinary skill in the art, other various forms of changes or variations could have also been made on the basis of the above-mentioned illustration. There is no need to exhaustively list all implementations herein, although it cannot be achieved. Any modifications, equivalent replacements, and improvements made without departing from the spirit and principle of the present application shall fall within the protection scope of the claims of the present application.

1. A method for integrating one or more exogenous nucleotide sequences into a mammalian host cell genome, wherein the method comprises integrating the one or more exogenous nucleotide sequences into the mammalian host cell genome using at least two transposon systems.

2. The method according to claim 1, wherein the at least two transposon systems are selected from: a Tol1 transposon system, a Tol2 transposon system, a Frog Prince transposon system, a Minos transposon system, a Hsmar1 transposon system, a Helraiser transposon system, a ZB transposon system, an Intruder transposon system, a SPINON transposon system, a TcBuster transposon system, a Passport transposon system, a Yabusame-1 transposon system, a Uribo2 transposon system, a PiggyBac (PB) transposon system, a Sleeping Beauty (SB) transposon system, and various variants or derivatives of the above-mentioned transposon systems.

3. The method according to claim 1, wherein the one or more exogenous nucleotide sequences are integrated into the mammalian host cell genome simultaneously or successively using the at least two transposon systems.

4. (canceled)

5. A mammalian cell, wherein the mammalian cell comprises at least two transposons integrated in the genome obtained using the method according to claim 1.

6. The mammalian cell according to claim 5, wherein the sequences of the at least two transposons do not overlap with each other.

7. The mammalian cell according to claim 5, wherein the at least two transposons are selected from: a Tol1 transposon, a Tol2 transposon, a Frog Prince transposon, a Minos transposon, a Hsmar1 transposon, a Helraiser transposon, a ZB transposon, an Intruder transposon, a SPINON transposon, a TcBuster transposon, a Passport transposon, a Yabusame-1 transposon, a Uribo2 transposon, a PiggyBac (PB) transposon, a Sleeping Beauty (SB) transposon, and various variants or derivatives of the above-mentioned transposons.

8. A method for constructing a lentivirus producing cell line, wherein the method comprises integrating the sequences of gag, pol and rev genes of a lentivirus, a sequence encoding a viral envelope protein, and a viral genome transcription cassette sequence carrying a nucleic acid fragment of interest into a host cell genome using at least two transposon systems.

9. (canceled)

10. The method according to claim 2, wherein the at least two transposon systems are selected from: a Tol1 transposon system, a Tol2 transposon system, a ZB transposon system, an Intruder transposon system, a TcBuster transposon system, a Yabusame-1 transposon system, a Uribo2 transposon system, a PB transposon system and a SB transposon system, and various variants or derivatives of the above-mentioned transposon systems.

11. The mammalian cell according to claim 5, wherein the mammalian cell is a lentivirus producing cell line.

12. The mammalian cell according to claim 7, wherein the at least two transposons are selected from: a Tol1 transposon, a Tol2 transposon, a ZB transposon, an Intruder transposon, a TcBuster transposon, a Yabusame-1 transposon, a Uribo2 transposon, a PB transposon and a SB transposon, and various variants or derivatives of the above-mentioned transposons.

13. The method according to claim 8, wherein the at least two transposon systems are selected from: a Tol1 transposon system, a Tol2 transposon system, a Frog Prince transposon system, a Minos transposon system, a Hsmar1 transposon system, a Helraiser transposon system, a ZB transposon system, an Intruder transposon system, a SPINON transposon system, a TcBuster transposon system, a Passport transposon system, a Yabusame-1 transposon system, a Uribo2 transposon system, a PiggyBac (PB) transposon system, a Sleeping Beauty (SB) transposon system, and various variants or derivatives of the above-mentioned transposon systems.

14. The method according to claim 13, wherein the at least two transposon systems are selected from: a Tol1 transposon system, a Tol2 transposon system, a ZB transposon system, an Intruder transposon system, a TcBuster transposon system, a Yabusame-1 transposon system, a Uribo2 transposon system, a PB transposon system and a SB transposon system, and various variants or derivatives of the above-mentioned transposon systems.
